SEQUENCE LISTING (1 sequence)

SEQ ID NO: 1 - DNA, 18 bp, Artificial sequence
  Synthetic oligonucleotide
ttagggttagggttaggg

(19) United States
(12) Patent Application Publication
(10) Pub. No.: US 2019/0298751 A1
(43) Pub. Date: Oct. 3, 2019

(54) 6-THIO-2'-DEOXYGUANOSINE (6-THIO-DG) RESULTS IN TELOMERASE DEPENDENT TELOMERE DYSFUNCTION AND CELL DEATH IN VARIOUS MODELS OF THERAPY-RESISTANT CANCER CELLS

(71) Applicants: The Board of Regents of the University of Texas System, Austin, TX (US); The Wistar Institute of Anatomy and Biology, Philadelphia, PA (US)

(72) Inventors: Jerry W. SHAY, Dallas, TX (US); Gao ZHANG, Philadelphia, PA (US)

(73) Assignees: The Board of Regents of the University of Texas System, Austin, TX (US); The Wistar Institute of Anatomy and Biology, Philadelphia, PA (US)

(21) Appl. No.: 16/304,538

(22) PCT Filed: May 26, 2017
(86) PCT No.: PCT/US2017/034706

§ 371 Date: Nov. 26, 2018

Related U.S. Application Data
(60) Provisional application No. 62/342,593, filed on May 27, 2016.

(51) Int. Cl. A61K 31/7076; A61P 35/00

(52) U.S. Cl. CPC A61K 31/7076; A61P 35/00

(57) ABSTRACT

The present disclosure provide for methods of using 6-thio-2′-deoxyguanosine (6-thio-dG) to treat telomerase-positive cancers that exhibit (a) one or more TERT promoter mutations, and/or (b) enriched telomere transcriptional signature(s). In particular, melanomas, including those who are not sensitive or have become resistant to immune checkpoint inhibition and/or MAPKi therapy are targets for this therapy.

### PRIORITY CLAIM

This application claims benefit of priority to U.S. Provisional Application Ser. No. 62/342,593, filed May 27, 2016, the entire contents of which are hereby incorporated by reference.

### BACKGROUND

### I. Field

The present disclosure relates to the fields of medicine, pharmacology and oncology. More particular, the disclosure relates to methods and compositions for treating cancers that express telomerase, a cellular reverse transcriptase that is express in 90% of all human cancers. In some embodiments, the cancer is melanoma.

### II. Related Art

Telomerase promoter mutations are highly prevalent in human tumors including melanoma. Telomere transcriptional signatures are enriched in a subset of therapy-naïve melanomas associated with worse overall survival, in BRAF-mutant intrinsically resistant melanoma cells that evade MAPK inhibitors (MAPKi), as well as in a subset of post-treatment tumor biopsies derived from patients who have disease progression on MAPKi or the immune checkpoint inhibitors. Herein, the efficacy of a telomerase-directed nucleoside, 6-thio-2′-deoxyguanosine (6-thio-dG) that results in telomere dysfunction and cell death in various models of therapy-resistant cells, is demonstrated. Furthermore, 6-thio-dG significantly inhibits tumor growth of primary tumor biopsy cultures derived from patients who had disease progression on multiple therapies including anti-CTLA-4 or anti-PD1.

Immune checkpoint blockade inhibitors and mitogen-activated protein kinase (MAPK) inhibitors have emerged as first-line therapies for patients with advanced melanomas. Despite highly encouraging successes, many patients do not respond and even those that do initially respond, many patients ultimately relapse and are left with limited options. As a result, there is an unmet and urgent need to prolong disease control for patients who fail multiple therapies.

### SUMMARY

Thus, in accordance with the present disclosure, there is provided a method of treating a subject with cancer comprising administering to said subject a therapeutically effective amount of 6-thio-2′-deoxyguanosine (6-thio-dG), wherein cells of said cancer are telomerase-positive and exhibit (a) one or more TERT promoter mutations, and/or (b) enriched telomere transcriptional signature(s). Also provided is a method of treating a subject with melanoma comprising administering to said subject a therapeutically effective amount of 6-thio-2′-deoxyguanosine (6-thio-dG), wherein melanoma is resistant to an immunotherapy and/or MAPKi therapy.

The subject may have had disease progression during or after platinum-based therapy, radiotherapy, immunotherapy and/or MAPKi therapy. The immunotherapy may be an immune checkpoint inhibitor, such as anti-CTLA4 therapy or anti-PD1 therapy. The MAPKi therapy may be an anti-MEK therapy, an anti-Raf therapy, and anti-p38 MAPK therapy, and anti-JNK therapy, and anti-ERK therapy, or an anti MNK therapy, such as vemurafenib, sorafenib, dabrafenib, trametinib, selumetinib, losapimod, GSK2118436, PD0325901, PLX4032 or PLX4720. The cancer may be a B-Raf-mutated cancer, such as a Braf^{V600} mutant. The cancer may be a lung cancer, a melanoma, pancreatic cancer or an ovarian cancer. The cancer may be recurrent, metastatic and/or multi-drug resistant.

The enriched telomere transcription signature may be a telomere maintenance signature or a packaging of telomere ends signature. The therapeutically effective amount of 6-thio-dG may be between about 0.5 mg/kg and 5.0 mg/kg. The 6-thio-dG is administered more than once, such as twice daily, daily, every other day, twice a week, weekly, every other week, every three weeks, or monthly. The 6-thio-dG is administered systemically, such as orally or intravenously, or administered intratumorally, or local or regional to a tumor site. The subject may be a human subject or a non-human mammalian subject.

The method may further comprise treating said subject with a second cancer therapy, such as an immunotherapy, such as ipilumumab and nivolumab or combination of ipilumumab and nivolumab, a radiotherapy, a neoadjuvant chemotherapy (such as plantinum/taxane), a toxin therapy, a hormonal therapy or surgery. The second cancer therapy may be administered at the same time or after 6-thio-dG, or may be administered before 6-thio-dG.

The treatment may result in one or more of impaired cancer cell viability, cancer cell apoptosis, cancer cell senescence in surviving cancer cells, and progressively shortened telomeres in surviving cancer cells. The treatment may result in one or more of increased subject survival, reduced tumor burden, reduction in primary tumor size, reduced metastasis, induction of remission, reduced subject hospitalization and increased subject comfort.

The method may further comprise assessing a cancer cell from said subject from one or more of (a) TERT promoter mutations, (b) enriched telomere transcriptional signature(s), (c) increased AXL expression, (d) increased PDGFRβ expression, and/or (e) one or more B-Raf mutations. The enriched telomere transcription signature may be a telomere maintenance signature or a packaging of telomere ends signature.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the disclosure will be better appreciated and understood when considered in conjunction with the following description and the accompanying drawings. It should be understood, however, that the following description, while indicating various embodiments of the disclosure and numerous specific details thereof, is given by way of illustration and not of limitation. Many substitutions, modifications, additions and/or rearrangements may be made within the scope of the disclosure without departing from the spirit thereof, and the disclosure includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B show long-term cell growth assay of 12 non-melanoma cancer cell lines (FIG. 1A) and 12 BRAF-mutant melanoma cell lines (FIG. 1B) that were treated with 6-thio-dG (6dG) or BIBR 1532 (BIBR) at indicated doses for 9 to 12 days. Cells were then fixed and stained with crystal violet. Representative image of 2 biological replicates were shown for each experimental condition.

FIG. 2 shows mutational frequencies of TERT, BRAF and NRAS in Wistar melanoma cell lines, Wistar PDX, Wistar PDX that are resistant to MAPKi and TCGA SKCM data sets.

FIGS. 3A-3E show long-term cell growth assay of 16 BRAF-mutant melanoma cell lines that were treated with 6-thio-dG or PLX4720 at indicated doses for 12 days (FIG. 3A). Cells were then fixed and stained with crystal violet. Representative image of 2 biological replicates were shown for each experimental condition; percentage of apoptotic and dead cells indicated as PSVue 643+ cells in 12 BRAF-mutant melanoma cell lines treated with PLX4720 or 6-thio-dG (FIG. 3B) or in the A375 cell line treated with 6-thio-dG (FIG. 3C) at indicated doses for 120 hours. Cells were then harvested and co-stained with PSVue643 and Propidium iodide (PI). The average of 2 biological replicates are representative of 2 independent experiments; SA-β-gal staining of 4 BRAF-mutant melanoma cell lines treated with 6-thio-dG at 5 μM for 9 days (FIG. 3D). A representative image of 3 biological replicates was shown for each experimental sample; and tumor volumes of 1205Lu xenografts that were treated for 12 days with the vehicle control or 6-thio-dG at indicated doses (FIG. 3E).

FIG. 4 shows percentage of apoptotic and dead cells in normal skin melanocytes (upper panel) or keratinocytes (lower panel) that were treated with the vehicle control or 6-thio-dG at indicates doses for 120 hours. Cells were then harvested and co-stained with PSVue643 and Propidium iodide (PI). Representative samples of 2 independent experiments.

FIGS. 5A-5G show the heatmap of RNA-seq data depicting genes that were most significantly down-regulated in A375 cells treated with 6-thio-dG (6dG) or BIBR 1532 (BIBR) for 72 hours (FIG. 5A). 2 biological replicates were shown for each experimental condition; the plot of Biocarta gene sets that were most significantly altered in A375 cells treated with 6-thio-dG (6dG) (FIG. 5B) or BIBR 1532 (BIBR) (FIG. 5C) as determined by the hypergeometric differential analysis; the ssGSEA plot of “Telomere” and “Cell Cycle” gene sets that were significantly altered in A375 cells treated with BIBR 1532 (BIBR) or 6-thio-dG (6dG) (FIG. 5D); the heatmap of RPPA data depicting 30 proteins that were most significantly down-regulated in A375 cells treated with 6-thio-dG (6dG) for 72 hours (FIG. 5E). 2 biological replicates were shown for each experimental condition; and Western blotting of proteins that were down-regulated in A375 cells treated with 6-thio-dG (6dG) for 48 and 72 hours (FIG. 5F) or 6-thio-dG (6dG), GRN163L (GRN) or BIBR 1532 (BIBR) for 48 hours (FIG. 5G) as identified by RPPA.

FIGS. 6A-6G show the heatmaps of RPPA data of ARID1A, AXL, PDGFRβ and PLK for A375, UACC-903 and WM9 cells that were treated with the control or 6-thio-dG for 120 hours (FIG. 6A); Immunoblot analysis of proteins in samples shown in FIG. 6A (FIG. 6B); heatmaps of RPPA data of ARID1A, AXL, PDGFRβ and PLK1 for A375 cells that were treated with the control or MAPKi at indicated doses for 120 hours (FIG. 6C); Immunoblot analysis of proteins in samples shown in (FIGS. 6C-6D) (FIG. 6E); the heatmaps of RPPA data of ARIDIA, AXL, PDGFRβ and PLK for A375, UACC-903 and WM9 parental cells and cells that acquired resistance to the BRAF inhibitor PLX4720 (BR) or cells that acquired resistance to the combination of PLX4720 and the MEK inhibitor PD0325901 (CR) (FIG. 6F); and long-term cell growth assay of 3 BRAF-mutant melanoma cell lines treated with 6-thio-dG at 5 μM and the BRAF inhibitor GSK2118436 at 1 μM either as single agent or in combination for 15 days. Ctrl: the vehicle control (FIG. 6G).

FIGS. 7A-7L show percentage of apoptotic and dead cells indicated as PSVue 643+ cells for LOX-IMVI BR cells that were treated with the control or 6-thio-dG at indicated doses for 120 hours (FIG. 7A). The average of 2 biological replicates was plotted, and data are representative of 2 independent experiments; the telomere length was measured in samples treated with 6-thio-dG at 0 and 5 μM as shown in FIG. 7A (FIG. 7B); co-staining of γ-H2AX antibody (double strand DNA damage marker) with an in situ telomere specific hybridization probe (FITC-conjugated telomere sequence (TIAGGG)3 peptide nucleic acid) in samples shown in FIG. 7B (FIG. 7C); quantification of DNA damage foci (FIG. 7D) and TIF (FIG. 7E) for samples shown in FIG. 7C; the ssGSEA plot of “Telomere” and “Cell Cycle” gene sets that were significantly altered in LOX-IMVI BR cells treated with BIBR 1532 (BIBR) or 6-thio-dG (6dG) (FIG. 7F); the heatmap of RPPA data depicting 30 proteins that were most significantly down-regulated in LOX-IMVI BR cells treated with 6-thio-dG (6dG) for 72 hours (FIG. 7G) (2 biological replicates were shown for each experimental condition); Immunoblot analysis of AXL and PLK1 for LOX-IMVI BR cells treated with the control or 6-thio-dG for 120 hours (FIG. 7H); long-term cell growth assay of 7 melanoma cell lines that acquired resistance to MAPKi treated with 6-thio-dG at indicated doses for 12 days (FIG. 7I) (Cells were then fixed and stained with crystal violet. A representative image of 2 biological replicates is shown for each experimental condition); and tumor volumes of xenografts of WM9 BR (FIG. 7J), LOX-IMVI BR (FIG. 7K) and UACC-903 BR (FIG. 7L) cells that were treated with the vehicle control or 6-thio-dG.

FIGS. 8A-8D show long-term cell growth assay of 4 melanoma cell lines that acquired resistance to MAPKi treated with 6-thio-dG at indicated doses for 12 days (FIG. 8A) (Cells were then fixed and stained with crystal violet. A representative image of 2 biological replicates is shown for each experimental condition); the heatmaps of RPPA data depicting 30 proteins that were most significantly down-regulated in eight BR cell lines (FIG. 8B), 1205Lu xenografts (FIG. 8C) and UACC-903 BR and LOX-IMVI BR xenografts (FIG. 8D) treated with 6-thio-dG.

FIGS. 9A-9F show the heatmap of enrichment scores of six gene sets in TCGA melanoma (FIG. 9A) and normal skin, nevi, and melanoma (FIG. 9B); Kaplan-Meier survival curves for TCGA melanoma cases which were divided into 2 subgroups with high and low enrichment scores of two telomere transcriptional gene sets (FIG. 9C); the heatmap of two telomere transcriptional gene signatures, two melanoma-specific gene sets and two MAPK pathway-related gene sets in GSE61992 in which transcriptomes of patients' paired pre- and post-treatment tumor biopsies were profiled (FIG. 9D); the GSEA plot of the “Biocarta Tel Pathway” gene set that was enriched in post-treatment tumor biopsies derived from patients Memorial Sloan Kettering Cancer Center who did not experience long-term benefit from ipilimumab (FIG. 9E); the GSEA plot of the “Telomere Maintenance” (upper panel) and “Packaging of Telomere Ends” (lower panel) gene sets that were enriched in the on-treatment tumor biopsy derived from a patient (ID #39) at Massachusetts General Hospital who did not experience long-term benefit from pembrolizumab (FIG. 9F).

FIGS. 10A-10D show the heatmaps of two telomere transcriptional gene signatures (FIG. 10A), two melanoma-specific gene sets (FIG. 10B), and two MAPK pathway-related gene sets (FIG. 10C) in three datasets in which transcriptomes of paired pre- and post-treatment tumor biopsies derived from patients who progressed on MAPKi were profiled. Expression of TERT transcript was determined by quantitative PCR experiment in paired pre-, on- and post-treatment tumor biopsies derived from patients who were treated with MAPKi at Massachusetts General Hospital (FIG. 10D).

FIG. 11 shows the heatmaps of two telomere transcriptional gene signatures, two melanoma-specific gene sets and two MAPK pathway-related gene sets in three datasets in which transcriptomes of paired pre- and post-treatment tumor biopsies derived from 12 patients who were treated with immune checkpoint blockade therapies were profiled.

FIGS. 12A-12I show long-term cell growth assay of 6 patient- or mouse-derived cultures that were treated with 6-thio-dG at indicated doses for 12 days (FIG. 12A) (Cells were then fixed and stained with crystal violet. A representative image of 2 biological replicates is shown for each experimental sample); tumor volumes of xenografts of 5 patient- or mouse-derived cultures treated with the vehicle control or 6-thio-dG (FIGS. 12B-12F); the heatmap of RPPA data depicting 30 proteins that were most significantly down-regulated in xenografts of 13-456-5-3 (FIG. 12G), 13-456-3-3 (FIG. 12H) and WM4265-2 (FIG. 12I) treated with 6-thio-dG (6dG).

FIG. 13 shows the heatmaps of RPPA data depicting 30 proteins that were most significantly down-regulated in 13-456-3-3, 13-456-5-3, WM4265-1 and WM4265-2 cells treated with 6-thio-dG.

FIGS. 14A-14M show long-term cell growth assay of G43 cells treated with 6-thio-dG at indicated doses for 12 days (FIG. 14A) (Cells were then fixed and stained with crystal violet. A representative image of 2 biological replicates is shown for each experimental condition); tumor volume of xenografts of G43 cells treated with the vehicle control or 6-thio-dG (FIG. 14B); the heatmap of RPPA data depicting 30 proteins that were most significantly down-regulated in G43 cells treated with 6-thio-dG in vitro (FIG. 14C) or G43 tumors treated with 6-thio-dG in vivo (FIG. 14D); the relative cell viability of PEO1 and PEO1-derived cell lines treated with carboplatin (Carbo) or 6-thio-dG at indicated doses for 5 days (FIGS. 14E-14J); Immunofluorescence staining of the patient-derived culture WO-24-2 with anti-PAX8 (red) and cytokeratin 7 (green) (FIG. 14K); and the relative cell viability of the patient-derived culture WO-24-2 treated with carboplatin (Carbo) (FIG. L) or 6-thio-dG (FIG. M) at indicated doses for 5 days.

### DETAILED DESCRIPTION OF THE PREFERRED EMBODIMENTS

In telomerase-positive melanoma cells with a high frequency of TERT promoter mutations, the present inventors herein demonstrate that 6-thio-dG exhibits strong anti-tumor effects by concomitantly inducing telomere dysfunction and inhibiting ARIDIA, AXL, PDGFRβ and PLK1. 6-thio-dG not only abrogates intrinsic and acquired drug resistance to MAPK inhibitors, but also inhibits the growth of tumors that have become resistant to immune checkpoint blockade therapies. Furthermore, the efficacy of 6-thio-dG in therapy-resistant mouse pancreatic cancer and human ovarian cancer cells is demonstrated. Thus, 6-thio-dG presents a viable approach to prolonged disease control of therapy-resistant tumors as an effective salvage therapy. These and other aspects of the disclosure are set out in detail below.

### I. TELOMERASE AND TELOMERE DYSFUNCTION

During mitosis, cells make copies of their genetic material. Half of the genetic material goes to each new daughter cell. To make sure that information is successfully passed from one generation to the next, each chromosome has a special protective cap called a telomere located at the end of its “arms.” Telomeres are controlled by the presence of the enzyme telomerase.

A telomere is a repeating DNA sequence (for example, TTAGGG) at the end of the body's chromosomes. The telomere can reach a length of 15,000 base pairs. Telomeres function by preventing chromosomes from losing base pair sequences at their ends. They also stop chromosomes from fusing to each other. However, each time a cell divides, some of the telomere is lost (usually 25-200 base pairs per division). When the telomere becomes too short, the chromosome reaches a “critical length” and can no longer replicate. This means that a cell becomes old and dies by a process called apoptosis. Telomere activity is controlled by two mechanisms: erosion and addition. Erosion, as mentioned, occurs each time a cell divides. Addition is determined by the activity of telomerase.

Telomerase, also called telomere terminal transferase, is an enzyme made of protein and RNA subunits that elongates chromosomes by adding TrAGGG sequences to the end of existing chromosomes. Telomerase is found in fetal tissues, adult germ cells, and also tumor cells. Telomerase activity is regulated during development and has a very low, almost undetectable activity in somatic (body) cells. Because these somatic cells do not regularly use telomerase, they age. The result of aging cells is an aging body. If telomerase is activated in a cell, the cell will continue to grow and divide. This “immortal cell” theory is important in two areas of research: aging and cancer.

Cellular aging, or senescence, is the process by which a cell becomes old and dies. It is due to the shortening of chromosomal telomeres to the point that the chromosome reaches a critical length. Cellular aging is analogous to a wind up clock. If the clock stays wound, a cell becomes immortal and constantly produces new cells. If the clock winds down, the cell stops producing new cells and dies. Cells are constantly aging. Being able to make the body's cells live forever certainly creates some exciting possibilities. Telomerase research could therefore yield important discoveries related to the aging process.

Cancer cells are a type of malignant cell. The malignant cells multiply until they form a tumor that grows uncontrollably. Telomerase has been detected in human cancer cells and is found to be 10-20 times more active than in normal body cells. This provides a selective growth advantage to many types of tumors. If telomerase activity was to be turned off, then telomeres in cancer cells would shorten, just like they do in normal body cells. This would prevent the cancer cells from dividing uncontrollably in their early stages of development. In the event that a tumor has already thoroughly developed, it may be removed and anti-telomerase therapy could be administered to prevent relapse. In essence, preventing telomerase from performing its function would change cancer cells from immortal to mortal.

### II. MAPK INHIBITION

A mitogen-activated protein kinase (MAPK or MAP kinase) is a type of protein kinase that is specific to the amino acids serine, threonine, and tyrosine (i.e., a serine/threonine-specific protein kinase). MAPKs are involved in directing cellular responses to a diverse array of stimuli, such as mitogens, osmotic stress, heat shock and pro-inflammatory cytokines. They regulate cell functions including proliferation, gene expression, differentiation, mitosis, cell survival, and apoptosis.

MAP kinases are found in eukaryotes only, but they are fairly diverse and encountered in all animals, fungi and plants, and even in an array of unicellular eukaryotes. MAPKs belong to the CMGC (CDK/MAPK/GSK3/CLK) kinase group. The closest relatives of MAPKs are the cyclin-dependent kinases (CDKs).

Since the ERK signaling pathway is involved in both physiological and pathological cell proliferation, it is natural that ERK1/2 inhibitors would represent a desirable class of antineoplastic agents. Indeed, many of the proto-oncogenic “driver” mutations are tied to ERK1/2 signaling, such as constitutively active (mutant) receptor tyrosine kinases, Ras or Raf proteins. Although no MKK1/2 or ERK1/2 inhibitors were developed for clinical use, kinase inhibitors that also inhibit Raf kinases (e.g., Sorafenib) are successful antineoplastic agents against various types of cancer.

JNK kinases are implicated in the development of insulin resistance in obese individuals as well as neurotransmitter excitotoxicity after ischemic conditions. Inhibition of JNK1 ameliorates insulin resistance in certain animal models. Mice that were genetically engineered to lack a functional JNK3 gene—the major isoform in brain—display enhanced ischemic tolerance and stroke recovery. Although small-molecule JNK inhibitors are under development, none of them proved to be effective in human tests yet. Interestingly, a peptide-based JNK inhibitor (AM-111, a retro-inverse D-motif peptide from JIP1, formerly known as XG-102) is also under clinical development for sensorineural hearing loss.

p38 was once believed to be a perfect target for anti-inflammatory drugs. Yet the failure of more than a dozen chemically different compounds in the clinical phase suggests that p38 kinases might be poor therapeutic targets in autoimmune diseases. Many of these compounds were found to be hepatotoxic to various degree and tolerance to the anti-inflammatory effect developed within weeks.

### III. IMMUNE CHECKPOINT INHIBITION

Immune checkpoints are molecules in the immune system that either turn up a signal (co-stimulatory molecules) or turn down a signal. Many cancers protect themselves from the immune system by inhibiting the T cell signal. Since around 2010 inhibitory checkpoint molecules have been increasingly considered as new targets for cancer immunotherapies due to the effectiveness of two checkpoint inhibitor drugs that were initially indicated for advanced melanoma—Yervoy®, from Bristol-Myers Squibb, and Keytruda®, from Merck.

Four stimulatory checkpoint molecules are members of the tumor necrosis factor (TNF) receptor superfamily +CD27, CD40, OX40, GITR and CD137. Another two stimulatory checkpoint molecules belongs to the B7-CD28 superfamily—CD28 itself and ICOS.

CD27. This molecule supports antigen-specific expansion of naïve T cells and is vital for the generation of T cell memory. CD27 is also a memory marker of B cells. CD27's activity is governed by the transient availability of its ligand, CD70, on lymphocytes and dendritic cells. CD27 costimulation is known to suppress Th17 effector cell function. The American biotech company Celldex Therapeutics is working on CDX-1127, an agonistic anti-CD27 monoclonal antibody which in animal models has been shown to be effective in the context of T cell receptor stimulation.

CD28. This molecule is constitutively expressed on almost all human CD4+ T cells and on around half of all CD8 T cells. Binding with its two ligands are CD80 and CD86, expressed on dendritic cells, prompts T cell expansion. CD28 was the target of the TGN1412 ‘superagonist’ which caused severe inflammatory reactions in the first-in-man study in London in March 2006.

CD40. This molecule, found on a variety of immune system cells including antigen presenting cells has CD40L, otherwise known as CD154 and transiently expressed on the surface of activated CD4+ T cells, as its ligand. CD40 signaling is known to ‘license’ dendritic cells to mature and thereby trigger T-cell activation and differentiation. A now-defunct Seattle-based biotechnology company called VLST in-licensed an anti-CD40 agonist monoclonal antibody from Pfizer in 2012. The Swiss pharmaceutical company Roche acquired this project when VLST was shut down in 2013.

CD122. This molecule, which is the Interleukin-2 receptor beta sub-unit, is known to increase proliferation of CD8+ effector T cells. The American biotechnology company Nektar Therapeutics is working on NKTR-214, a CD122-biased immune-stimulatory cytokine Phase I results announced in November 2016.

CD137. When this molecule, also called 4-1BB, is bound by CD137 ligand, the result is T-cell proliferation. CD137-mediated signaling is also known to protect T cells, and in particular, CD8+ T cells from activation-induced cell death. The German biotech company Pieris Pharmaceuticals has developed an engineered lipocalin that is bi-specific for CD137 and HER2.

OX40. This molecule, also called CD134, has OX40L, or CD252, as its ligand. Like CD27, OX40 promotes the expansion of effector and memory T cells, however it is also noted for its ability to suppress the differentiation and activity of T-regulatory cells, and also for its regulation of cytokine production. OX40's value as a drug target primarily lies it the fact that, being transiently expressed after T-cell receptor engagement, it is only upregulated on the most recently antigen-activated T cells within inflammatory lesions. Anti-OX40 monoclonal antibodies have been shown to have clinical utility in advanced cancer.^{j} The pharma company AstraZeneca has three drugs in development targeting OX40: MEDI0562 is a humanized OX40 agonist; MEDI6469, murine OX4 agonist; and MEDI6383, an OX40 agonist.

GITR (Glucocorticoid-Induced TNFR Family Related Gene). GITR prompts T cell expansion, including Treg expansion. The ligand for GITR is mainly expressed on antigen presenting cells. Antibodies to GITR have been shown to promote an anti-tumor response through loss of Treg lineage stability. The biotech company TG Therapeutics is working on anti-GITR antibodies.

ICOS. This molecule, short for Inducible T-cell costimulator, and also called CD278, is expressed on activated T cells. Its ligand is ICOSL, expressed mainly on B cells and dendritic cells. The molecule seems to be important in T cell effector function. The American biotechnology company Jounce Therapeutics is developing an ICOS agonist.

A2AR. The Adenosine A2A receptor is regarded as an important checkpoint in cancer therapy because adenosine in the immune microenvironment, leading to the activation of the A2a receptor, is negative immune feedback loop and the tumor microenvironment has relatively high concentrations of adenosine.

B7-H3 (CD276). B7-H3 was originally understood to be a co-stimulatory molecule but is now regarded as co-inhibitory. The American biotechnology company MacroGenics is working on MGA271 is an Fc-optimized monoclonal antibody that targets B7-H3. B7-H3's receptors have not yet been identified.

B7-H4 (VTCN1). This molecule is expressed by tumor cells and tumor-associated macrophages and plays a role in tumor escape.

BTLA. This molecule, short for B and T Lymphocyte Attenuator and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA.

CTLA-4 (Cytotoxic T-Lymphocyte-Associated protein 4; CD152). This molecule is the target of Bristol-Myers Squibb's melanoma drug Yervoy®, which gained FDA approval in March 2011. Expression of CTLA-4 on Treg cells serves to control T cell proliferation.

IDO (Indoleamine 2,3-dioxygenase). This is a tryptophan catabolic enzyme with immune-inhibitory properties. Another important molecule is TDO, tryptophan 2,3-dioxygenase. IDO is known to suppress T and NK cells, generate and activate Tregs and myeloid-derived suppressor cells, and promote tumour angiogenesis. The American biotechnology companies Newlink Genetics and Incyte are working on IDO pathway inhibitors.

KIR (Killer-cell Immunoglobulin-like Receptor). This is a receptor for MHC Class I molecules on Natural Killer cells. Bristol-Myers Squibb is working on Lirilumab, a monoclonal antibody to KIR.

LAG3 (Lymphocyte Activation Gene-3) works to suppress an immune response by action to Tregs as well as direct effects on CD8+ T cells. Bristol-Myers Squibb is in Phase I with an anti-LAG3 monoclonal antibody called BMS-986016.

PD-1 (Programmed Death 1 (PD-1) receptor). PD-1 has two ligands, PD-L and PD-L2. This checkpoint is the target of Keytruda®, which gained FDA approval in September 2014. An advantage of targeting PD-1 is that it can restore immune function in the tumor microenvironment.

TIM-3 (T-cell Immunoglobulin domain and Mucin domain 3). TIM-3 is expressed on activated human CD4+ T cells and regulates Th1 and Th17 cytokines. TIM-3 acts as a negative regulator of Th1/Tc1 function by triggering cell death upon interaction with its ligand, galectin-9.

VISTA. Short for V-domain Ig suppressor of T cell activation, VISTA is primarily expressed on hematopoietic cells so that consistent expression of VISTA on leukocytes within tumors may allow VISTA blockade to be effective across a broad range of solid tumors.

### IV. THERAPIES

A. Target Cancers

In accordance with the present disclosure, 6-thio-dG can be employed to treat a variety of cancer types. In general, melanomas, lung cancers, pancreatic cancers and ovarian cancers. However, more generally, tumors expressing telomerase, including those having TERT promoter mutations and enriched telomere transcription signatures (e.g., a telomere maintenance signature and/or a packaging of telomere ends signature). Moreover, a variety of therapy-resistant cancers are responsive to 6-thio-dG therapy.

By way of background, the inventors examined publically available databases and found that patients with telomerase mutations and telomere transcriptional signature had overall worse survival in a variety of types of advanced melanoma (such as those with BRAF mutations). The inventors hypothesized that patients having failed BRAF inhibitor therapy, such as MAPK inhibitors (downstream of BRAF activating mutations), would be good candidates for 6-thio-dG therapy. This would be important given that patients that fail MAPKi have few therapeutic options remaining. The inventors examined cells from patients that failed MAPKi and found they were very sensitive to 6-thio-dG. Moreover, over 50% of advanced melanoma patients do not respond to immunotherapy, including powerful MAPKi and immune checkpoint therapies, leaving a major gap in therapeutic options that can be filed by 6-thio-dG.

Melanoma is in particular of interest here. It is known that TERT promoter mutations are very common in melanoma, as are enriched telomere transcriptional signatures. For example, the inventors have performed targeted sequencing that revealed a high frequency of TERT promoter mutations that are identified in 73.2% of 97 Wistar's melanoma cell lines, 67.4% of 172 Wistar's melanoma treatment-naïve PDXs, and 82.9% of 35 Wistar's MAPK inhibitors-resistant PDXs (data not shown), which is consistent with the finding that TERT promoter mutations occur in 64.3% of 115 TCGA melanoma patients. Notably, the frequency of TERT promoter mutations is significantly higher in Wistar's MAPK inhibitors-resistant PDXs than that in the TCGA melanoma patient cohort (Fisher's exact test; p=0.04). Thus, melanoma is considered a prime target for 6-thio-dG therapy.

Skin cancer is the most common form of cancer in the U.S., and melanoma is the deadliest form of skin cancer. Over half of the people in the U.S. diagnosed with melanoma will be diagnosed with invasive (Stage I, II, III or IV). Melanoma is the leading cause of cancer death in young women ages 25-30, and the second leading cause of cancer death in women ages 30-35. In ages 15-29, melanoma is the second most commonly diagnosed cancer. The incidence of people under 30 developing melanoma is increasing faster than any other demographic group, soaring by 50% in women since 1980.

Commonly prescribed immune stimulants for the treatment of melanoma include biologic agents such as antibodies, interferons and interleukins, which are administered in much higher doses than are usually present in the body.

T-VEC (Imlygic®) received FDA approval in October 2015. Imlygic is a genetically modified oncolytic viral therapy indicated for the local treatment of unresectable cutaneous, subcutaneous and nodal lesions in patients whose melanoma has recurred after initial surgery. Imlygic is a genetically modified herpes simplex virus type I designed to replicate within tumors, causing tumors to rupture (cell death).

Ipi+Nivo (Ipilimumab®+Nivolumab®) combination received accelerated FDA approval in September 2015 based on improved response rates and progression-free survival in previously treated patients.

Nivolumab (Opdivo®) was approved in November 2015 as a first line therapy (previously untreated) for melanoma patients who do not have a positive BRAF V600 mutation. It was previously approved in 2014 for patients whose disease had progressed following ipilimumab and, if BRAF V600 mutation positive, also a BRAF inhibitor. It is the second anti-PD-1 drug to be approved for the treatment of unresectable (cannot be removed by surgery) or advanced (metastatic) melanoma, but the only anti-PD-1 therapy approved as a single agent for first-line use in patients with advanced BRAF V600 wild-type (not mutated) melanoma.

Pembrolizumab (Keytruda®) received accelerated approval in 2014 for demonstrating durable responses in patients whose disease has progressed following ipilimumab and, if BRAF V600 mutation positive, also a BRAF inhibitor. Randomized trials are in progress to assess the ability of pembrolizumab to improve time to progression and overall survival. Keytruda is the first anti-PD-drug to be approved by the FDA for melanoma.

Ipilimumab (Yervoy®), which stimulates T cells, was approved by the FDA in 2011. It was the first drug in 13 years to be approved for the treatment of metastatic melanoma. Randomized trials have shown an improvement in overall survival in patients with either previously treated or untreated advanced melanoma. In addition, in October 2015, Yervoy was approved as adjuvant therapy in patients with Stage III melanoma. Patients and physicians should be aware that immune-mediated toxicities may be severe so good communication with your physician will allow early identification and successful treatment. Common side effects include: tiredness, diarrhea, itching and rash.

Peginterferon alpha 2-b (Sylatron®) is the FDA-approved standard treatment for patients with metastatic melanoma that has been surgically resected and that are at high risk for recurrence (i.e., for adjuvant therapy). Analyses of randomized trials of interferon used in an adjuvant setting show that it can lengthen the time of melanoma recurrence, but it does not appear to prolong survival.

Interleukin-2 (IL-2; Proleukin®) was the first immunotherapy to be approved for metastatic melanoma (1998) and was approved on the basis of long-lasting complete response. Randomized trials of IL-2 have not been conducted, so precise information on long-term overall survival is not available.

Any of the preceding may be used prior to 6-thio-dG, even if the patient has progress. and/or may be used in combination with 6-thio-dG (see below).

B. Pharmaceutical Formulations and Routes of Administration

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render drugs stable and allow for uptake by target cells. Aqueous compositions of the present invention comprise an effective amount of the drug dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

The active compositions of the present invention may include classic pharmaceutical preparations. Administration of these compositions according to the present invention may be via any common route so long as the target tissue is available via that route, but generally including systemic administration. This includes oral, nasal, or buccal. Alternatively, administration may be by intradermal, subcutaneous, intramuscular, intraperitoneal or intravenous injection, intratumoral or by direct injection into muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

C. Combination Therapies

In the context of the present disclosure, it also is contemplated 6-thio-dG could be used in conjunction with chemo- or radiotherapeutic intervention, or other treatments. It also may prove effective, in particular, to combine 6-thio-dG with other therapies that target different aspects of cancer cell function.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with 6-thio-dG and at least one other agent. These compositions would be provided in a combined amount effective to kill or inhibit proliferation of the cell. This process may involve contacting the cells with 6-thio-dG and the other agent(s) or factor(s) at the same time. This may be achieved by contacting the cell with a single composition or pharmacological formulation that includes both agents, or by contacting the cell with two distinct compositions or formulations, at the same time, wherein one composition includes the interferon prodrugs according to the present disclosure and the other includes the other agent.

Alternatively, the 6-thio-dG therapy may precede or follow the other agent treatment by intervals ranging from minutes to weeks. In embodiments where the other agent and the interferon prodrugs are applied separately to the cell, one would generally ensure that a significant period of time did not expire between each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either interferon prodrugs or the other agent will be desired. Various combinations may be employed, where 6-thio-dG therapy is “A” and the other therapy is “B”, as exemplified below:

| | | | | | |
|---|---|---|---|---|---|
| A/B/A | B/A/B | B/B/A | A/A/B | B/A/A | A/B/B |
| B/B/B/A | B/B/A/B | A/A/B/B | A/B/A/B | A/B/B/A | B/B/A/A |
| B/A/B/A | B/A/A/B | B/B/B/A | A/A/A/B | B/A/A/A | A/B/A/A |
| A/A/B/A | A/B/B/B | B/A/B/B | B/B/A/B | | |

Other combinations are contemplated. Again, to achieve cell killing, both agents are delivered to a cell in a combined amount effective to kill the cell.

Agents or factors suitable for cancer therapy include any chemical compound or treatment method that induces DNA damage when applied to a cell. Such agents and factors include radiation and waves that induce DNA damage such as, irradiation, microwaves, electronic emissions, and the like. A variety of chemical compounds, also described as “chemotherapeutic” or “genotoxic agents,” may be used. This may be achieved by irradiating the localized tumor site; alternatively, the tumor cells may be contacted with the agent by administering to the subject a therapeutically effective amount of a pharmaceutical composition.

Various classes of chemotherapeutic agents are comtemplated for use with the present disclosure. For example, selective estrogen receptor antagonists (“SERMs”), such as Tamoxifen, 4-hydroxy Tamoxifen (Afimoxfene), Falsodex, Raloxifene, Bazedoxifene, Clomifene, Femarelle, Lasofoxifene, Ormeloxifene, and Toremifene.

Chemotherapeutic agents contemplated to be of use, include, e.g., camptothecin, actinomycin-D, mitomycin C. The disclosure also encompasses the use of a combination of one or more DNA damaging agents, whether radiation-based or actual compounds, such as the use of X-rays with cisplatin or the use of cisplatin with etoposide. The agent may be prepared and used as a combined therapeutic composition.

Heat shock protein 90 is a regulatory protein found in many eukaryotic cells. HSP90 inhibitors have been shown to be useful in the treatment of cancer. Such inhibitors include Geldanamycin, 17-(Allylamino)-17-demethoxygeldanamycin, PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for doxorubicin, to 35-50 mg/m² for etoposide intravenously or double the intravenous dose orally. Microtubule inhibitors, such as taxanes, also are contemplated. These molecules are diterpenes produced by the plants of the genus Taxus, and include paclitaxel and docetaxel.

Epidermal growth factor receptor inhibitors, such as Iressa, mTOR, the mammalian target of rapamycin (also known as FK506-binding protein 12-rapamycin associated protein 1 (FRAP1)), is a serine/threonine protein kinase that regulates cell growth, cell proliferation, cell motility, cell survival, protein synthesis, and transcription. Rapamycin and analogs thereof (“rapalogs”) are therefore contemplated for use in cancer therapy in accordance with the present disclosure. Another EGFR inhibitor of particular utility here is Gefitinib.

Another possible therapy is TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenesis and viral replication.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, x-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In addition, it also is contemplated that immunotherapy, hormone therapy, toxin therapy and surgery can be used.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### III. EXAMPLES

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 1—Materials and Methods

Ethics Statement.

All clinical data and patient samples were collected following approval by the Massachusetts General Hospital institutional review board and the Hospital of the University of Pennsylvania institutional review board. In all cases informed consent was obtained. All animal studies were conducted in accordance with the Guide for the Care and Use of Laboratory Animals of the NIH. Mice were maintained according to the guidelines of the Wistar Institutional Animal Care and Use Committee (IACUC), and study designs were approved by the Wistar IACUC.

Cell Lines and Short-Term Primary Cultures.

All normal skin epidermal melanocytes, keratinocytes and human metastatic melanoma cell lines that were established at The Wistar Institute have been documented in world-wide-web at wistar.org/lab/meenhard-herlyn-dvm-dsc/page/resources. UACC-62 and UACC-903 cells were kind gifts from Dr. Marianne B. Powell (Stanford University, Stanford, Calif. 94305, USA). A375 cells were purchased from ATCC. LOX-IMVI cells were kindly provided by Dr. Lin Zhang (University of Pennsylvania, Philadelphia, Pa. 19104, USA). All resistant cell lines that acquired drug resistance to PLX4720 or GSK2118436 (hereafter referred to as “BR” cell lines) or the combination of PLX4720 and PD0325901 (hereafter referred to as “CR” cell lines) were established after continuous exposure to PLX4720 at 10 μM, GSK2118436 at 1 μM or the combination of PLX4720 at 10 μM and PD0325901 at 1 μM. 499 and JB2 cells were kind gifts from Dr. Andy Minn (University of Pennsylvania, Philadelphia, Pa. 19104, USA). Fine needle aspiration (FNA) tumor samples derived from melanoma patients were directly transplanted and grown in mice. Tumors were harvested, fragmented and re-transplanted in mice to establish melanoma patient-derived xenografts (PDX). Short-term primary cultures established from resistant PDXs (hereafter referred to as “RPDX” cell lines) were maintained in the presence of PLX4720 at 1 μM. Human serous ovarian cancer cell lines PEO1, PEO4, PEO1-CR, WO-24-2 were established and cultured as previously described (George et al., 2017; Kim et al., 2016). PEO1 and PEO4 were kind gifts from Dr. Andrew Godwin (University of Kansas, Lawrence, Kans., USA). Specifically, PEO1-CR cells were generated by long-term treatment of PEO1 cells with Carboplatin at 3-15 μM over 10 months (Hospira, Lake Forest, Ill.). The WO-24-2 primary ovarian culture was generated from a patient with high grade serous ovarian cancer (HGSOC) and the cells were cultured in OCMI-E media (Live Tumor Culture Core at Sylvester Comprehensive Cancer Center, Miller School of Medicine, University of Miami). Except WO-24-2, all cell lines were maintained in RPMI-1640 media (Mediatech, Inc.) supplemented with 10% fetal bovine serum (Tissue Culture Biologicals) and cultured in a 37° C. humidified incubator supplied with 5% CO₂. All cell lines were authenticated by DNA fingerprinting.

Chemicals.

The BRAF inhibitor PLX4720 was provided by Plexxikon Inc. The BRAF inhibitor GSK2118436 and the MEK inhibitor PD0325901 were purchased from Selleckchem. 6-thio-dG used for in vitro studies was purchased from Metkinen Chemistry Oy. 6-thio-dG used for in vivo studies was purchased from R I Chemical Inc (Orange, Calif. 92868, USA).

Melanoma Xenotransplantation and In Vivo Studies.

100,000 melanoma cells were harvested from cell culture and re-suspended in culture medium and Matrigel at a 1:1 ratio. Cells were subcutaneously injected into mice, which were treated with indicated inhibitors when the tumor volume reached 100 mm³. Mice were sacrificed at the end time point and solid tumors were collected. All animal experiments were performed in accordance with Wistar IACUC protocol 112330 in NOD.Dg-Prkdc scidlL2rg tm 1 Wjl/SzJ mice.

DNA Purification, Library Preparation, and Sequencing.

DNA purification was done using the DNeasy Blood & Tissue Kit (Qiagen). Fine hundred ng of genomic DNA was sheared randomly into 200 bp fragments with the Covaris™ S200 UltraSonicator (Covaris®). Sheared DNA was A-tailed and ligated with adaptor-embedded indexes using the NEBNext® Ultra™ DNA Library Prep Kit for Illumina® (New England BioLabs, Inc.). DNA quality, fragment size, and concentration of library preps were measured using Agilent's DNA 1000 chips in conjunction with the 2100 Bioanalyzer (Agilent Technologies). Samples were equimolarly pooled prior to capture with a 2.2 Mbp SureSelect^{XT} Custom Target Enrichment Kit (Agilent Technologies) targeting 108 genes previously implicated in melanomagenesis. Paired-end sequencing was performed on the HiSeq™ 2000 sequencing system (Illumina) at the Perelman School of Medicine Next-Generation Sequencing Core Facility.

Mutational Analysis.

Short-sequenced reads were aligned to the hg19 human reference genome using the Burrows-Wheeler Alignment (BWA) tool. Duplicate reads were removed, as well as reads that map to more than one location, off-target reads, and variants annotated with the incorrect transcript. The Genome Analysis Toolkit (GATK) was used for data quality assurance as well as for Single Nucleotide Variant (SNV) and small insertion and deletion (indel) calling. After downsampling by GATK, a mean target coverage of 197× was achieved. Variants were annotated with wANNOVAR.

Copy Number Variation Prediction.

Prediction of copy number variation from sequencing data was done using CODEX. This algorithm normalizes the data using a Poisson latent factor model that removes biases due to GC content, exon capture, amplification efficiency, and latent systemic artifacts. Six latent factors were used for the normalization of the dataset in this study. Segmentation was restricted to exons only for all genes. Only homozygous loss and high amplification calls are reported. Log₂ ratio thresholds used for high amplification and homozygous loss were 1.33 (copy number five) and −1.2, respectively. Visual confirmation of CNV calls was done in Nexus 7.5 (BioDiscovery, Inc.) software.

RNA Purification, Library Preparation, and Sequencing.

RNA purification was done using the AllPrep DNA/RNA Mini Kit (Qiagen) for 31 tumor biopsy specimens. The first batch of 17 RNA samples were ribo-zero treated and then subject to library preparation using Epicentre's ScriptSeq Complete Gold kit. Quality check was done on the Bioanalyzer using the High Sensitivity DNA kit and quantification was carried out using KAPA Quantification kit. Samples were sequenced on Illumina's NextSeq500 with the 2×75 bp high output in the Genomics Core Facility at The Wistar Institute. The second batch of 14 RNA samples purified from tumor biopsy specimens along and the third batch of 12 RNA samples purified from A375 and LOX-IMVI BR cells were sequenced at Broad Institute to achieve the high coverage of 50M pairs. Briefly, the Tru-Seq Non-Strand Specific RNA Sequencing which includes plating, poly-A selection and non-strand specific cDNA synthesis, library preparation, sequencing, and sample identification QC check (when Sample Qualification of a matching DNA sample is chosen).

FACS Analysis of Apoptosis and Cell Death.

Adherent cells were harvested with 0.05% Trypsin-EDTA, pooled with floating cells and then washed once with 1×DPBS. Cells were then pelleted and stained with PSVue® 643 at 0.5 μM and Propidium iodide at 50 ug/ml diluted in TES buffer for 5 min in the dark. Cells were then immediately subjected to FACS analysis using a BD LSR II flow cytometer and at least 5,000 cells per sample were acquired.

Assessment of Cell Clonogenicity.

Cells were seeded into 12-well tissue culture plates at a density of 500 cells/well as biological triplicates in drug-free medium. Medium was refreshed every 3 or 4 days for 14 days. Colonies were then stained overnight with methanol containing 0.05% crystal violet. After extensive washing with distilled H₂O, cells were air-dried and subjected to image acquisition using a Nikon D200 DLSR camera.

Gene Expression Microarray Data and RNA-Seq Data from CCLE Melanoma Cell Lines and TCGA Melanoma Patients.

Normalized CCLE gene expression microarray data were directly downloaded from Broad CCLE (http://www.broadinstitute.org/ccle). Normalized RNA-seq data of TCGA melanoma patients were downloaded from TCGA Data Portal (//tcga-data.nci.nih.gov/tcga/). Single sample gene set enrichment analysis (ssGSEA) was performed in each data set to calculate an enrichment score of each gene set.

Gene Expression Microarray and RNA-Seq Data from PDX and Relapsed Melanoma Patients.

Gene expression microarray data and RNA-sequencing data of paired pre- and post-treatment tumor biopsies derived from melanoma patients were downloaded from GEO under accession number GSE50509, GSE50535, GSE61992 and GSE65185; patient RNA-seq data from the Tirosh I et al. data set were downloaded directly from //science.sciencemag.org/content/352/6282/189.long (Tirosh et al., 2016); RNA-seq data for patients treated with ipilimumab were deposited at The cBioPortal under the study name Metastatic Melanoma (MSKCC Cell, 2015). Data were normalized, background-corrected and summarized using the R package “lumi”.

Analysis of Gene Expression Microarray Data and Reverse Phase Protein Array (RPPA) Data.

The raw data of gene expression microarrays generated from Illumina Chips were normalized, background-corrected and summarized using the R package “lumi”. Probes below background level (detection P-value<0.01) were excluded and differential expression was identified with Bayes-adjusted variance analysis using the Bioconductor Limma package. To reduce false positives, the unexpressed probes were removed. The R package “limma” was employed for gene differential expression analysis, followed by multiple test correction by the Benjamini and Hochberg procedure. Genes with adjusted p values<0.05 and fold change>2 were claimed as significantly differentially expressed and were subjected to the hypergeometric test for gene set enrichment analysis (GSEA). The inventors also conducted GSEA as previously reported. For GSEA, they analyzed gene sets obtained from the Molecular Signatures Database (world-wide-web at broadinstitute.org/gsea/msigdb/). The same differential expression analysis method was applied to RPPA data.

Kaplan-Meler Survival Analysis of TCGA Melanoma Patient Data.

The inventors clustered TCGA melanoma patient RNA-seq data into 2 groups using Cox regression analysis based on expression of two telomere transcriptional gene signatures. They then performed a log-rank test to test the survival rate difference between these subgroups.

Western Blotting and Antibodies.

Cells were washed with ice-cold PBS containing 100 μM Na₃VO₄ and scraped off culture dishes. After centrifugation, cell pellets were lysed in buffer containing 10 mM Tris-HCl, pH 7.8, 150 mM NaCl, 1 mM EDTA, 1% Nonidet P-40, 1 mM Na₃VO₄ and protease inhibitors (Roche complete protease inhibitor tablets). Lysates were cleared by micro-centrifugation and protein concentrations were determined with Protein Assay Dye Reagent Concentrate (Bio-Rad). For western blots, 20 μg of each lysate were run on 8% SDS-PAGE gels and transferred onto nitrocellulose membranes using a dye fast Trans-Blot® Turbo™ transfer system (Bio-Rad). Blots were blocked in SEA BLOCK Blocking Buffer (Thermo Scientific) diluted with IX TBS at 1:1 ratio at room temperature for 1 h, incubated overnight at 1:1000 dilutions with primary antibodies (anti-AXL: Bethyl Laboratories; anti-β-actin: Sigma; all other antibodies were purchased from Cell Signaling Technologies) at 4° C., stained with secondary antibodies conjugated to IRDye® Infrared Dyes (LI-COR Biosciences) and then visualized using an Odyssey flatbed scanner (LI-COR Biosciences).

Telomere Dysfunction Induced Foci (TIF) Assay.

The TIF assay is based on the co-localization detection of DNA damage by an antibody against gamma-H2AX, and telomeres using FITC-conjugated telomere sequence (TrAGGG)₃-specific peptide nucleic acid (PNA) probe. Briefly, LOX-IMVI-BR cells were seeded to 6-well plate (50,000 cells/well). After cells adhered to the surface (next day), 6-thio-dG was added with fresh medium. Cells were treated with or without 6-thio-dG at 5 μM every two days for 4 days. Then cells were harvested and cell numbers were counted. 100,000 cells were re-plated in 4-well chamber slides. After cells adhered to the chamber slide (next day), cells were rinsed twice with 1×PBS and fixed in 4% paraformaldehyde in PBS for 10 minutes. Cells were washed twice with PBS and permeabilized in 0.5% Triton X-100 in PBS for 10 minutes. Following permeabilization, cells were washed three times with PBS. Cells were blocked with 0.2% Fish gelatin and 0.5% BSA in PBS for 30 minutes. Gamma-H₂AX (mouse) (Millipore, Billerica, Mass.) was diluted 1:1000 in blocking solution and incubated with cells for 2 hrs. Following three washes with PBST (1×PBS in 0.1% Triton) and 3 washes with PBS, cells were incubated with Alexaflour 568 conjugated goat anti mouse (1:500) (Invitrogen, Grand Island, N.Y.) for 40 minutes, then were washed five times with 0.1% PBST. Cells were fixed in 4% paraformaldehyde in PBS for 20 minutes at room temperature. The slides were sequentially dehydrated with 70%, 90%, 100% ethanol. Following dehydration, denaturation was conducted with hybridization buffer containing FITC-conjugated telomere sequence (TrAGGG)₃-specific peptide nucleic acid (PNA) probe (PNA Bio, Thousand Oaks, Calif.), 70% formamide, 30% 2×SSC, 10% (w/v) MgCl₂.6H₂O (Fisher Sci), 0.25% (w/v) blocking reagent for nucleic acid hybridization and detection (Roche) for 7 minutes at 80′C on heat block, followed by overnight incubation at room temperature. Slides were washed sequentially with 70% formamide (Ambion, Life Technologies, Grand Island, N.Y.)/0.6×SSC (Invitrogen) (2×1 hr), 2×SSC (1×15 minutes), PBS (1×5 minutes) and sequentially dehydrated with 70%, 90%, 100% ethanol, then mounted with Vectashield mounting medium with DAPI (Vector Laboratories, Burlingame, Calif.). Images were captured with Deltavision wide-field microscope using the 60× objective, then deconvoluted using Autoquant X3. Gamma-H2AX and TIFs were quantified using Imaris software.

SA-β-gal Staining.

Cells were fixed with DPBS containing 2% formaldehyde (Sigma) and 0.2% glutaraldehyde (Sigma) for 30 minutes. Fixed cells were then incubated at 37° C. (no CO₂) with fresh SA-β-gal staining solution overnight. Images were acquired with Nikon TE2000 Inverted Microscope.

Telomere Length Quantification.

Telomere length was measured by the ratio of the telomeric DNA and a single copy gene, 36B4. The forward and reverse primers used for amplifying telomeric DNA were tel1b and tel2b. The quantification of telomeric DNA and 36B4 was determined by quantitative real time PCR on Applied Biosystems 7500 Fast Real-Time PCR System. The reaction mixtures (20 μL final volume) contained 10 μL Fast SYBR® Green Master Mix, 500 nM each primer and 10 ng genomic DNA. The reaction conditions were 95° C. for 20 s, followed by 40 cycles of 95° C. for 3 s and 60° C. for 30 s. The telomere length was analyzed by 2^{ΔΔCt} method.

Patients' cDNA Samples and Quantitative Real-Time PCR.

Total RNA was purified from patients' tumor biopsies according to the manufacture's instruction (RNeasy Mini Kit, Qiagen). One μg total mRNA was reverse transcribed using a Maxima First Strand cDNA Synthesis Kit for qRT-PCR (Thermo Fisher). Fast SYBR® Green Master Mix (Life Technologies) was used with cDNA template and primers to evaluate the expression of TERT and GAPDH. Primers used were purchased from Integrated DNA Technologies. Amplifications were performed using an Applied Biosystems® 7500 Real-Time PCR System (Life Technologies). All experiments were performed in triplicate. Expression ratios of controls were normalized to 1. Please see Extended Experimental Procedures for the list of PCR primer sequences.

Statistics.

Unless otherwise indicated, data in the figures were presented as mean±SEM for 3 biological or technical replicates. Significant differences between experimental conditions were determined using the two-tailed unpaired t test. For survival data, Kaplan-Meier survival curves were generated and their differences were examined with Log-rank test. For tumor growth data, mixed effect models were used to determine the differences between treatment groups in tumor volume change at the end of experiment. A two-sided p value of less than 0.05 was considered statistically significant. *: p<0.05; **: p<0.005; ***: p<0.0005.

### Example 2—Results

Treatment of a Variety of Cancer Cell Lines with Telomerase-Directed 6-Thio-dG Impairs Cell Viability.

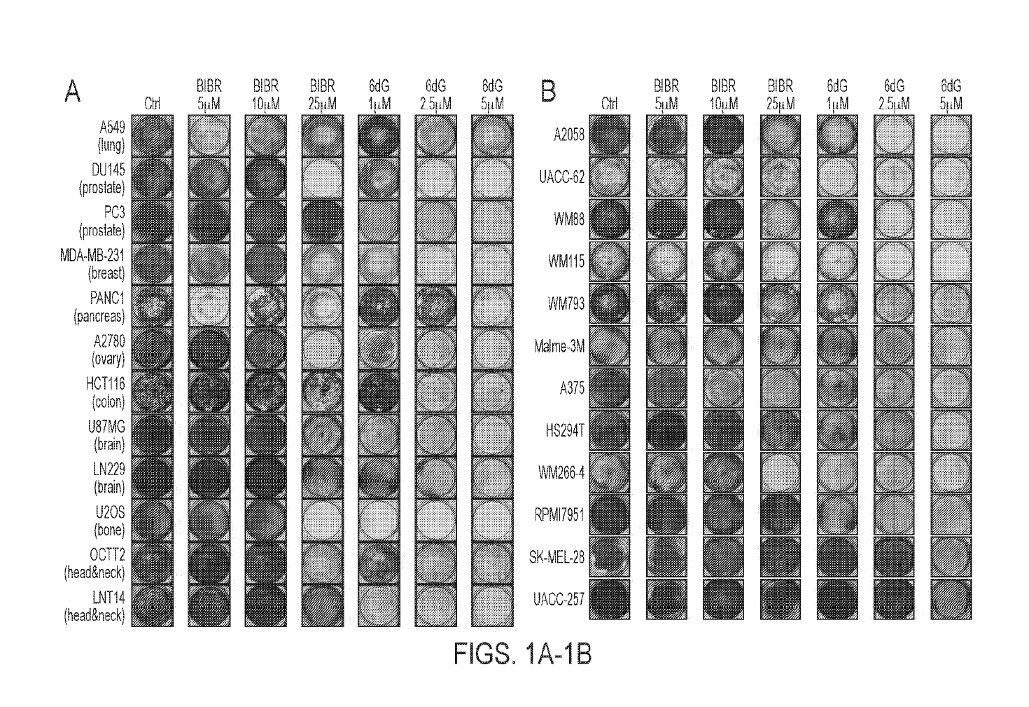

It has previously been shown that 6-thio-dG inhibited cell viability of the colon cancer cell line, HCT-116 and the non-small cell lung cancer cell line, A549 (Mender et al., 2015). To further confirm the inhibitory effect of 6-thio-dG, a panel of 12 cancer cell lines of 9 different histological origins was treated with 6-thio-dG for 9-12 days. As the control for 6-thio-dG, a known telomerase inhibitor, BIBR 1532, was also included. In most cases, these cancer cell lines were sensitive to 6-thio-dG administered at a dose of 2.5 μM and higher (FIG. 1A). The anti-proliferative activity of BIBR 1532 was also observed in a subset of 12 cancer cell lines that were administered at a dose of 25 μM (FIG. 1A).

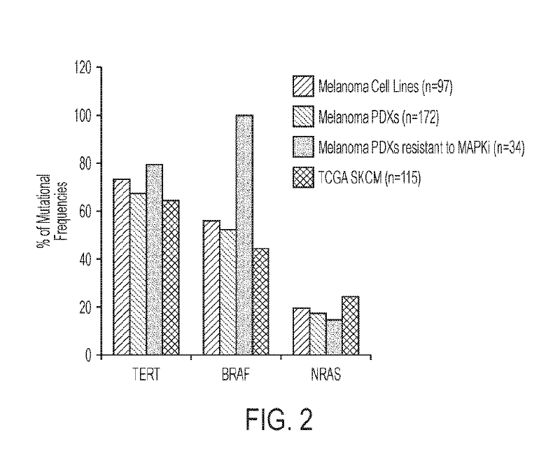

The TERT promoter is often mutated in human cancers including melanoma. Massively parallel sequencing (MPS) of 108 genes that are implicated in melanomagenesis was conducted (Table 1). Collectively, various TERT promoter mutations (Chr5:1295161 T>G; Chr5:1295228 G>A; Chr5:1295228-1295229 GG>AA; Chr5:1295242 G>A; Chr5:1295242-1295243 GG>AA; and Chr5:1295250 G>A) were identified in 73.2% of 97 melanoma cell lines, 67.4% of 172 melanoma therapy-naïve patient-derived xenografts (PDX), and 79.4% of 34 melanoma PDX that acquired resistance to MAPKi (FIG. 2; Tables 2-4). These observations are in line with sequencing data from the Cancer Genome Atlas (TCGA) showing that 64.3% of 115 cases of skin cutaneous melanoma (SKCM) harbor mutations in the TERT promoter (FIG. 2; Table 5). The frequency of TERT promoter mutations in melanoma cell lines, melanoma PDXs and TCGA SKCM is very similar to that of BRAF^{v600} mutations (FIG. 2). Not surprisingly, TERT was the second most frequently mutated gene after BRAF in the MAPKi-resistant PDX data set. Thus, the results confirm and further highlight the prevalence of TERT promoter mutations in both therapy-naïve and therapy-resistant melanomas.

TABLE 1
The list of 108 genes included in massively parallel sequencing.

| | Exonic | | |
|---|---|---|---|
| Full Gene | Regions | | |
| APC | ABL1 | GRM3 | RAC1 |
| ARID1A | AKT1 | HRAS | RAC2 |
| ARID2 | AKT3 | IDH1 | RET |
| ATM | ALK | IDH2 | RHOT1 |
| BAP1 | ARID1B | JAK3 | SETD2 |
| BRAF | CASP8 | KDR | SMARCA4 |
| BRCA1 | CCND1 | KIT | SMO |
| BRCA2 | CCND2 | KRAS | SNX31 |
| CDC42 | CDK4 | MAP2K1 | SOX10 |
| CDKN2A | CDK6 | MAP2K2 | SRC |
| CDKN2B | CTNNB1 | MAP3K5 | STK19 |
| EIF2AK3 | CXCR4 | MAP3K9 | STK24 |
| EZH1 | DCC | MDM2 | TACC1 |
| EZH2 | DDX3X | MDM4 | TRRAP |
| MLH1 | DYNC1I1 | MET | VAV3 |
| NF1 | EGFR | MITF | |
| NF2 | ERBB2 | MMP8 | |
| NTRK1 | ERBB3 | MYC | |
| PIK3CA | ERBB4 | NOTCH1 | |
| PRDM1 | FBXO4 | NOTCH2 | |
| PRDM2 | FBXW7 | NRAS | |
| PTEN | FGF3 | PDGFRA | |
| RB1 | FGF4 | PDGFRB | |
| ROS1 | FGFR2 | PIK3CB | |
| SMAD4 | FGFR3 | PPP6C | |
| SMARCB1 | FGFR4 | PRC1 | |
| STK11 | FLT3 | PREX1 | |
| TERT | GNA11 | PREX2 | |
| TP53 | GNAQ | PTCH1 | |
| VHL | GNAS | PTPRD | |
| WT1 | GRIN2A | PTPRK | |

TABLE 2
TERT promoter mutations in Wistar's melanoma cell lines.

| | | | | | | | Second TERT |
|---|---|---|---|---|---|---|---|
| | Sample | | | TERT Promoter | TERT Promoter | Second TERT | Promoter |
| Sample | Type | Sub-type | BRAF | Mutation | Mutation Call | Promoter Mutation | Mutation Call |
| Skmel28p93 | Cell | BRAF | BRAF | TERT Chr5: 1295161 T > G | VUS | | |
| | Line | Hotspot- | V600E | | | | |
| | | Mutant | | | | | |
| ND238p5 | Cell | BRAF | BRAF | TERT Chr5: 1295228 G > A | Deleterious | | |
| | Line | Hotspot- | V600E | | | | |
| | | Mutant | | | | | |
| WM1716p10 | Cell | BRAF | BRAF | TERT Chr5: 1295228 G > A | Deleterious | | |
| | Line | Hotspot- | V600E | | | | |
| | | Mutant | | | | | |
| WM1799p11 | Cell | BRAF | BRAF | TERT Chr5: 1295228 G > A | Deleterious | | |
| | Line | Hotspot- | V600E | | | | |
| | | Mutant | | | | | |
| WM3248p35 | Cell | BRAF | BRAF | TERT Chr5: 1295228 G > A | Deleterious | | |
| | Line | Hotspot- | V600E | | | | |
| | | Mutant | | | | | |
| WM3301p2+ | Cell | BRAF | BRAF | TERT Chr5: 1295228 G > A | Deleterious | | |
| | Line | Hotspot- | V600E | | | | |
| | | Mutant | | | | | |
| WM793p61 | Cell | BRAF | BRAF | TERT Chr5: 1295228 G > A | Deleterious | | |
| | Line | Hotspot- | V600E | | | | |
| | | Mutant | | | | | |
| WM858p39 | Cell | BRAF | BRAF | TERT Chr5: 1295228 G > A | Deleterious | | |
| | Line | Hotspot- | V600E | | | | |
| | | Mutant | | | | | |
| WM873-1 | Cell | BRAF | BRAF | TERT Chr5: 1295228 G > A | Deleterious | | |
| | Line | Hotspot- | V600E | | | | |
| | | Mutant | | | | | |
| WM983Cp43 | Cell | BRAF | BRAF | TERT Chr5: 1295228 G > A | Deleterious | | |
| | Line | Hotspot- | V600E | | | | |
| | | Mutant | | | | | |
| WM3172p17 | Cell | BRAF | BRAF | TERT Chr5: 1295242 G > A | VUS | | |
| | Line | Hotspot- | V600E | | | | |
| | | Mutant | | | | | |
| WM3704p24 | Cell | BRAF | BRAF | TERT Chr5: 1295250 G > A | Deleterious | | |
| | Line | Hotspot- | V600E | | | | |
| | | Mutant | | | | | |
| WM88p49 | Cell | BRAF | BRAF | TERT Chr5: 1295250 G > A | Deleterious | | |
| | Line | Hotspot- | V600E | | | | |
| | | Mutant | | | | | |
| WM989 | Cell | BRAF | BRAF | TERT Chr5: 1295250 G > A | Deleterious | | |
| | Line | Hotspot- | V600E | | | | |
| | | Mutant | | | | | |
| 451Lu- | Cell | BRAF | BRAF | TERT Chr5: 1295250 G > A | Deleterious | | |
| C2-MR | Line | Hotspot- | V600E | | | | |
| | | Mutant | | | | | |
| WM1232p94 | Cell | BRAF | BRAF | TERT Chr5: 1295250 G > A | Deleterious | | |
| | Line | Hotspot- | V600E | | | | |
| | | Mutant | | | | | |
| WM164p127 | Cell | BRAF | BRAF | TERT Chr5: 1295250 G > A | Deleterious | | |
| | Line | Hotspot- | V600E | | | | |
| | | Mutant | | | | | |
| WM853- | Cell | BRAF | BRAF | TERT Chr5: 1295242-1295243 | Deleterious | | |
| 2p36 | Line | Hotspot- | V600E | GG > AA | | | |
| | | Mutant | | | | | |
| WM983B- | Cell | BRAF | BRAF | TERT Chr5: 1295228 G > A | Deleterious | | |
| BR | Line | Hotspot- | V600E | | | | |
| | | Mutant | | | | | |
| WM1727A | Cell | BRAF | BRAF | TERT Chr5: 1295228 G > A | Deleterious | | |
| | Line | Hotspot- | V600E | | | | |
| | | Mutant | | | | | |
| WM3482p31 | Cell | BRAF | BRAF | TERT Chr5: 1295228 G > A | Deleterious | | |
| | Line | Hotspot- | V600E | | | | |
| | | Mutant | | | | | |
| WM1158p14+ | Cell | BRAF | BRAF | TERT Chr5: 1295250 G > A | Deleterious | | |
| | Line | Hotspot- | V600E | | | | |
| | | Mutant | | | | | |
| WM2090p15 | Cell | BRAF | BRAF | TERT Chr5: 1295250 G > A | Deleterious | | |
| | Line | Hotspot- | V600E | | | | |
| | | Mutant | | | | | |
| WM1026p22 | Cell | BRAF | BRAF | TERT Chr5: 1295228 G > A | Deleterious | | |
| | Line | Hotspot- | V600E | | | | |
| | | Mutant | | | | | |
| WM3533p13 | Cell | BRAF | BRAF | TERT Chr5: 1295228 G > A | Deleterious | | |
| | Line | Hotspot- | V600E | | | | |
| | | Mutant | | | | | |
| WM373p31 | Cell | BRAF | BRAF | TERT Chr5: 1295228 G > A | Deleterious | | |
| | Line | Hotspot- | V600E | | | | |
| | | Mutant | | | | | |
| WM75p17 | Cell | BRAF | BRAF | TERT Chr5: 1295228 G > A | Deleterious | | |
| | Line | Hotspot- | V600E | | | | |
| | | Mutant | | | | | |
| WM983Ap5 | Cell | BRAF | BRAF | TERT Chr5: 1295228 G > A | Deleterious | | |
| | Line | Hotspot- | V600E | | | | |
| | | Mutant | | | | | |
| WM9p68 | Cell | BRAF | BRAF | TERT Chr5: 1295228 G > A | Deleterious | | |
| | Line | Hotspot- | V600E | | | | |
| | | Mutant | | | | | |
| M331p16 | Cell | BRAF | BRAF | TERT Chr5: 1295228 G > A | Deleterious | TERT | Deleterious |
| | Line | Hotspot- | V600E | | | Chr5: 1295250 G > A | |
| | | Mutant | | | | | |
| WM1819p4 | Cell | BRAF | BRAF | TERT Chr5: 1295228-1295229 | Deleterious | | |
| | Line | Hotspot- | V600E | GG > AA | | | |
| | | Mutant | | | | | |
| WM2044p12 | Cell | BRAF | BRAF | TERT Chr5: 1295242-1295243 | Deleterious | | |
| | Line | Hotspot- | V600E | GG > AA | | | |
| | | Mutant | | | | | |
| WM1985p4 | Cell | BRAF | BRAF | TERT Chr5: 1295250 G > A | Deleterious | | |
| | Line | Hotspot- | V600E | | | | |
| | | Mutant | | | | | |
| WM902Bp11 | Cell | BRAF | BRAF | TERT Chr5: 1295250 G > A | Deleterious | | |
| | Line | Hotspot- | V600E | | | | |
| | | Mutant | | | | | |
| LH6215p7 | Cell | BRAF | BRAF | TERT Chr5: 1295250 G > A | Deleterious | TERT | VUS |
| | Line | Hotspot- | V600E | | | chr5: 1295172-1295173 | |
| | | Mutant | | | | CC > TT | |
| WM3630p5 | Cell | BRAF | BRAF | TERT Chr5: 1295250 G > A | Deleterious | TERT chr5: 1295205 | VUS |
| | Line | Hotspot- | V600E | | | G > A | |
| | | Mutant | | | | | |
| WM3282p20 | Cell | BRAF | BRAF | TERT Chr5: 1295250 G > A | Deleterious | TERT chr5: 1295205 | VUS |
| | Line | Hotspot- | V600K | | | G > A | |
| | | Mutant | | | | | |
| WM115 | Cell | BRAF | Other | TERT Chr5: 1295250 G > A | Deleterious | | |
| | Line | Hotspot- | BRAF | | | | |
| | | Mutant | Hotspot | | | | |
| WM1341Dp37 | Cell | BRAF | Other | TERT Chr5: 1295250 G > A | Deleterious | | |
| | Line | Hotspot- | BRAF | | | | |
| | | Mutant | Hotspot | | | | |
| WM239Ap41 | Cell | BRAF | Other | TERT Chr5: 1295250 G > A | Deleterious | | |
| | Line | Hotspot- | BRAF | | | | |
| | | Mutant | Hotspot | | | | |
| WM266-4 | Cell | BRAF | Other | TERT Chr5: 1295250 G > A | Deleterious | | |
| | Line | Hotspot- | BRAF | | | | |
| | | Mutant | Hotspot | | | | |
| WM165- | Cell | BRAF | Other | TERT Chr5: 1295250 G > A | Deleterious | | |
| 1p212 | Line | Hotspot- | BRAF | | | | |
| | | Mutant | Hotspot | | | | |
| WM1361Ap40 | Cell | RAS | | TERT Chr5: 1295228 G > A | Deleterious | | |
| | Line | Hotspot- | | | | | |
| | | Mutant | | | | | |
| WM3506p33 | Cell | RAS | | TERT Chr5: 1295228 G > A | Deleterious | | |
| | Line | Hotspot- | | | | | |
| | | Mutant | | | | | |
| WM3619p10 | Cell | RAS | | TERT Chr5: 1295228 G > A | Deleterious | | |
| | Line | Hotspot- | | | | | |
| | | Mutant | | | | | |
| WM2032 | Cell | RAS | | TERT Chr5: 1295228 G > A | Deleterious | TERT | Deleterious |
| | Line | Hotspot- | | | | Chr5: 1295242-1295243 | |
| | | Mutant | | | | GG > AA | |
| WM3451 | Cell | RAS | | TERT Chr5: 1295242-1295243 | Deleterious | | |
| | Line | Hotspot- | | GG > AA | | | |
| | | Mutant | | | | | |
| WM3268p13 | Cell | RAS | | TERT Chr5: 1295250 G > A | Deleterious | | |
| | Line | Hotspot- | | | | | |
| | | Mutant | | | | | |
| WM3406p2+ | Cell | RAS | | TERT Chr5: 1295250 G > A | Deleterious | | |
| | Line | Hotspot- | | | | | |
| | | Mutant | | | | | |
| WM3702 | Cell | RAS | | TERT Chr5: 1295250 G > A | Deleterious | | |
| | Line | Hotspot- | | | | | |
| | | Mutant | | | | | |
| WM3758p19 | Cell | RAS | | TERT Chr5: 1295250 G > A | Deleterious | | |
| | Line | Hotspot- | | | | | |
| | | Mutant | | | | | |
| WM3854p12 | Cell | RAS | | TERT Chr5: 1295250 G > A | Deleterious | | |
| | Line | Hotspot- | | | | | |
| | | Mutant | | | | | |
| WM1366p60 | Cell | RAS | | TERT Chr5: 1295250 G > A | Deleterious | | |
| | Line | Hotspot- | | | | | |
| | | Mutant | | | | | |
| WM1960p10 | Cell | RAS | | TERT Chr5: 1295250 G > A | Deleterious | | |
| | Line | Hotspot- | | | | | |
| | | Mutant | | | | | |
| WM2013p36 | Cell | RAS | | TERT Chr5: 1295250 G > A | Deleterious | | |
| | Line | Hotspot- | | | | | |
| | | Mutant | | | | | |
| WM3060 | Cell | RAS | | TERT Chr5: 1295250 G > A | Deleterious | | |
| | Line | Hotspot- | | | | | |
| | | Mutant | | | | | |
| WM3623p15 | Cell | RAS | | TERT Chr5: 1295250 G > A | Deleterious | TERT | VUS |
| | Line | Hotspot- | | | | chr5: 1295230-1295231 | |
| | | Mutant | | | | GG > AA | |
| M230p6 | Cell | RAS | BRAF | TERT Chr5: 1295228-1295229 | Deleterious | TERT Chr5: 1295230 | VUS |
| | Line | Hotspot- | V600K | GG > AA | | G > A | |
| | | Mutant | | | | | |
| WM209p11 | Cell | RAS | | TERT Chr5: 1295228 G > A | Deleterious | | |
| | Line | Hotspot- | | | | | |
| | | Mutant | | | | | |
| WM1791Cp14+ | Cell | RAS | | TERT Chr5: 1295250 G > A | Deleterious | | |
| | Line | Hotspot- | | | | | |
| | | Mutant | | | | | |
| WM3211 | Cell | KIT-Mutant | | TERT Chr5: 1295250 G > A | Deleterious | | |
| | Line | | | | | | |
| WM3912p9 | Cell | NF1-Mutant | Non- | TERT Chr5: 1295228 G > A | Deleterious | | |
| | Line | | BRAF | | | | |
| | | | Hotspot | | | | |
| WM3622p18 | Cell | NF1-Mutant | | TERT Chr5: 1295250 G > A | Deleterious | TERT | VUS |
| | Line | | | | | Chr5: 1295253 G > A | |
| WM1963p21 | Cell | NF1-Mutant | Non- | TERT Chr5: 1295250 G > A | Deleterious | | |
| | Line | | BRAF | | | | |
| | | | Hotspot | | | | |
| TH202p14 | Cell | Wildtype | | TERT Chr5: 1295228 G > A | Deleterious | | |
| | Line | | | | | | |
| WM3311p6+ | Cell | Wildtype | | TERT Chr5: 1295228 G > A | Deleterious | TERT chr5: 1295238 | VUS |
| | Line | | | | | C > T | |
| WM3438p6 | Cell | Wildtype | | TERT Chr5: 1295250 G > A | Deleterious | | |
| | Line | | | | | | |
| WM3918p82 | Cell | Wildtype | | TERT Chr5: 1295250 G > A | Deleterious | | |
| | Line | | | | | | |
| WM3246p17 | Cell | Wildtype | | TERT Chr5: 1295228 G > A | Deleterious | TERT | VUS |
| | Line | | | | | Chr5: 1295253-1295254 | |
| | | | | | | GG > AA | |
| WM3928- | Cell | Wildtype | | TERT Chr5: 1295228 G > A | Deleterious | | |
| 2p3 | Line | | | | | | |
| WM3743p24 | Cell | Wildtype | | TERT Chr5: 1295250 G > A | Deleterious | | |
| | Line | | | | | | |

TABLE 3
TERT promoter mutations in Wistar's treatment-naïve PDX

| | Sample | | | | | | TERT | Second | Second |
|---|---|---|---|---|---|---|---|---|---|
| | Type R- | | | | | TERT | Promoter | TERT | TERT |
| | plot | sub- | | | | Promoter | Mutation | Promoter | Promoter |
| Sample | Code | type | BRAF | KRAS | NRAS | Mutation | Call | Mutation | Mutation Call |
| CS190MP2 | PDX | BRAF | BRAF | | | TERT | Delete- | | |
| | | Hotspot- | V600K | | | Chr5: 1295228 | rious | | |
| | | Mutant | | | | G > A | | | |
| RC6041MP1 | PDX | RAS | | | NRAS | TERT | Delete- | | |
| MouseNum1365 | | Hotspot- | | | 12/3 | Chr5: 1295228 | rious | | |
| | | Mutant | | | | G > A | | | |
| RR214MP1 | PDX | RAS | | | NRAS | TERT | Delete- | | |
| MouseNum5171 | | Hotspot- | | | Codon 61 | Chr5: 1295228 | rious | | |
| | | Mutant | | | | G > A | | | |
| WM3703MP1 | PDX | RAS | | | NRAS | TERT | Delete- | | |
| MouseNum8244 | | Hotspot- | | | Codon 61 | Chr5: 1295228 | rious | | |
| | | Mutant | | | | G > A | | | |
| WM3903- | PDX | BRAF | BRAF | | | TERT | VUS | | |
| 1MP5MouseNum4045 | | Hotspot- | V600E | | | Chr5: 1295242 | | | |
| | | Mutant | | | | G > A | | | |
| WM3903- | PDX | BRAF | BRAF | | | TERT | Delete- | | |
| 1MP6MouseNum6576 | | Hotspot- | V600E | | | Chr5: 1295228 | rious | | |
| | | Mutant | | | | G > A | | | |
| WM3908MP3 | PDX | BRAF | BRAF | | | TERT | Delete- | | |
| MouseNum1563RECUT | | Hotspot- | V600K | | | Chr5: 1295250 | rious | | |
| | | Mutant | | | | G > A | | | |
| WM3908MP4PLX | PDX | BRAF | BRAF | | | TERT | Delete- | | |
| MouseNum6433RECUT | | Hotspot- | V600K | | | Chr5: 1295250 | rious | | |
| | | Mutant | | | | G > A | | | |
| WM3909PLX2MP7 | PDX | BRAF | BRAF | | | TERT | Delete- | | |
| | | Hotspot- | V600E | | | Chr5: 1295250 | rious | | |
| | | Mutant | | | | G > A | | | |
| WM3915- | PDX | RAS | | | NRAS | TERT | Delete- | | |
| 2MP1MouseNum1337 | | Hotspot- | | | Codon 61 | Chr5: 1295228 | rious | | |
| | | Mutant | | | | G > A | | | |
| WM3921MP1 | PDX | Wildtype | | | | TERT | Delete- | | |
| | | | | | | Chr5: 1295228 | rious | | |
| | | | | | | G > A | | | |
| WM3924MP4 | PDX | RAS | | | NRAS | TERT | Delete- | | |
| MouseNum3855 | | Hotspot- | | | Codon 61 | Chr5: 1295228 | rious | | |
| | | Mutant | | | | G > A | | | |
| WM3927MP2 | PDX | BRAF | BRAF | | | TERT | Delete- | | |
| MouseNum3888 | | Hotspot- | V600K | | | Chr5: 1295228 | rious | | |
| | | Mutant | | | | G > A | | | |
| WM3931MP2 | PDX | BRAF | BRAF | | | TERT | Delete- | | |
| MouseNum3228 | | Hotspot- | V600E | | | Chr5: 1295250 | rious | | |
| | | Mutant | | | | G > A | | | |
| WM3933MP1 | PDX | BRAF | BRAF | | | TERT | VUS | | |
| MouseNum | | Hotspot- | V600E | | | Chr5: 1295242 | | | |
| | | Mutant | | | | G > A | | | |
| WM3937MP1 | PDX | RAS | | | NRAS | TERT | Delete- | | |
| | | Hotspot- | | | 12/3 | Chr5: 1295250 | rious | | |
| | | Mutant | | | | G > A | | | |
| WM3939- | PDX | BRAF | BRAF | | | TERT | VUS | | |
| 2CPLXMP6 | | Hotspot- | V600E | | | Chr5: 1295242 | | | |
| MouseNum5900 | | Mutant | | | | G > A | | | |
| WM3941- | PDX | BRAF | Other | | | TERT | Delete- | | |
| 1MP3MouseNum8890 | | Hotspot- | BRAF | | | Chr5: 1295250 | rious | | |
| | | Mutant | Hotspot | | | G > A | | | |
| WM3942CPLXMP3 | PDX | BRAF | BRAF | | | TERT | Delete- | | |
| MouseNum8885 | | Hotspot- | V600E | | | Chr5: 1295228 | rious | | |
| | | Mutant | | | | G > A | | | |
| WM3960MP4 | PDX | BRAF | BRAF | | | TERT | Delete- | | |
| MouseNum3960 | | Hotspot- | V600E | | | Chr5: 1295228 | rious | | |
| | | Mutant | | | | G > A | | | |
| WM3973MP3 | PDX | BRAF | BRAF | | | TERT | Delete- | | |
| MouseNum3470 | | Hotspot- | V600E | | | Chr5: 1295250 | rious | | |
| | | Mutant | | | | G > A | | | |
| WM3973MP4 | PDX | BRAF | BRAF | | | TERT | Delete- | | |
| MouseNum7144 | | Hotspot- | V600E | | | Chr5: 1295250 | rious | | |
| | | Mutant | | | | G > A | | | |
| WM3973PLXMP5 | PDX | BRAF | BRAF | | | TERT | Delete- | | |
| MouseNum7627 | | Hotspot- | V600E | | | Chr5: 1295250 | rious | | |
| | | Mutant | | | | G > A | | | |
| WM3983PLXMP7 | PDX | RAS | BRAF | | NRAS | TERT | Delete- | | |
| MouseNum8003 | | Hotspot- | V600K | | Codon 61 | Chr5: 1295250 | rious | | |
| | | Mutant | | | | G > A | | | |
| WM3991MP1 | PDX | Wildtype | | | | TERT | VUS | | |
| MouseNum4044 | | | | | | chr5: 1295161 | | | |
| | | | | | | T > G | | | |
| WM3994- | PDX | BRAF | BRAF | | | TERT | Delete- | | |
| 3MP2MouseNum127 | | Hotspot- | V600E | | | Chr5: 1295250 | rious | | |
| | | Mutant | | | | G > A | | | |
| WM3994MP1 | PDX | BRAF | BRAF | | | TERT | Delete- | | |
| MouseNum3661RECUT | | Hotspot- | V600E | | | Chr5: 1295250 | rious | | |
| | | Mutant | | | | G > A | | | |
| WM3997MP3 | PDX | BRAF | BRAF | | | TERT | Delete- | | |
| MouseNum8120 | | Hotspot- | V600K | | | Chr5: 1295228 | rious | | |
| | | Mutant | | | | G > A | | | |
| WM4003MP1 | PDX | Wildtype | | | | TERT | Delete- | | |
| MouseNum8866 | | | | | | Chr5: 1295228 | rious | | |
| | | | | | | G > A | | | |
| WM4008MP2 | PDX | BRAF | BRAF | | | TERT | Delete- | | |
| MouseNum4345 | | Hotspot- | V600K | | | Chr5: 1295250 | rious | | |
| | | Mutant | | | | G > A | | | |
| WM4008MP47433 | PDX | BRAF | BRAF | | | TERT | Delete- | | |
| | | Hotspot- | V600K | | | Chr5: 1295250 | rious | | |
| | | Mutant | | | | G > A | | | |
| WM4018- | PDX | BRAF | BRAF | | | TERT | VUS | | |
| 7MP1MouseNum0332 | | Hotspot- | V600K | | | Chr5: 1295242 | | | |
| | | Mutant | | | | G > A | | | |
| WM4023MP1 | PDX | RAS | | | NRAS | TERT | Delete- | | |
| MouseNum2770 | | Hotspot- | | | Codon 61 | Chr5: 1295250 | rious | | |
| | | Mutant | | | | G > A | | | |
| WM4035MP1 | PDX | BRAF | BRAF | | | TERT | Delete- | | |
| MouseNum2736 | | Hotspot- | V600E | | | Chr5: 1295250 | rious | | |
| | | Mutant | | | | G > A | | | |
| WM4037MP1 | PDX | BRAF | BRAF | | | TERT | Delete- | | |
| MouseNum3896 | | Hotspot- | V600E | | | Chr5: 1295228 | rious | | |
| | | Mutant | | | | G > A | | | |
| WM4042MP1 | PDX | RAS | | | NRAS | TERT | Delete- | | |
| MouseNum4897 | | Hotspot- | | | Codon 61 | Chr5: 1295228 | rious | | |
| | | Mutant | | | | G > A | | | |
| WM4053- | PDX | BRAF | BRAF | | | TERT | Delete- | | |
| 2MP1MouseNum5272 | | Hotspot- | V600E | | | Chr5: 1295228 | rious | | |
| | | Mutant | | | | G > A | | | |
| WM4055MP1 | PDX | BRAF | BRAF | | | TERT | Delete- | | |
| MouseNum5191 | | Hotspot- | V600K | | | Chr5: 1295228 | rious | | |
| | | Mutant | | | | G > A | | | |
| WM4070- | PDX | BRAF | BRAF | | | TERT | Delete- | | |
| 2MP4MouseNum6758 | | Hotspot- | V600K | | | Chr5: 1295228 | rious | | |
| | | Mutant | | | | G > A | | | |
| WM4071- | PDX | BRAF | BRAF | | | TERT | Delete- | | |
| 1MP2MouseNum6673 | | Hotspot- | V600E | | | Chr5: 1295250 | rious | | |
| | | Mutant | | | | G > A | | | |
| WM4071- | PDX | BRAF | BRAF | | | TERT | Delete- | | |
| 2MP2717 | | Hotspot- | V600E | | | Chr5: 1295250 | rious | | |
| | | Mutant | | | | G > A | | | |
| WM4073MP1 | PDX | NF1- | | | | TERT | Delete- | | |
| MouseNum8858 | | Mutant | | | | Chr5: 1295228 | rious | | |
| | | | | | | G > A | | | |
| WM4089MP1 | PDX | BRAF | BRAF | | | TERT | Delete- | | |
| MouseNum6553 | | Hotspot- | V600E | | | Chr5: 1295228 | rious | | |
| | | Mutant | | | | G > A | | | |
| WM4115MP2 | PDX | NF1- | | | | TERT | Delete- | | |
| MouseNum8865 | | Mutant | | | | Chr5: 1295250 | rious | | |
| | | | | | | G > A | | | |
| WM4150MP1 | PDX | BRAF | BRAF | | | TERT | Delete- | | |
| MouseNum8407 | | Hotspot- | V600K | | | Chr5: 1295250 | rious | | |
| | | Mutant | | | | G > A | | | |
| WM4205- | PDX | BRAF | Other | | | TERT | Delete- | | |
| 1MP1MouseNum6786 | | Hotspot- | BRAF | | | Chr5: 1295250 | rious | | |
| | | Mutant | Hotspot | | | G > A | | | |
| WM4205- | PDX | BRAF | Other | | | TERT | Delete- | | |
| 2PLXMP3MouseNum8835 | | Hotspot- | BRAF | | | Chr5: 1295250 | rious | | |
| | | Mutant | Hotspot | | | G > A | | | |
| WM4205- | PDX | BRAF | Other | | | TERT | Delete- | | |
| 3MP2MouseNum7681 | | Hotspot- | BRAF | | | Chr5: 1295250 | rious | | |
| | | Mutant | Hotspot | | | G > A | | | |
| WM4206MP1 | PDX | BRAF | BRAF | | | TERT | Delete- | | |
| MouseNum6761 | | Hotspot- | V600K | | | Chr5: 1295250 | rious | | |
| | | Mutant | | | | G > A | | | |
| WM4208MP1 | PDX | RAS | | | NRAS | TERT | Delete- | | |
| MouseNum6778 | | Hotspot- | | | Codon 61 | Chr5: 1295228 | rious | | |
| | | Mutant | | | | G > A | | | |
| WM4210MP1 | PDX | NF1- | | | | TERT | Delete- | | |
| MouseNum7463 | | Mutant | | | | Chr5: 1295228- | rious | | |
| | | | | | | 1295229 | | | |
| | | | | | | GG > AA | | | |
| WM4215MP1 | PDX | BRAF | BRAF | | | TERT | Delete- | | |
| MouseNum6780 | | Hotspot- | V600E | | | Chr5: 1295228 | rious | | |
| | | Mutant | | | | G > A | | | |
| WM4223MP1 | PDX | KIT- | Non- | | | TERT | Delete- | | |
| MouseNum7296 | | Mutant | BRAF | | | Chr5: 1295228 | rious | | |
| | | | Hotspot | | | G > A | | | |
| WM4224MP1 | PDX | RAS | | | NRAS | TERT | Delete- | | |
| MouseNum7476 | | Hotspot- | | | Codon 61 | Chr5: 1295250 | rious | | |
| | | Mutant | | | | G > A | | | |
| WM4225MP1 | PDX | BRAF | BRAF | | | TERT | Delete- | | |
| MouseNum7427 | | Hotspot- | V600E | | | Chr5: 1295228 | rious | | |
| | | Mutant | | | | G > A | | | |
| WM4231- | PDX | RAS | Non- | | NRAS | TERT | Delete- | | |
| 2MP1MouseNum7874 | | Hotspot- | BRAF | | Codon 61 | Chr5: 1295250 | rious | | |
| | | Mutant | Hotspot | | | G > A | | | |
| WM4235MP1 | PDX | RAS | | | NRAS | TERT | Delete- | | |
| MouseNum7858 | | Hotspot- | | | Codon 61 | Chr5: 1295250 | rious | | |
| | | Mutant | | | | G > A | | | |
| WM4237- | PDX | BRAF | BRAF | | | TERT | Delete- | | |
| 2MP2MouseNum0302 | | Hotspot- | V600E | | | Chr5: 1295250 | rious | | |
| | | Mutant | | | | G > A | | | |
| WM4237MP1 | PDX | BRAF | BRAF | | | TERT | Delete- | | |
| MouseNum8174 | | Hotspot- | V600E | | | Chr5: 1295250 | rious | | |
| | | Mutant | | | | G > A | | | |
| WM4242MP1 | PDX | Wildtype | | | | TERT | Delete- | | |
| MouseNum7647 | | | | | | Chr5: 1295228 | rious | | |
| | | | | | | G > A | | | |
| WM4243MP1 | PDX | BRAF | BRAF | | | TERT | Delete- | | |
| MouseNum7649 | | Hotspot- | V600E | | | Chr5: 1295228 | rious | | |
| | | Mutant | | | | G > A | | | |
| WM4248MP1 | PDX | BRAF | BRAF | | | TERT | Delete- | | |
| MouseNum8088 | | Hotspot- | V600E | | | Chr5: 1295250 | rious | | |
| | | Mutant | | | | G > A | | | |
| WM4250MP1 | PDX | Wildtype | | | | TERT | Delete- | | |
| MouseNum8072 | | | | | | Chr5: 1295228 | rious | | |
| | | | | | | G > A | | | |
| WM4251MP1 | PDX | RAS | | | NRAS | TERT | Delete- | | |
| MouseNum8074 | | Hotspot- | | | Codon 61 | Chr5: 1295228 | rious | | |
| | | Mutant | | | | G > A | | | |
| WM4254MP1 | PDX | Wildtype | | | | TERT | Delete- | | |
| MouseNum8794 | | | | | | Chr5: 1295228 | rious | | |
| | | | | | | G > A | | | |
| WM4255MP1 | PDX | BRAF | BRAF | | | TERT | Delete- | | |
| MouseNum8584 | | Hotspot- | V600K | | | Chr5: 1295228 | rious | | |
| | | Mutant | | | | G > A | | | |
| WM4257MP1 | PDX | NF1- | | | NRAS | TERT | Delete- | | |
| MouseNum8616 | | Mutant | | | Codon 61 | Chr5: 1295228 | rious | | |
| | | | | | | G > A | | | |
| WM4260MP1 | PDX | KIT- | | | | TERT | Delete- | | |
| MouseNum8641 | | Mutant | | | | Chr5: 1295250 | rious | | |
| | | | | | | G > A | | | |
| WM4262CPLXMP2 | PDX | RAS | BRAF | | NRAS | TERT | Delete- | | |
| MouseNum8592 | | Hotspot- | V600E | | 12/3 | Chr5: 1295228 | rious | | |
| | | Mutant | | | | G > A | | | |
| WM4264- | PDX | BRAF | BRAF | | | TERT | Delete- | | |
| 2MP1MouseNum9997 | | Hotspot- | V600E | | | Chr5: 1295228 | rious | | |
| | | Mutant | | | | G > A | | | |
| WM4266MP1 | PDX | Wildtype | | | | TERT | Delete- | | |
| MouseNum9157 | | | | | | Chr5: 1295250 | rious | | |
| | | | | | | G > A | | | |
| WM4274MP1 | PDX | Wildtype | | | | TERT | Delete- | | |
| MouseNum8588 | | | | | | Chr5: 1295228 | rious | | |
| | | | | | | G > A | | | |
| WM4276MP1 | PDX | BRAF | BRAF | | | TERT | Delete- | | |
| MouseNum8599 | | Hotspot- | V600E | | | Chr5: 1295228 | rious | | |
| | | Mutant | | | | G > A | | | |
| WM4280PLXMP1 | PDX | RAS | BRAF | | NRAS | TERT | Delete- | | |
| MouseNum9005 | | Hotspot- | V600K | | 12/3 | Chr5: 1295228 | rious | | |
| | | Mutant | | | | G > A | | | |
| WM4285MP1 | PDX | BRAF | BRAF | | | TERT | Delete- | | |
| MouseNum9571 | | Hotspot- | V600E | | | Chr5: 1295250 | rious | | |
| | | Mutant | | | | G > A | | | |
| WM4286- | PDX | BRAF | BRAF | | | TERT | Delete- | | |
| 1MP1MouseNum9741 | | Hotspot- | V600E | | | Chr5: 1295250 | rious | | |
| | | Mutant | | | | G > A | | | |
| WM4292MP1 | PDX | BRAF | BRAF | | | TERT | Delete- | | |
| MouseNum9914 | | Hotspot- | V600E | | | Chr5: 1295250 | rious | | |
| | | Mutant | | | | G > A | | | |
| WM4295- | PDX | Wildtype | | | | TERT | Delete- | | |
| 2MP1MouseNum0041 | | | | | | Chr5: 1295250 | rious | | |
| | | | | | | G > A | | | |
| WM4295- | PDX | Wildtype | | | | TERT | Delete- | | |
| 3MP1MouseNum0045 | | | | | | Chr5: 1295250 | rious | | |
| | | | | | | G > A | | | |
| WM4295MP1 | PDX | Wildtype | | | | TERT | Delete- | | |
| MouseNum9772 | | | | | | Chr5: 1295250 | rious | | |
| | | | | | | G > A | | | |
| WM4299-1MP1 | PDX | RAS | | | NRAS | TERT | Delete- | | |
| | | Hotspot- | | | Codon 61 | Chr5: 1295228 | rious | | |
| | | Mutant | | | | G > A | | | |
| WM4299- | PDX | RAS | | | NRAS | TERT | Delete- | | |
| 2MP1MouseNum9947 | | Hotspot- | | | Codon 61 | Chr5: 1295228 | rious | | |
| | | Mutant | | | | G > A | | | |
| WM4299- | PDX | RAS | | | NRAS | TERT | Delete- | | |
| 3MP1MouseNum9988 | | Hotspot- | | | Codon 61 | Chr5: 1295228 | rious | | |
| | | Mutant | | | | G > A | | | |
| WM4299- | PDX | RAS | | | NRAS | TERT | Delete- | | |
| 4MP1MouseNum9994 | | Hotspot- | | | Codon 61 | Chr5: 1295228 | rious | | |
| | | Mutant | | | | G > A | | | |
| WM4319MP1 | PDX | RAS | | | NRAS | TERT | Delete- | | |
| MouseNum0381 | | Hotspot- | | | Codon 61 | Chr5: 1295228 | rious | | |
| | | Mutant | | | | G > A | | | |
| WM4324MP1 | PDX | BRAF | BRAF | | | TERT | Delete- | | |
| MouseNum0753 | | Hotspot- | V600E | | | Chr5: 1295250 | rious | | |
| | | Mutant | | | | G > A | | | |
| WM4330MP1 | PDX | Wildtype | Non- | | | TERT | Delete- | | |
| MouseNum0831 | | | BRAF | | | Chr5: 1295228 | rious | | |
| | | | Hotspot | | | G > A | | | |
| WTH202MP4 | PDX | Wildtype | | | | TERT | Delete- | | |
| MouseNum8340 | | | | | | Chr5: 1295228 | rious | | |
| | | | | | | G > A | | | |
| WM3926MP2 | PDX | BRAF | BRAF | | | TERT | Delete- | TERT | VUS |
| MouseNum6234 | | Hotspot- | V600E | | | Chr5: 1295250 | rious | chr5: 1295205 | |
| | | Mutant | | | | G > A | | G > A | |
| WM3926PLXMP4 | PDX | BRAF | BRAF | | | TERT | Delete- | TERT | VUS |
| MouseNum8063 | | Hotspot- | V600E | | | Chr5: 1295250 | rious | chr5: 1295205 | |
| | | Mutant | | | | G > A | | G > A | |
| WM3977MP2 | PDX | BRAF | BRAF | | | TERT | Delete- | TERT | VUS |
| MouseNum3383 | | Hotspot- | V600K | | | Chr5: 1295228 | rious | chr5: 1295205 | |
| | | Mutant | | | | G > A | | G > A | |
| WM4053- | PDX | BRAF | BRAF | | | TERT | Delete- | TERT | VUS |
| 1MP1MouseNum5202 | | Hotspot- | V600E | | | Chr5: 1295228 | rious | Chr5: 1295490 | |
| | | Mutant | | | | G > A | | C > A | |
| WM4068MP1 | PDX | RAS | Non- | Codon | | TERT | Delete- | TERT | VUS |
| MouseNum6517 | | Hotspot- | BRAF | 12/13 | | Chr5: 1295228- | rious | chr5: 1295230 | |
| | | Mutant | Hotspot | | | 1295229 | | G > A | |
| | | | | | | GG > AA | | | |
| WM4099MP1 | PDX | NF1- | | | | TERT | Delete- | TERT | VUS |
| MouseNum6233 | | Mutant | | | | Chr5: 1295242- | rious | chr5: 1295200 | |
| | | | | | | 1295243 | | G > A | |
| | | | | | | GG > AA | | | |
| WM4214MP1 | PDX | RAS | | | NRAS | TERT | Delete- | TERT | VUS |
| MouseNum6835 | | Hotspot- | | | Codon 61 | Chr5: 1295228 | rious | chr5: 1295230 | |
| | | Mutant | | | | G > A | | G > A | |
| WM4281MP1 | PDX | NF1- | | | | TERT | Delete- | TERT | VUS |
| MouseNum9139 | | Mutant | | | | Chr5: 1295228 | rious | chr5: 1295230 | |
| | | | | | | G > A | | G > A | |
| WM4351- | PDX | BRAF | BRAF | | | TERT | Delete- | TERT | VUS |
| 2MP1MouseNum1154 | | Hotspot- | V600E | | | Chr5: 1295228 | rious | chr5: 1295230 | |
| | | Mutant | | | | G > A | | G > A | |
| WM3902MP5 | PDX | BRAF | BRAF | | | TERT | Delete- | TERT | Delete- |
| MouseNum6556 | | Hotspot- | V600E | | | Chr5: 1295228 | rious | Chr5: 1295250 | rious |
| | | Mutant | | | | G > A | | G > A | |
| WM3934MP1 | PDX | BRAF | BRAF | | | TERT | Delete- | TERT | Delete- |
| MouseNum1757 | | Hotspot- | V600E | | | Chr5: 1295250 | rious | Chr5: 1295250 | rious |
| | | Mutant | | | | G > A | | G > A | |
| WM3907- | PDX | BRAF | BRAF | | | No TERT | No TERT | No TERT | No TERT |
| 1MP3MouseNum3414 | | Hotspot- | V600E | | | Promoter | Promoter | Promoter | Promoter |
| | | Mutant | | | | Mutation | Mutation | Mutation | Mutation |
| WM3909MP6 | PDX | BRAF | BRAF | | | No TERT | No TERT | No TERT | No TERT |
| MouseNum6870 | | Hotspot- | V600E | | | Promoter | Promoter | Promoter | Promoter |
| | | Mutant | | | | Mutation | Mutation | Mutation | Mutation |
| WM3940MP1 | PDX | BRAF | BRAF | | | No TERT | No TERT | No TERT | No TERT |
| MouseNum1716 | | Hotspot- | V600E | | | Promoter | Promoter | Promoter | Promoter |
| | | Mutant | | | | Mutation | Mutation | Mutation | Mutation |
| WM3940MP4 | PDX | BRAF | BRAF | | | No TERT | No TERT | No TERT | No TERT |
| MouseNum4860 | | Hotspot- | V600E | | | Promoter | Promoter | Promoter | Promoter |
| | | Mutant | | | | Mutation | Mutation | Mutation | Mutation |
| WM3960CPLXMP4 | PDX | BRAF | BRAF | | | No TERT | No TERT | No TERT | No TERT |
| MouseNum5838 | | Hotspot- | V600E | | | Promoter | Promoter | Promoter | Promoter |
| | | Mutant | | | | Mutation | Mutation | Mutation | Mutation |
| WM3963- | PDX | BRAF | BRAF | | | No TERT | No TERT | No TERT | No TERT |
| 2MP3MouseNum8176 | | Hotspot- | V600E | | | Promoter | Promoter | Promoter | Promoter |
| | | Mutant | | | | Mutation | Mutation | Mutation | Mutation |
| WM3965- | PDX | BRAF | BRAF | | | No TERT | No TERT | No TERT | No TERT |
| 2MP1MouseNum3054 | | Hotspot- | V600E | | | Promoter | Promoter | Promoter | Promoter |
| | | Mutant | | | | Mutation | Mutation | Mutation | Mutation |
| WM3971MP3 | PDX | BRAF | BRAF | | | No TERT | No TERT | No TERT | No TERT |
| MouseNum3395 | | Hotspot- | V600E | | | Promoter | Promoter | Promoter | Promoter |
| | | Mutant | | | | Mutation | Mutation | Mutation | Mutation |
| WM4002MP1 | PDX | BRAF | BRAF | | | No TERT | No TERT | No TERT | No TERT |
| MouseNum5243 | | Hotspot- | V600E | | | Promoter | Promoter | Promoter | Promoter |
| | | Mutant | | | | Mutation | Mutation | Mutation | Mutation |
| WM4011MP2 | PDX | BRAF | BRAF | | | No TERT | No TERT | No TERT | No TERT |
| MouseNum5833 | | Hotspot- | V600K | | | Promoter | Promoter | Promoter | Promoter |
| | | Mutant | | | | Mutation | Mutation | Mutation | Mutation |
| WM4052MP2 | PDX | BRAF | BRAF | | | No TERT | No TERT | No TERT | No TERT |
| MouseNum7685 | | Hotspot- | V600E | | | Promoter | Promoter | Promoter | Promoter |
| | | Mutant | | | | Mutation | Mutation | Mutation | Mutation |
| WM4061MP2 | PDX | BRAF | BRAF | | | No TERT | No TERT | No TERT | No TERT |
| MouseNum6593 | | Hotspot- | V600E | | | Promoter | Promoter | Promoter | Promoter |
| | | Mutant | | | | Mutation | Mutation | Mutation | Mutation |
| WM4070- | PDX | BRAF | BRAF | | | No TERT | No TERT | No TERT | No TERT |
| 1MP2MouseNum6546 | | Hotspot- | V600K | | | Promoter | Promoter | Promoter | Promoter |
| | | Mutant | | | | Mutation | Mutation | Mutation | Mutation |
| WM4096MP1 | PDX | BRAF | BRAF | | | No TERT | No TERT | No TERT | No TERT |
| MouseNum6666 | | Hotspot- | V600E | | | Promoter | Promoter | Promoter | Promoter |
| | | Mutant | | | | Mutation | Mutation | Mutation | Mutation |
| WM4125MP3 | PDX | BRAF | BRAF | | | No TERT | No TERT | No TERT | No TERT |
| MouseNum1330 | | Hotspot- | V600E | | | Promoter | Promoter | Promoter | Promoter |
| | | Mutant | | | | Mutation | Mutation | Mutation | Mutation |
| WM4191MP2 | PDX | BRAF | BRAF | | | No TERT | No TERT | No TERT | No TERT |
| MouseNum9869 | | Hotspot- | V600E | | | Promoter | Promoter | Promoter | Promoter |
| | | Mutant | | | | Mutation | Mutation | Mutation | Mutation |
| WM4198MP3 | PDX | BRAF | BRAF | | | No TERT | No TERT | No TERT | No TERT |
| MouseNum19821984 | | Hotspot- | V600E | | | Promoter | Promoter | Promoter | Promoter |
| | | Mutant | | | | Mutation | Mutation | Mutation | Mutation |
| WM4200MP1 | PDX | BRAF | BRAF | | | No TERT | No TERT | No TERT | No TERT |
| MouseNum6669 | | Hotspot- | V600E | | | Promoter | Promoter | Promoter | Promoter |
| | | Mutant | | | | Mutation | Mutation | Mutation | Mutation |
| WM4249MP1 | PDX | BRAF | BRAF | | Likely | No TERT | No TERT | No TERT | No TERT |
| MouseNum8724 | | Hotspot- | V600E | | Delete- | Promoter | Promoter | Promoter | Promoter |
| | | Mutant | | | rious | Mutation | Mutation | Mutation | Mutation |
| | | | | | Missense | | | | |
| WM4298MP1 | PDX | BRAF | BRAF | | | No TERT | No TERT | No TERT | No TERT |
| MouseNum9018 | | Hotspot- | V600E | | | Promoter | Promoter | Promoter | Promoter |
| | | Mutant | | | | Mutation | Mutation | Mutation | Mutation |
| WM3936- | PDX | RAS | BRAF | | NRAS | No TERT | No TERT | No TERT | No TERT |
| 2MP1MouseNum2975RECUT | | Hotspot- | V600E | | Codon 61 | Promoter | Promoter | Promoter | Promoter |
| | | Mutant | | | | Mutation | Mutation | Mutation | Mutation |
| WM3936- | PDX | RAS | BRAF | | NRAS | No TERT | No TERT | No TERT | No TERT |
| 2MP5MouseNum3646 | | Hotspot- | V600E | | Codon 61 | Promoter | Promoter | Promoter | Promoter |
| | | Mutant | | | | Mutation | Mutation | Mutation | Mutation |
| WM3972MP2 | PDX | RAS | | | NRAS | No TERT | No TERT | No TERT | No TERT |
| MouseNum3241RECUT | | Hotspot- | | | Codon 61 | Promoter | Promoter | Promoter | Promoter |
| | | Mutant | | | | Mutation | Mutation | Mutation | Mutation |
| WM3974MP3 | PDX | RAS | | | NRAS | No TERT | No TERT | No TERT | No TERT |
| MouseNum2726 | | Hotspot- | | | 12/3 | Promoter | Promoter | Promoter | Promoter |
| | | Mutant | | | | Mutation | Mutation | Mutation | Mutation |
| WM3974MP4 | PDX | RAS | | | NRAS | No TERT | No TERT | No TERT | No TERT |
| MouseNum8186 | | Hotspot- | | | 12/3 | Promoter | Promoter | Promoter | Promoter |
| | | Mutant | | | | Mutation | Mutation | Mutation | Mutation |
| WM4067MP1 | PDX | RAS | | Codon | | No TERT | No TERT | No TERT | No TERT |
| MouseNum6254 | | Hotspot- | | 12/13 | | Promoter | Promoter | Promoter | Promoter |
| | | Mutant | | | | Mutation | Mutation | Mutation | Mutation |
| WM4180MP1 | PDX | RAS | BRAF | | NRAS | No TERT | No TERT | No TERT | No TERT |
| | | Hotspot- | V600E | | Codon 61 | Promoter | Promoter | Promoter | Promoter |
| | | Mutant | | | | Mutation | Mutation | Mutation | Mutation |
| WM4331MP1 | PDX | RAS | | | NRAS | No TERT | No TERT | No TERT | No TERT |
| MouseNum0552 | | Hotspot- | | | Codon 61 | Promoter | Promoter | Promoter | Promoter |
| | | Mutant | | | | Mutation | Mutation | Mutation | Mutation |
| WM4233MP1 | PDX | NF1- | | | NRAS | No TERT | No TERT | No TERT | No TERT |
| MouseNum7641 | | Mutant | | | Codon 61 | Promoter | Promoter | Promoter | Promoter |
| | | | | | | Mutation | Mutation | Mutation | Mutation |
| WM4175MP3 | PDX | KIT- | | | | No TERT | No TERT | No TERT | No TERT |
| MouseNum9065 | | Mutant | | | | Promoter | Promoter | Promoter | Promoter |
| | | | | | | Mutation | Mutation | Mutation | Mutation |
| WM4279MP1 | PDX | KIT- | | | | No TERT | No TERT | No TERT | No TERT |
| MouseNum9791 | | Mutant | | | | Promoter | Promoter | Promoter | Promoter |
| | | | | | | Mutation | Mutation | Mutation | Mutation |
| BM550MP2 | PDX | Wildtype | | | | No TERT | No TERT | No TERT | No TERT |
| MouseNum7161 | | | | | | Promoter | Promoter | Promoter | Promoter |
| | | | | | | Mutation | Mutation | Mutation | Mutation |
| WM4010MP1 | PDX | Wildtype | | | | No TERT | No TERT | No TERT | No TERT |
| MouseNum4551 | | | | | | Promoter | Promoter | Promoter | Promoter |
| | | | | | | Mutation | Mutation | Mutation | Mutation |
| WM4034MP1 | PDX | Wildtype | | | | No TERT | No TERT | No TERT | No TERT |
| MouseNum4587 | | | | | | Promoter | Promoter | Promoter | Promoter |
| | | | | | | Mutation | Mutation | Mutation | Mutation |
| WM4039- | PDX | Wildtype | | | | No TERT | No TERT | No TERT | No TERT |
| 2MP1MouseNum5538 | | | | | | Promoter | Promoter | Promoter | Promoter |
| | | | | | | Mutation | Mutation | Mutation | Mutation |
| WM4058MP1 | PDX | Wildtype | | | | No TERT | No TERT | No TERT | No TERT |
| MouseNum5536 | | | | | | Promoter | Promoter | Promoter | Promoter |
| | | | | | | Mutation | Mutation | Mutation | Mutation |
| WM4087LiMP1 | PDX | Wildtype | | | | No TERT | No TERT | No TERT | No TERT |
| MouseNum6265 | | | | | | Promoter | Promoter | Promoter | Promoter |
| | | | | | | Mutation | Mutation | Mutation | Mutation |
| WM4173MP1 | PDX | Wildtype | | | | No TERT | No TERT | No TERT | No TERT |
| MouseNum8174 | | | | | | Promoter | Promoter | Promoter | Promoter |
| | | | | | | Mutation | Mutation | Mutation | Mutation |
| WM4175MP4 | PDX | Wildtype | | | | No TERT | No TERT | No TERT | No TERT |
| MouseNum8522 | | | | | | Promoter | Promoter | Promoter | Promoter |
| | | | | | | Mutation | Mutation | Mutation | Mutation |
| WM4188MP3 | PDX | Wildtype | | | | No TERT | No TERT | No TERT | No TERT |
| MouseNum9625 | | | | | | Promoter | Promoter | Promoter | Promoter |
| | | | | | | Mutation | Mutation | Mutation | Mutation |
| WM4216MP1 | PDX | Wildtype | | | | No TERT | No TERT | No TERT | No TERT |
| MouseNum7349 | | | | | | Promoter | Promoter | Promoter | Promoter |
| | | | | | | Mutation | Mutation | Mutation | Mutation |
| WM4222MP1 | PDX | Wildtype | | | | No TERT | No TERT | No TERT | No TERT |
| MouseNum7427 | | | | | | Promoter | Promoter | Promoter | Promoter |
| | | | | | | Mutation | Mutation | Mutation | Mutation |
| WM4226MP1 | PDX | Wildtype | | | | No TERT | No TERT | No TERT | No TERT |
| MouseNum7426 | | | | | | Promoter | Promoter | Promoter | Promoter |
| | | | | | | Mutation | Mutation | Mutation | Mutation |
| WM4240MP1 | PDX | Wildtype | | | | No TERT | No TERT | No TERT | No TERT |
| MouseNum8721 | | | | | | Promoter | Promoter | Promoter | Promoter |
| | | | | | | Mutation | Mutation | Mutation | Mutation |
| WM4263MP1 | PDX | Wildtype | | | | No TERT | No TERT | No TERT | No TERT |
| MouseNum8859 | | | | | | Promoter | Promoter | Promoter | Promoter |
| | | | | | | Mutation | Mutation | Mutation | Mutation |
| WM4307- | PDX | Wildtype | | | | No TERT | No TERT | No TERT | No TERT |
| 2MP1MouseNum9655 | | | | | | Promoter | Promoter | Promoter | Promoter |
| | | | | | | Mutation | Mutation | Mutation | Mutation |
| WM4309MP1 | PDX | Wildtype | | | | No TERT | No TERT | No TERT | No TERT |
| MouseNum0161 | | | | | | Promoter | Promoter | Promoter | Promoter |
| | | | | | | Mutation | Mutation | Mutation | Mutation |
| WM4323MP1 | PDX | Wildtype | | | | No TERT | No TERT | No TERT | No TERT |
| MouseNum0757 | | | | | | Promoter | Promoter | Promoter | Promoter |
| | | | | | | Mutation | Mutation | Mutation | Mutation |
| WM4341MP1 | PDX | Wildtype | | | | No TERT | No TERT | No TERT | No TERT |
| MouseNum1040 | | | | | | Promoter | Promoter | Promoter | Promoter |
| | | | | | | Mutation | Mutation | Mutation | Mutation |
| PM171MP1Mouse | PDX | BRAF | BRAF | | | No TERT | No TERT | No TERT | No TERT |
| Num8860RECUT | | Hotspot- | V600E | | | Promoter | Promoter | Promoter | Promoter |
| | | Mutant | | | | Mutation | Mutation | Mutation | Mutation |
| WM3901PLXMP3 | PDX | BRAF | BRAF | | | No TERT | No TERT | No TERT | No TERT |
| MouseNum6529 | | Hotspot- | V600E | | | Promoter | Promoter | Promoter | Promoter |
| | | Mutant | | | | Mutation | Mutation | Mutation | Mutation |
| WM3929MP5 | PDX | BRAF | BRAF | | | TERT | Delete- | | |
| MouseNum5364 | | Hotspot- | V600E | | | Chr5: 1295250 | rious | | |
| | | Mutant | | | | G > A | | | |
| WM3929MP6PLX | PDX | BRAF | BRAF | | | TERT | Delete- | | |
| MouseNum6566RECUT | | Hotspot- | V600E | | | Chr5: 1295250 | rious | | |
| | | Mutant | | | | G > A | | | |
| WM3935MP1 | PDX | BRAF | BRAF | | | TERT | Delete- | | |
| MouseNum1752 | | Hotspot- | V600E | | | Chr5: 1295250 | rious | | |
| | | Mutant | | | | G > A | | | |
| WM3964MP1 | PDX | RAS | Non- | Codon 61 | | No TERT | No TERT | No TERT | No TERT |
| MouseNum5198 | | Hotspot- | BRAF | | | Promoter | Promoter | Promoter | Promoter |
| | | Mutant | Hotspot | | | Mutation | Mutation | Mutation | Mutation |
| WM3981MP1 | PDX | BRAF | BRAF | | | TERT | Delete- | | |
| | | Hotspot- | V600E | | | Chr5: 1295250 | rious | | |
| | | Mutant | | | | G > A | | | |
| WM3992MP2 | PDX | RAS | | | NRAS | TERT | Delete- | | |
| MouseNum4501 | | Hotspot- | | | Codon 61 | Chr5: 1295228 | rious | | |
| | | Mutant | | | | G > A | | | |
| WM3993MP1 | PDX | BRAF | BRAF | | | TERT | Delete- | | |
| MouseNum5183 | | Hotspot- | V600E | | | Chr5: 1295228 | rious | | |
| | | Mutant | | | | G > A | | | |
| WM4007MP1 | PDX | BRAF | BRAF | | | No TERT | No TERT | No TERT | No TERT |
| MouseNum8970 | | Hotspot- | V600E | | | Promoter | Promoter | Promoter | Promoter |
| | | Mutant | | | | Mutation | Mutation | Mutation | Mutation |
| WM4077MP3 | PDX | NF1- | Non- | | NRAS | No TERT | No TERT | No TERT | No TERT |
| MouseNum7109 | | Mutant | BRAF | | 12/3 | Promoter | Promoter | Promoter | Promoter |
| | | | Hotspot | | | Mutation | Mutation | Mutation | Mutation |
| WM4082MP1 | PDX | NF1- | | | | No TERT | No TERT | No TERT | No TERT |
| MouseNum6215 | | Mutant | | | | Promoter | Promoter | Promoter | Promoter |
| | | | | | | Mutation | Mutation | Mutation | Mutation |
| WM4088MP1 | PDX | BRAF | BRAF | | | TERT | VUS | | |
| MouseNum5845 | | Hotspot- | V600E | | | Chr5: 1295242 | | | |
| | | Mutant | | | | G > A | | | |
| WM4211MP1 | PDX | BRAF | BRAF | | | TERT | Delete- | | |
| MouseNum6779 | | Hotspot- | V600E | | | Chr5: 1295228 | rious | | |
| | | Mutant | | | | G > A | | | |
| WM4228MP1 | PDX | BRAF | Other | | | TERT | Delete- | TERT | VUS |
| MouseNum7653 | | Hotspot- | BRAF | | | Chr5: 1295228 | rious | Chr5: 1295230 | |
| | | Mutant | Hotspot | | | G > A | | G > A | |
| WM4239MP1 | PDX | BRAF | BRAF | | | TERT | Delete- | | |
| MouseNum8082 | | Hotspot- | V600K | | | Chr5: 1295250 | rious | | |
| | | Mutant | | | | G > A | | | |
| WM4247MP1 | PDX | BRAF | BRAF | | | TERT | Delete- | | |
| MouseNum8087 | | Hotspot- | V600K | | | Chr5: 1295250 | rious | | |
| | | Mutant | | | | G > A | | | |
| WM4265- | PDX | RAS | | | NRAS | TERT | Delete- | TERT | VUS |
| 1MP1MouseNum8532 | | Hotspot- | | | Codon 61 | Chr5: 1295250 | rious | chr5: 1295205 | |
| | | Mutant | | | | G > A | | G > A | |
| WM4265- | PDX | RAS | | | NRAS | TERT | Delete- | TERT | VUS |
| 2MP1MouseNum8534 | | Hotspot- | | | Codon 61 | Chr5: 1295250 | rious | chr5: 1295205 | |
| | | Mutant | | | | G > A | | G > A | |
| WM4267MP2 | PDX | Wildtype | | | | No TERT | No TERT | No TERT | No TERT |
| MouseNum8578 | | | | | | Promoter | Promoter | Promoter | Promoter |
| | | | | | | Mutation | Mutation | Mutation | Mutation |
| WM4272MP1 | PDX | RAS | | | NRAS | TERT | Delete- | TERT | VUS |
| MouseNum8251 | | Hotspot- | | | Codon 61 | Chr5: 1295250 | rious | chr5: 1295205 | |
| | | Mutant | | | | G > A | | G > A | |
| WM4273MP1 | PDX | RAS | | | NRAS | TERT | Delete- | | |
| MouseNum8852 | | Hotspot- | | | Codon 61 | Chr5: 1295228 | rious | | |
| | | Mutant | | | | G > A | | | |
| WM4311MP1 | PDX | BRAF | BRAF | | | TERT | Delete- | | |
| MouseNum9937 | | Hotspot- | V600E | | | Chr5: 1295228 | rious | | |
| | | Mutant | | | | G > A | | | |
| WM4314MP1 | PDX | NF1- | | | | TERT | Delete- | | |
| MouseNum0008 | | Mutant | | | | Chr5: 1295250 | rious | | |
| | | | | | | G > A | | | |

TABLE 4
TERT promoter mutations in Wistar's treatment-resistand PDX

| | | Com- | | | Variant | |
|---|---|---|---|---|---|---|
| Sample | Variant | ments | Call | Gene | Type | Zyg |
| WM3901PLXMP3 | BRAF:NM_004333:exon15:c.T1799A:p.V600E | | Delete- | BRAF | nonsynony- | het |
| MouseNum6529 | | | rious | | mous SNV | |
| WM3901PLXMP3 | CDKN2A:NM_000077:exon2:c.197_219del:p.66_73del | | Delete- | CDKN2A | frameshift | het |
| MouseNum6529 | | | rious | | deletion | |
| WM3901PLXMP3 | FLT3:NM_004119:exon8:c.C956T:p.S319L | | Likely | FLT3 | nonsynony- | het |
| MouseNum6529 | | | Delete- | | mous SNV | |
| | | | rious | | | |
| WM3901PLXMP3 | KIT:NM_000222:exon2:c.G152A:p.G51D | | Likely | KIT | nonsynony- | het |
| MouseNum6529 | | | Delete- | | mous SNV | |
| | | | rious | | | |
| WM3901PLXMP3 | PIK3CA:NM_006218:exon16:c.T2368C:p.S790P | | VUS | PIK3CA | nonsynony- | het |
| MouseNum6529 | | | | | mous SNV | |
| WM3901PLXMP4p7 | BRAF:NM_004333:exon15:c.T1799A:p.V600E | | Delete- | BRAF | nonsynony- | het |
| | | | rious | | mous SNV | |
| WM3901PLXMP4p7 | CDKN2A:NM_000077:exon2:c.197_219del: | | Delete- | CDKN2A | nonsynony- | het |
| | p.66_73del | | rious | | mous SNV | |
| WM3901PLXMP4p7 | FLT3:NM_004119:exon8:c.C956T:p.S319L | | Likely | FLT3 | nonsynony- | het |
| | | | Delete- | | mous SNV | |
| | | | rious | | | |
| WM3901PLXMP4p7 | KIT:NM_000222:exon2:c.G152A:p.G51D | | Likely | KIT | nonsynony- | het |
| | | | Delete- | | mous SNV | |
| | | | rious | | | |
| WM3901PLXMP4p7 | PIK3CA:NM_006218:exon16:c.T2368C:p.S790P | | VUS | PIK3CA | nonsynony- | het |
| | | | | | mous SNV | |
| WM3901PLXMP4p7 | TERT Chr5: 1295250 G > A | | Delete- | TERT | Promoter | het |
| | | | rious | | SNV | |
| WM3903- | BRAF:NM_004333:exon15:c.T1799A:p.V600E | | Delete- | BRAF | nonsynony- | het |
| 1MP5MouseNum4045 | | | rious | | mous SNV | |
| WM3903- | ERBB2:NM_004448:exon10:c.G1185C:p.E395D | | VUS | ERBB2 | nonsynony- | het |
| 1MP5MouseNum4045 | | | | | mous SNV | |
| WM3903- | MAP2K1:NM_002755:exon2:c.A167C:p.Q56P | | Delete- | MAP2K1 | nonsynony- | het |
| 1MP5MouseNum4045 | | | rious | | mous SNV | |
| WM3903- | MAP2K1:NM_002755:exon2:c.G182A:p.G61E | | VUS | MAP2K1 | nonsynony- | het |
| 1MP5MouseNum4045 | | | | | mous SNV | |
| WM3903- | PREX2:NM_024870:exon11:c.G1261A:p.E421K | | Likely | PREX2 | nonsynony- | het |
| 1MP5MouseNum4045 | | | Delete- | | mous SNV | |
| | | | rious | | | |
| WM3903- | PREX2:NM_024870:exon29:c.C3577G:p.P1193A | | VUS | PREX2 | nonsynony- | het |
| 1MP5MouseNum4045 | | | | | mous SNV | |
| WM3903- | TERT Chr5: 1295242 G > A | | VUS | TERT | Promoter | het |
| 1MP5MouseNum4045 | | | | | SNV | |
| WM3903- | TRRAP:NM_001244580:exon18:c.C2165T:p.S722F | | VUS | TRRAP | nonsynony- | het |
| 1MP5MouseNum4045 | | | | | mous SNV | |
| WM3903- | VAV3:NM_006113:exon25:c.C2294T:p.P765L | | Likely | VAV3 | nonsynony- | het |
| 1MP5MouseNum4045 | | | Delete- | | mous SNV | |
| | | | rious | | | |
| WM3903- | APC:NM_001127511:exon14:c.G2380A:p.D794N | | VUS | APC | nonsynony- | het |
| 1MP6MouseNum6576 | | | | | mous SNV | |
| WM3903- | ARID1A:NM_006015:exon2:c.C1174T:p.P392S | | VUS | ARID1A | nonsynony- | het |
| 1MP6MouseNum6576 | | | | | mous SNV | |
| WM3903- | ATM:NM_000051:exon10:c.C1270T:p.P424S | | Likely | ATM | nonsynony- | het |
| 1MP6MouseNum6576 | | | Delete- | | mous SNV | |
| | | | rious | | | |
| WM3903- | ATM:NM_000051:exon14:c.G2198A:p.G733D | | VUS | ATM | nonsynony- | het |
| 1MP6MouseNum6576 | | | | | mous SNV | |
| WM3903- | ATM:NM_000051:exon59:c.C8602T:p.L2868F | | Likely | ATM | nonsynony- | het |
| 1MP6MouseNum6576 | | | Delete- | | mous SNV | |
| | | | rious | | | |
| WM3903- | BRAF:NM_004333:exon15:c.T1799A:p.V600E | | Delete- | BRAF | nonsynony- | het |
| 1MP6MouseNum6576 | | | rious | | mous SNV | |
| WM3903- | CDKN2B:NM_004936:exon2:c.G338A:p.G113D | | Likely | CDKN2B | nonsynony- | het |
| 1MP6MouseNum6576 | | | Delete- | | mous SNV | |
| | | | rious | | | |
| WM3903- | DCC:NM_005215:exon18:c.G2786A:p.S929N | | Likely | DCC | nonsynony- | het |
| 1MP6MouseNum6576 | | | Delete- | | mous SNV | |
| | | | rious | | | |
| WM3903- | ERBB3:NM_001982:exon12:c.G1355A:p.G452E | | Likely | ERBB3 | nonsynony- | het |
| 1MP6MouseNum6576 | | | Delete- | | mous SNV | |
| | | | rious | | | |
| WM3903- | ERBB4:NM_005235:exon13:c.G1540A:p.G514R | | Likely | ERBB4 | nonsynony- | het |
| 1MP6MouseNum6576 | | | Delete- | | mous SNV | |
| | | | rious | | | |
| WM3903- | FBXW7:NM_033632:exon4:c.G593A:p.G198E | | VUS | FBXW7 | nonsynony- | het |
| 1MP6MouseNum6576 | | | | | mous SNV | |
| WM3903- | FLT3:NM_004119:exon6:c.G718A:p.E240K | | VUS | FLT3 | nonsynony- | het |
| 1MP6MouseNum6576 | | | | | mous SNV | |
| WM3903- | GNA11:NM_002067:exon5:c.G727A:p.D243N | | Likely | GNA11 | nonsynony- | het |
| 1MP6MouseNum6576 | | | Delete- | | mous SNV | |
| | | | rious | | | |
| WM3903- | GRM3:NM_000840:exon5:c.T2531A:p.F844Y | | VUS | GRM3 | nonsynony- | het |
| 1MP6MouseNum6576 | | | | | mous SNV | |
| WM3903- | IDH1:NM_005896:exon4:c.C152T:p.A51V | | Likely | IDH1 | nonsynony- | het |
| 1MP6MouseNum6576 | | | Delete- | | mous SNV | |
| | | | rious | | | |
| WM3903- | KDR:NM_002253:exon15:c.G2245A:p.E749K | | Likely | KDR | nonsynony- | het |
| 1MP6MouseNum6576 | | | Delete- | | mous SNV | |
| | | | rious | | | |
| WM3903- | KDR:NM_002253:exon16:c.2353_2354del:p.I785fs | | Delete- | KDR | frameshift | het |
| 1MP6MouseNum6576 | | | rious | | deletion | |
| WM3903- | MAP2K1:NM_002755:exon2:c.A167C:p.Q56P | | Delete- | MAP2K1 | nonsynony- | het |
| 1MP6MouseNum6576 | | | rious | | mous SNV | |
| WM3903- | MDM4:NM_002393:exon5:c.C289T:p.P97S | | Likely | MDM4 | nonsynony- | het |
| 1MP6MouseNum6576 | | | Delete- | | mous SNV | |
| | | | rious | | | |
| WM3903- | MITF:NM_198159:exon6:c.C848T:p.A283V | | VUS | MITF | nonsynony- | het |
| 1MP6MouseNum6576 | | | | | mous SNV | |
| WM3903- | NOTCH1:NM_017617:exon10:c.C1606T:p.P536S | | Likely | NOTCH1 | nonsynony- | het |
| 1MP6MouseNum6576 | | | Delete- | | mous SNV | |
| | | | rious | | | |
| WM3903- | NOTCH1:NM_017617:exon18:c.G2807A:p.G936D | | Likely | NOTCH1 | nonsynony- | het |
| 1MP6MouseNum6576 | | | Delete- | | mous SNV | |
| | | | rious | | | |
| WM3903- | NOTCH1:NM_017617:exon22:c.G3587A:p.G1196D | | Likely | NOTCH1 | nonsynony- | het |
| 1MP6MouseNum6576 | | | Delete- | | mous SNV | |
| | | | rious | | | |
| WM3903- | PREX2:NM_024870:exon5:c.C506T:p.P169L | | Likely | PREX2 | nonsynony- | het |
| 1MP6MouseNum6576 | | | Delete- | | mous SNV | |
| | | | rious | | | |
| WM3903- | SMO:NM_005631:exon10:c.C1678T:p.P560S | | Likely | SMO | nonsynony- | het |
| 1MP6MouseNum6576 | | | Delete- | | mous SNV | |
| | | | rious | | | |
| WM3903- | TACC1:NM_006283:exon5:c.C1472T:p.S491F | | VUS | TACC1 | nonsynony- | het |
| 1MP6MouseNum6576 | | | | | mous SNV | |
| WM3903- | TERT Chr5: 1295228 G > A | | Delete- | TERT | Promoter | hom_alt |
| 1MP6MouseNum6576 | | | rious | | SNV | |
| WM3903- | VAV3:NM_006113:exon10:c.G964A:p.D322N | | Likely | VAV3 | nonsynony- | het |
| 1MP6MouseNum6576 | | | Delete- | | mous SNV | |
| | | | rious | | | |
| WM3903- | APC:NM_001127511:exon14:c.G2380A:p.D794N | | VUS | APC | nonsynony- | het |
| 1MP6PLXp15 | | | | | mous SNV | |
| WM3903- | ARID1A:NM_006015:exon2:c.C1174T:p.P392S | | VUS | ARID1A | nonsynony- | het |
| 1MP6PLXp15 | | | | | mous SNV | |
| WM3903- | ATM:NM_000051:exon10:c.C1270T:p.P424S | | VUS | ATM | nonsynony- | het |
| 1MP6PLXp15 | | | | | mous SNV | |
| WM3903- | ATM:NM_000051:exon14:c.G2198A:p.G733D | | VUS | ATM | nonsynony- | het |
| 1MP6PLXp15 | | | | | mous SNV | |
| WM3903- | ATM:NM_000051:exon59:c.C8602T:p.L2868F | | Likely | ATM | nonsynony- | het |
| 1MP6PLXp15 | | | Delete- | | mous SNV | |
| | | | rious | | | |
| WM3903- | BAP1:NM_004656:exon17:c.G2123A:p.R708Q | | VUS | BAP1 | nonsynony- | het |
| 1MP6PLXp15 | | | | | mous SNV | |
| WM3903- | BRAF:NM_004333:exon15:c.T1799A:p.V600E | | Delete- | BRAF | nonsynony- | het |
| 1MP6PLXp15 | | | rious | | mous SNV | |
| WM3903- | CDKN2B:NM_004936:exon2:c.G338A:p.G113D | | Likely | CDKN2B | nonsynony- | hom |
| 1MP6PLXp15 | | | Delete- | | mous SNV | |
| | | | rious | | | |
| WM3903- | DCC:NM_005215:exon18:c.G2786A:p.S929N | | Likely | DCC | nonsynony- | het |
| 1MP6PLXp15 | | | Delete- | | mous SNV | |
| | | | rious | | | |
| WM3903- | ERBB2:NM_004448:exon14:c.C1648T:p.L550F | | VUS | ERBB2 | nonsynony- | het |
| 1MP6PLXp15 | | | | | mous SNV | |
| WM3903- | ERBB3:NM_001982:exon12:c.G1355A:p.G452E | | Likely | ERBB3 | nonsynony- | het |
| 1MP6PLXp15 | | | Delete- | | mous SNV | |
| | | | rious | | | |
| WM3903- | ERBB4:NM_005235:exon13:c.G1540A:p.G514R | | Likely | ERBB4 | nonsynony- | het |
| 1MP6PLXp15 | | | Delete- | | mous SNV | |
| | | | rious | | | |
| WM3903- | FBXW7:NM_033632:exon4:c.G593A:p.G198E | | VUS | FBXW7 | nonsynony- | het |
| 1MP6PLXp15 | | | | | mous SNV | |
| WM3903- | GRM3:NM_000840:exon5:c.T2531A:p.F844Y | | VUS | GRM3 | nonsynony- | het |
| 1MP6PLXp15 | | | | | mous SNV | |
| WM3903- | IDH1:NM_005896:exon4:c.C152T:p.A51V | | Likely | IDH1 | nonsynony- | het |
| 1MP6PLXp15 | | | Delete- | | mous SNV | |
| | | | rious | | | |
| WM3903- | KDR:NM_002253:exon15:c.G2245A:p.E749K | | Likely | KDR | nonsynony- | het |
| 1MP6PLXp15 | | | Delete- | | mous SNV | |
| | | | rious | | | |
| WM3903- | KDR:NM_002253:exon16:c.2353_2354del:p.785_785del | | Delete- | KDR | frameshift | het |
| 1MP6PLXp15 | | | rious | | deletion | |
| WM3903- | MAP2K1(NM_002755:exon5:c.517 − 1G > A, | | Likely | MAP2K1 | splicing | het |
| 1MP6PLXp15 | p.V173_splice | | Delete- | | | |
| | | | rious | | | |
| WM3903- | MAP2K1:NM_002755:exon2:c.A167C:p.Q56P | | Delete- | MAP2K1 | nonsynony- | het |
| 1MP6PLXp15 | | | rious | | mous SNV | |
| WM3903- | MAP3K5:NM_005923:exon17:c.G2369A:p.G790E | | Likely | MAP3K5 | nonsynony- | hom |
| 1MP6PLXp15 | | | Delete- | | mous SNV | |
| | | | rious | | | |
| WM3903- | MDM4:NM_002393:exon5:c.C289T:p.P97S | | Likely | MDM4 | nonsynony- | het |
| 1MP6PLXp15 | | | Delete- | | mous SNV | |
| | | | rious | | | |
| WM3903- | MITF:NM_198159:exon6:c.C848T:p.A283V | | VUS | MITF | nonsynony- | het |
| 1MP6PLXp15 | | | | | mous SNV | |
| WM3903- | NF1:NM_001042492:exon17:c.1877delT:p.L626fs | | Delete- | NF1 | frameshift | het |
| 1MP6PLXp15 | | | rious | | deletion | |
| WM3903- | NOTCH1:NM_017617:exon10:c.C1606T:p.P536S | | Likely | NOTCH1 | nonsynony- | het |
| 1MP6PLXp 15 | | | Delete- | | mous SNV | |
| | | | rious | | | |
| WM3903- | NOTCH1:NM_017617:exon18:c.G2807A:p.G936D | | Likely | NOTCH1 | nonsynony- | het |
| 1MP6PLXp15 | | | Delete- | | mous SNV | |
| | | | rious | | | |
| WM3903- | NOTCH1:NM_017617:exon22:c.G3587A:p.G1196D | | Likely | NOTCH1 | nonsynony- | het |
| 1MP6PLXp15 | | | Delete- | | mous SNV | |
| | | | rious | | | |
| WM3903- | PREX2:NM_024870:exon5:c.C506T:p.P169L | | Likely | PREX2 | nonsynony- | het |
| 1MP6PLXp15 | | | Delete- | | mous SNV | |
| | | | rious | | | |
| WM3903- | SMO:NM_005631:exon10:c.C1678T:p.P560S | | Likely | SMO | nonsynony- | het |
| 1MP6PLXp15 | | | Delete- | | mous SNV | |
| | | | rious | | | |
| WM3903- | TACC1:NM_006283:exon5:c.C1472T:p.S491F | | VUS | TACC1 | nonsynony- | het |
| 1MP6PLXp15 | | | | | mous SNV | |
| WM3903- | TERT Chr5: 1295228 G > A | | Delete- | TERT | Promoter | hom |
| 1MP6PLXp15 | | | rious | | SNV | |
| WM3903- | VAV3:NM_006113:exon10:c.G964A:p.D322N | | Likely | VAV3 | nonsynony- | het |
| 1MP6PLXp15 | | | Delete- | | mous SNV | |
| | | | rious | | | |
| WM3907- | BRAF:NM_004333:exon15:c.T1799A:p.V600E | | Delete- | BRAF | nonsynony- | hom |
| 1MP3MouseNum3414 | | | rious | | mous SNV | |
| WM3907- | GRIN2A:NM_001134407:exon3:c.G536A:p.G179D | | Likely | GRIN2A | nonsynony- | het |
| 1MP3MouseNum3414 | | | Delete- | | mous SNV | |
| | | | rious | | | |
| WM3907- | TRRAP:NM_001244580:exon27:c.C3871T:p.P1291S | | Likely | TRRAP | nonsynony- | het |
| 1MP3MouseNum3414 | | | Delete- | | mous SNV | |
| | | | rious | | | |
| WM3908MP3Mouse | ARID1A:NM_006015:exon20:c.G6770A:p.R2257Q | | Likely | ARID1A | nonsynony- | het |
| Num1563RECUT | | | Delete- | | mous SNV | |
| | | | rious | | | |
| WM3908MP3Mouse | BRAF:NM_004333:exon15:c.GT1798AA:p.V600K | | Delete- | BRAF | nonsynony- | het |
| Num1563RECUT | | | rious | | mous SNV | |
| WM3908MP3Mouse | CTNNB1:NM_001904:exon3:c.C134T:p.S45F | | Delete- | CTNNBl | nonsynony- | het |
| Num1563RECUT | | | rious | | mous SNV | |
| WM3908MP3Mouse | FBXW7:NM_033632:exon2:c.349_351del:p.117_117del | | Likely | FBXW7 | nonsynony- | het |
| Num1563RECUT | | | Delete- | | mous SNV | |
| | | | rious | | | |
| WM3908MP3Mouse | GRM3:NM_000840:exon2:c.C65T:p.S22F | | VUS | GRM3 | nonsynony- | het |
| Num1563RECUT | | | | | mous SNV | |
| WM3908MP3Mouse | IDH1:NM_005896:exon4:c.C394T:p.R132C | | Delete- | IDH1 | nonsynony- | het |
| Num1563RECUT | | | rious | | mous SNV | |
| WM3908MP3Mouse | SMARCA4:NM_001128849:exon6:c.C895T:p.P299S | | VUS | SMARCA4 | nonsynony- | het |
| Num1563RECUT | | | | | mous SNV | |
| WM3908MP3Mouse | TERT Chr5: 1295250 G > A | | Delete- | TERT | Promoter | het |
| Num1563RECUT | | | rious | | SNV | |
| WM3908MP3Mouse | VAV3:NM_006113:exon19:c.G1736A:p.R579Q | | VUS | VAV3 | nonsynony- | het |
| Num1563RECUT | | | | | mous SNV | |
| WM3908MP4PLXMouse | ARID1A:NM_006015:exon20:c.G6770A:p.R2257Q | | Likely | ARID1A | nonsynony- | het |
| Num6433RECUT | | | Delete- | | mous SNV | |
| | | | rious | | | |
| WM3908MP4PLXMouse | BRAF:NM_004333:exon15:c.GT1798AA:p.V600K | | Delete- | BRAF | nonsynony- | het |
| Num6433RECUT | | | rious | | mous SNV | |
| WM3908MP4PLXMouse | CTNNB1:NM_001904:exon3:c.C134T:p.S45F | | Delete- | CTNNB1 | nonsynony- | het |
| Num6433RECUT | | | rious | | mous SNV | |
| WM3908MP4PLXMouse | DDX3X:NM_001356:exon15:c.A1694C:p.Q565P | | VUS | DDX3X | nonsynony- | het |
| Num6433RECUT | | | | | mous SNV | |
| WM3908MP4PLXMouse | FBXW7:NM_033632:exon2:c.349_351del:p.117_117del | | Likely | FBXW7 | nonsynony- | het |
| Num6433RECUT | | | Delete- | | mous SNV | |
| | | | rious | | | |
| WM3908MP4PLXMouse | GRM3:NM_000840:exon2:c.C65T:p.S22F | | VUS | GRM3 | nonsynony- | het |
| Num6433RECUT | | | | | mous SNV | |
| WM3908MP4PLXMouse | IDH1:NM_005896:exon4:c.C394T:p.R132C | | Delete- | IDH1 | nonsynony- | het |
| Num6433RECUT | | | rious | | mous SNV | |
| WM3908MP4PLXMouse | SMARCA4:NM_001128849:exon6:c.C895T:p.P299S | | VUS | SMARCA4 | nonsynony- | het |
| Num6433RECUT | | | | | mous SNV | |
| WM3908MP4PLXMouse | TERT Chr5: 1295250 G > A | | Delete- | TERT | Promoter | het |
| Num6433RECUT | | | rious | | SNV | |
| WM3908MP4PLXMouse | VAV3:NM_006113:exon19:c.G1736A:p.R579Q | | VUS | VAV3 | nonsynony- | het |
| Num6433RECUT | | | | | mous SNV | |
| WM3909MP6Mouse | BRAF:NM_004333:exon15:c.T1799A:p.V600E | | Delete- | BRAF | nonsynony- | het |
| Num6870 | | | rious | | mous SNV | |
| WM3909MP6Mouse | ROS1:NM_002944:exon30:c.C5035T:p.P1679S | | VUS | ROS1 | nonsynony- | het |
| Num6870 | | | | | mous SNV | |
| WM3909MP6Mouse | ROS1:NM_002944:exon6:c.C551T:p.P184L | | VUS | ROS1 | nonsynony- | het |
| Num6870 | | | | | mous SNV | |
| WM3909MP6Mouse | SMARCA4:NM_001128849:exon34:c.C4859T:p.S1620F | | VUS | SMARCA4 | nonsynony- | het |
| Num6870 | | | | | mous SNV | |
| WM3909PLX2MP7 | BRAF:NM_004333:exon15:c.T1799A:p.V600E | | Delete- | BRAF | nonsynony- | het |
| | | | rious | | mous SNV | |
| WM3909PLX2MP7 | ROS1:NM_002944:exon30:c.C5035T:p.P1679S | | VUS | ROS1 | nonsynony- | het |
| | | | | | mous SNV | |
| WM3909PLX2MP7 | ROS1:NM_002944:exon6:c.C551T:p.P184L | | VUS | ROS1 | nonsynony- | het |
| | | | | | mous SNV | |
| WM3909PLX2MP7 | SMARCA4:NM_001128849:exon34:c.C4859T:p.S1620F | | VUS | SMARCA4 | nonsynony- | het |
| | | | | | mous SNV | |
| WM3909PLX2MP7 | TERT Chr5: 1295250 G > A | | Delete- | TERT | Promoter | het |
| | | | rious | | SNV | |
| WM3926MP2Mouse | BRAF:NM_004333:exon15:c.T1799A:p.V600E | | Delete- | BRAF | nonsynony- | het |
| Num6234 | | | rious | | mous SNV | |
| WM3926MP2Mouse | CDK4:NM_000075:exon2:c.C70T:p.R24C | Germline | Delete- | CDK4 | nonsynony- | het |
| Num6234 | | Suscepti- | rious | | mous SNV | |
| | | bility | | | | |
| | | Mutation | | | | |
| WM3926MP2Mouse | FLT3:NM_004119:exon3:c.C305T:p.S102F | | VUS | FLT3 | nonsynony- | hom |
| Num6234 | | | | | mous SNV | |
| WM3926MP2Mouse | PTEN(NM_000314:exon1:c.79 + 1G > A), | | Likely | PTEN | splicing | hom |
| Num6234 | p.Y27_splice | | Delete- | | | |
| | | | rious | | | |
| WM3926MP2Mouse | PTPRD:NM_002839:exon33:c.G3875T:p.R1292M | | VUS | PTPRD | nonsynony- | hom |
| Num6234 | | | | | mous SNV | |
| WM3926MP2Mouse | TERT chr5: 1295205 G > A | | VUS | TERT | Promoter | het |
| Num6234 | | | | | SNV | |
| WM3926MP2Mouse | TERT Chr5: 1295250 G > A | | Delete- | TERT | Promoter | het |
| Num6234 | | | rious | | SNV | |
| WM3926MP3p5Mouse | BRAF:NM_004333:exon15:c.T1799A:p.V600E | | Delete- | BRAF | nonsynony- | het |
| Num7864 | | | rious | | mous SNV | |
| WM3926MP3p5Mouse | CDK4:NM_000075:exon2:c.C70T:p.R24C | Germline | Delete- | CDK4 | nonsynony- | het |
| Num7864 | | Suscepti- | rious | | mous SNV | |
| | | bility | | | | |
| | | Mutation | | | | |
| WM3926MP3p5Mouse | FLT3:NM_004119:exon3:c.C305T:p.S102F | | VUS | FLT3 | nonsynony- | hom_alt |
| Num7864 | | | | | mous SNV | |
| WM3926MP3p5Mouse | PTEN:NM_000314:exon1:c.79 + 1G > A | | Likely | PTEN | splicing | hom_alt |
| Num7864 | | | Delete- | | | |
| | | | rious | | | |
| WM3926MP3p5Mouse | PTPRD:NM_002839:exon33:c.G3875T:p.R1292M | | VUS | PTPRD | nonsynony- | hom_alt |
| Num7864 | | | | | mous SNV | |
| WM3926MP3p5Mouse | TERT chr5: 1295205 G > A | | VUS | TERT | Promoter | het |
| Num7864 | | | | | SNV | |
| WM3926MP3p5Mouse | TERT Chr5: 1295250 G > A | | Delete- | TERT | Promoter | het |
| Num7864 | | | rious | | SNV | |
| WM3926PLXMP4Mouse | BRAF:NM_004333:exon15:c.T1799A:p.V600E | | Delete- | BRAF | nonsynony- | het |
| Num8063 | | | rious | | mous SNV | |
| WM3926PLXMP4Mouse | CDK4:NM_000075:exon2:c.C70T:p.R24C | Germline | Delete- | CDK4 | nonsynony- | het |
| Num8063 | | Suscepti- | rious | | mous SNV | |
| | | bility | | | | |
| | | Mutation | | | | |
| WM3926PLXMP4Mouse | FLT3:NM_004119:exon3:c.C305T:p.S102F | | VUS | FLT3 | nonsynony- | hom |
| Num8063 | | | | | mous SNV | |
| WM3926PLXMP4Mouse | PTEN(NM_000314:exon1:c.79 + 1G > A), | | Likely | PTEN | splicing | hom |
| Num8063 | p.Y27_splice | | Delete- | | | |
| | | | rious | | | |
| WM3926PLXMP4Mouse | PTPRD:NM_002839:exon33:c.G3875T:p.R1292M | | VUS | PTPRD | nonsynony- | hom |
| Num8063 | | | | | mous SNV | |
| WM3926PLXMP4Mouse | TERT chr5: 1295205 G > A | | VUS | TERT | Promoter | het |
| Num8063 | | | | | SNV | |
| WM3926PLXMP4Mouse | TERT Chr5: 1295250 G > A | | Delete- | TERT | Promoter | het |
| Num8063 | | | rious | | SNV | |
| WM3926PLXMP4PLXp10 | BRAF:NM_004333:exon15:c.T1799A:p.V600E | | Delete- | BRAF | nonsynony- | het |
| | | | rious | | mous SNV | |
| WM3926PLXMP4PLXp10 | CDK4:NM_000075:exon2:c.C70T:p.R24C | | Likely | CDK4 | nonsynony- | het |
| | | | Delete- | | mous SNV | |
| | | | rious | | | |
| WM3926PLXMP4PLXp10 | FLT3:NM_004119:exon3:c.C305T:p.S102F | | VUS | FLT3 | nonsynony- | hom_alt |
| | | | | | mous SNV | |
| WM3926PLXMP4PLXp10 | MAP2K1:NM_002755:exon3:c.G362C:p.C121S | | Likely | MAP2K1 | nonsynony- | het |
| | | | Delete- | | mous SNV | |
| | | | rious | | | |
| WM3926PLXMP4PLXp10 | PTEN:NM_001304717:exon2:c.598 + 1G > A | | Likely | PTEN | splicing | hom_alt |
| | | | Delete- | | | |
| | | | rious | | | |
| WM3926PLXMP4PLXp10 | PTPRD:NM_002839:exon33:c.G3875T:p.R1292M | | VUS | PTPR1D | nonsynony- | hom_alt |
| | | | | | mous SNV | |
| WM3926PLXMP4PLXp10 | TERT chr5:1295205 G > A | | VUS | TERT | Promoter | hom_alt |
| | | | | | SNV | |
| WM3926PLXMP4PLXp10 | TERT Chr5:1295250 G > A | | Delete- | TERT | Promoter | hom_alt |
| | | | rious | | SNV | |
| WM3929MCPLXMP7p6 | ALK:NM_004304:exon15:c.G2579C:p.R860T | | Likely | ALK | nonsynony- | het |
| MouseNum6538 | | | Delete- | | mous SNV | |
| | | | rious | | | |
| WM3929MCPLXMP7p6 | BRAF:NM_004333:exon13:c.C1624T:p.H542Y | | Delete- | BRAF | nonsynony- | het |
| MouseNum6538 | | | rious | | mous SNV | |
| WM3929MCPLXMP7p6 | BRAF:NM_004333:exon15:c.T1799A:p.V600E | | Delete- | BRAF | nonsynony- | het |
| MouseNum6538 | | | rious | | mous SNV | |
| WM3929MCPLXMP7p6 | CDKN2A:NM_000077:exon2:c.C341T:p.P114L | | Delete- | CDKN2A | nonsynony- | hom |
| MouseNum6538 | | | rious | | mous SNV | |
| WM3929MCPLXMP7p6 | EZH1:NM_001991:exon12:c.C1336T:p.H446Y | | VUS | EZH1 | nonsynony- | het |
| MouseNum6538 | | | | | mous SNV | |
| WM3929MCPLXMP7p6 | FLT3:NM_004119:exon3:c.G296A:p.G99E | | VUS | FLT3 | nonsynony- | het |
| MouseNum6538 | | | | | mous SNV | |
| WM3929MCPLXMP7p6 | JAK3:NM_000215:exon8:c.C1094T:p.P365L | | VUS | JAK3 | nonsynony- | hom |
| MouseNum6538 | | | | | mous SNV | |
| WM3929MCPLXMP7p6 | PTPRD:NM_002839:exon29:c.C3073T:p.H1025Y | | VUS | PTPRD | nonsynony- | hom |
| MouseNum6538 | | | | | mous SNV | |
| WM3929MCPLXMP7p6 | SOX10:NM_006941:exon2:c.A191C:p.D64A | | VUS | SOX10 | nonsynony- | het |
| MouseNum6538 | | | | | mous SNV | |
| WM3929MCPLXMP7p6 | TERT Chr5: 1295250 G > A | | Delete- | TERT | Promoter | het |
| MouseNum6538 | | | rious | | SNV | |
| WM3929MCPLXMP7p6 | TP53:NM_000546:exon5:c.T428G:p.V143G | | Delete- | TP53 | nonsynony- | horn |
| MouseNum6538 | | | rious | | mous SNV | |
| WM3929PLXMP5PLXp15 | ALK:NM_004304:exon15:c.G2579C:p.R860T | | Likely | ALK | nonsynony- | het |
| | | | Delete- | | mous SNV | |
| | | | rious | | | |
| WM3929PLXMP5PLXp15 | BRAF:NM_004333:exon13:c.C1624T:p.H542Y | | Likely | BRAF | nonsynony- | het |
| | | | Delete- | | mous SNV | |
| | | | rious | | | |
| WM3929PLXMP5PLXp15 | BRAF:NM_004333:exon15:c.T1799A:p.V600E | | Delete- | BRAF | nonsynony- | het |
| | | | rious | | mous SNV | |
| WM3929PLXMP5PLXp15 | CDKN2A:NM_000077:exon2:c.C341T:p.P114L | | Delete- | CDKN2A | nonsynony- | hom_alt |
| | | | rious | | mous SNV | |
| WM3929PLXMP5PLXp15 | CTNNB1:NM_001904:exon3:c.123_149del:p.41_50del | | Likely | CTNNB1 | nonsynony- | het |
| | | | Delete- | | mous SNV | |
| | | | rious | | | |
| WM3929PLXMP5PLXp15 | EZH1:NM_001991:exon12:c.C1336T:p.H446Y | | VUS | EZH1 | nonsynony- | het |
| | | | | | mous SNV | |
| WM3929PLXMP5PLXp15 | FLT3:NM_004119:exon3:c.G296A:p.G99E | | VUS | FLT3 | nonsynony- | het |
| | | | | | mous SNV | |
| WM3929PLXMP5PLXp15 | JAK3:NM_000215:exon8:c.C1094T:p.P365L | | Likely | JAK3 | nonsynony- | hom_alt |
| | | | Delete- | | mous SNV | |
| | | | rious | | | |
| WM3929PLXMP5PLXp15 | PTPRD:NM_002839:exon29:c.C3073T:p.H1025Y | | VUS | PTPRD | nonsynony- | hom_alt |
| | | | | | mous SNV | |
| WM3929PLXMP5PLXp15 | SOX10:NM_006941:exon2:c.A191C:p.D64A | | Likely | SOX10 | nonsynony- | het |
| | | | Delete- | | mous SNV | |
| | | | rious | | | |
| WM3929PLXMP5PLXp15 | TERT Chr5: 1295250 G > A | | Delete- | TERT | Promoter | het |
| | | | rious | | SNV | |
| WM3929PLXMP5PLXp15 | TP53:NM_000546:exon5:c.T428G:p.V143G | | Delete- | TP53 | nonsynony- | hom_alt |
| | | | rious | | mous SNV | |
| WM3935MP1Mouse | BRAF:NM_004333:exon15:c.T1799A:p.V600E | | Delete- | BRAF | nonsynony- | het |
| Num1752 | | | rious | | mous SNV | |
| WM3935MP1Mouse | CDKN2A:NM_000077:exon2:c.T389A:p.L130Q | | Likely | CDKN2A | nonsynony- | hom_alt |
| Num1752 | | | Delete- | | mous SNV | |
| | | | rious | | | |
| WM3935MP1Mouse | CTNNB1:NM_001904:exon9:c.G1358A:p.R453Q | | VUS | CTNNB1 | nonsynony- | het |
| Num1752 | | | | | mous SNV | |
| WM3935MP1Mouse | KDR:NM_002253:exon21:c.G2824A:p.G942R | | Likely | KDR | nonsynony- | het |
| Num1752 | | | Delete- | | mous SNV | |
| | | | rious | | | |
| WM3935MP1Mouse | MAP3K5:NM_005923:exon30:c.G4093A:p.A1365T | | Likely | MAP3K5 | nonsynony- | hom_alt |
| Num1752 | | | Delete- | | mous SNV | |
| | | | rious | | | |
| WM3935MP1Mouse | NF2:NM_000268:exon14:c.C1448T:p.P483L | | VUS | NF2 | nonsynony- | het |
| Num1752 | | | | | mous SNV | |
| WM3935MP1Mouse | SMARCA4:NM_001128849:exon29:c.G3976A:p.E1326K | | Likely | SMARCA4 | nonsynony- | het |
| Num1752 | | | Delete- | | mous SNV | |
| | | | rious | | | |
| WM3935MP1Mouse | TERT Chr5: 1295250 G > A | | Delete- | TERT | Promoter | het |
| Num1752 | | | rious | | SNV | |
| WM3935MP1Mouse | TERT:NM_198253:exon5:c.G2006A:p.R669Q | | Likely | TERT | nonsynony- | het |
| Num1752 | | | Delete- | | mous SNV | |
| | | | rious | | | |
| WM3935MP1Mouse | TP53:NM_000546:exon5:c.G542A:p.R181H | | Delete- | TP53 | nonsynony- | hom_alt |
| Num1752 | | | rious | | mous SNV | |
| WM3936- | BRAF:NM_004333:exon15:c.T1799A:p.V600E | | Delete- | BRAF | nonsynony- | het |
| 1MP3PLXp4Mouse | | | rious | | mous SNV | |
| Num3850 | | | | | | |
| WM3936- | NRAS:NM_002524:exon3:c.C181A:p.Q61K | | Delete- | NRAS | nonsynony- | het |
| 1MP3PLXp4Mouse | | | rious | | mous SNV | |
| Num3850 | | | | | | |
| WM3936- | PIK3CA:NM_006218:exon21:c.C3139T:p.H1047Y | | Delete- | PIK3CA | nonsynony- | het |
| 1MP3PLXp4Mouse | | | rious | | mous SNV | |
| Num3850 | | | | | | |
| WM3936- | PTEN:NM_000314:exon5:c.G314A:p.C105Y | | Likely | PTEN | nonsynony- | hom |
| 1MP3PLXp4Mouse | | | Delete- | | mous SNV | |
| Num3850 | | | rious | | | |
| WM3936- | TERT Chr5: 1295250 G > A | | Delete- | TERT | Promoter | het |
| 1MP3PLXp4Mouse | | | rious | | SNV | |
| Num3850 | | | | | | |
| WM3936- | BRAF:NM_004333:exon15:c.T1799A:p.V600E | | Delete- | BRAF | nonsynony- | het |
| 2MP1MouseNum2975 | | | rious | | mous SNV | |
| RECUT | | | | | | |
| WM3936- | NRAS:NM_002524:exon3:c.C181A:p.Q61K | | Delete- | NRAS | nonsynony- | het |
| 2MP1MouseNum2975 | | | rious | | mous SNV | |
| RECUT | | | | | | |
| WM3936- | PIK3CA:NM_006218:exon21:c.C3139T:p.H1047Y | | Delete- | PIK3CA | nonsynony- | het |
| 2MP1MouseNum2975 | | | rious | | mous SNV | |
| RECUT | | | | | | |
| WM3936- | PTEN:NM_000314:exon5:c.G314A:p.C105Y | | Likely | PTEN | nonsynony- | het |
| 2MP1MouseNum2975 | | | Delete- | | mous SNV | |
| RECUT | | | rious | | | |
| WM3936- | BRAF:NM_004333:exon15:c.T1799A:p.V600E | | Delete- | BRAF | nonsynony- | het |
| 2MP5MouseNum3646 | | | rious | | mous SNV | |
| WM3936- | NRAS:NM_002524:exon3:c.C181A:p.Q61K | | Delete- | NRAS | nonsynony- | het |
| 2MP5MouseNum3646 | | | rious | | mous SNV | |
| WM3936- | PIK3CA:NM_006218:exon21:c.C3139T:p.H1047Y | | Delete- | PIK3CA | nonsynony- | het |
| 2MP5MouseNum3646 | | | rious | | mous SNV | |
| WM3936- | PTEN:NM_000314:exon5:c.G314A:p.C105Y | | Likely | PTEN | nonsynony- | hom |
| 2MP5MouseNum3646 | | | Delete- | | mous SNV | |
| | | | rious | | | |
| WM3939- | BRAF:NM_004333:exon15:c.T1799A:p.V600E | | Delete- | BRAF | nonsynony- | het |
| 2CPLXMP5p7Mouse | | | rious | | mous SNV | |
| Num5183 | | | | | | |
| WM3939- | ERBB2:NM_004448:exon10:c.G1185C:p.E395D | | VUS | ERBB2 | nonsynony- | het |
| 2CPLXMP5p7Mouse | | | | | mous SNV | |
| Num5183 | | | | | | |
| WM3939- | MAP2K1:NM_002755:exon2:c.A167C:p.Q56P | | Delete- | MAP2K1 | nonsynony- | het |
| 2CPLXMP5p7Mouse | | | rious | | mous SNV | |
| Num5183 | | | | | | |
| WM3939- | MAP2K1:NM_002755:exon2:c.G182A:p.G61E | | VUS | MAP2K1 | nonsynony- | het |
| 2CPLXMP5p7Mouse | | | | | mous SNV | |
| Num5183 | | | | | | |
| WM3939- | MAP2K2:NM_030662:exon3:c.G426C:p.E142D | | Likely | MAP2K2 | nonsynony- | het |
| 2CPLXMP5p7Mouse | | | Delete- | | mous SNV | |
| Num5183 | | | rious | | | |
| WM3939- | PREX2:NM_024870:exon11:c.G1261A:p.E421K | | Likely | PREX2 | nonsynony- | het |
| 2CPLXMP5p7Mouse | | | Delete- | | mous SNV | |
| Num5183 | | | rious | | | |
| WM3939- | PREX2:NM_024870:exon29:c.C3577G:p.P1193A | | VUS | PREX2 | nonsynony- | het |
| 2CPLXMP5p7Mouse | | | | | mous SNV | |
| Num5183 | | | | | | |
| WM3939- | TERT Chr5: 1295242-1295243 GG > AA | | Delete- | TERT | Promoter | het |
| 2CPLXMP5p7Mouse | | | rious | | SNV | |
| Num5183 | | | | | | |
| WM3939- | TRRAP:NM_001244580:exon18:c.C2165T:p.S722F | | Likely | TRRAP | nonsynony- | het |
| 2CPLXMP5p7Mouse | | | Delete- | | mous SNV | |
| Num5183 | | | rious | | | |
| WM3939- | VAV3:NM_006113:exon25:c.C2294T:p.P765L | | Likely | VAV3 | nonsynony- | het |
| 2CPLXMP5p7Mouse | | | Delete- | | mous SNV | |
| Num5183 | | | rious | | | |
| WM3939- | BRAF:NM_004333:exon15:c.T1799A:p.V600E | | Delete- | BRAF | nonsynony- | het |
| 2CPLXMP6Mouse | | | rious | | mous SNV | |
| Num5900 | | | | | | |
| WM3939- | ERBB2:NM_004448:exon10:c.G1185C:p.E395D | | VUS | ERBB2 | nonsynony- | het |
| 2CPLXMP6Mouse | | | | | mous SNV | |
| Num5900 | | | | | | |
| WM3939- | MAP2K1:NM_002755:exon2:c.A167C:p.Q56P | | Delete- | MAP2K1 | nonsynony- | het |
| 2CPLXMP6Mouse | | | rious | | mous SNV | |
| Num5900 | | | | | | |
| WM3939- | MAP2K1:NM_002755:exon2:c.G182A:p.G61E | | VUS | MAP2K1 | nonsynony- | het |
| 2CPLXMP6Mouse | | | | | mous SNV | |
| Num5900 | | | | | | |
| WM3939- | PREX2:NM_024870:exon11:c.G1261A:p.E421K | | Likely | PREX2 | nonsynony- | het |
| 2CPLXMP6Mouse | | | Delete- | | mous SNV | |
| Num5900 | | | rious | | | |
| WM3939- | PREX2:NM_024870:exon29:c.C3577G:p.P1193A | | VUS | PREX2 | nonsynony- | het |
| 2CPLXMP6Mouse | | | | | mous SNV | |
| Num5900 | | | | | | |
| WM3939- | TERT Chr5: 1295242 G > A | | VUS | TERT | Promoter | het |
| 2CPLXMP6Mouse | | | | | SNV | |
| Num5900 | | | | | | |
| WM3939- | TRRAP:NM_001244580:exon18:c.C2165T:p.S722F | | VUS | TRRAP | nonsynony- | het |
| 2CPLXMP6Mouse | | | | | mous SNV | |
| Num5900 | | | | | | |
| WM3939- | VAV3:NM_006113:exon25:c.C2294T:p.P765L | | Likely | VAV3 | nonsynony- | het |
| 2CPLXMP6Mouse | | | Delete- | | mous SNV | |
| Num5900 | | | rious | | | |
| WM3942CPLXMP3Mouse | ABL1:NM_005157:exon6:c.G1015C:p.V339L | | Likely | ABL1 | nonsynony- | het |
| Num8885 | | | Delete- | | mous SNV | |
| | | | rious | | | |
| WM3942CPLXMP3Mouse | BRAF:NM_004333:exon15:c.T1799A:p.V600E | | Delete- | BRAF | nonsynony- | het |
| Num8885 | | | rious | | mous SNV | |
| WM3942CPLXMP3Mouse | MAP2K2:NM_030662:exon3:c.T373A:p.C125S | | Delete- | MAP2K2 | nonsynony- | het |
| Num8885 | | | rious | | mous SNV | |
| WM3942CPLXMP3Mouse | PIK3CB:NM_006219:exon4:c.T694G:p.L232V | | VUS | PIK3CB | nonsynony- | het |
| Num8885 | | | | | mous SNV | |
| WM3942CPLXMP3Mouse | TERT Chr5: 1295228 G > A | | Delete- | TERT | Promoter | het |
| Num8885 | | | rious | | SNV | |
| WM3942MP3p5Mouse | ABL1:NM_005157:exon6:c.G1015C:p.V339L | | Likely | ABL1 | nonsynony- | het |
| Num8752 | | | Delete- | | mous SNV | |
| | | | rious | | | |
| WM3942MP3p5Mouse | BRAF:NM_004333:exon15:c.T1799A:p.V600E | | Delete- | BRAF | nonsynony- | het |
| Num8752 | | | rious | | mous SNV | |
| WM3942MP3p5Mouse | PIK3CB:NM_006219:exon4:c.T694G:p.L232V | | VUS | PIK3CB | nonsynony- | het |
| Num8752 | | | | | mous SNV | |
| WM3942MP3p5Mouse | TERT Chr5: 1295228 G > A | | Delete- | TERT | Promoter | het |
| Num8752 | | | rious | | SNV | |
| WM3960CPLXMP4Mouse | BRAF:NM_004333:exon15:c.T1799A:p.V600E | | Delete- | BRAF | nonsynony- | hom |
| Num5838 | | | rious | | mous SNV | |
| WM3960CPLXMP4Mouse | PTPRD:NM_002839:exon28:c.G3023A:p.S1008N | | VUS | PTPRD | nonsynony- | hom |
| Num5838 | | | | | mous SNV | |
| WM3960CPLXMP4p4 | BRAF:NM_004333:exon15:c.T1799A:p.V600E | | Delete- | BRAF | nonsynony- | hom_alt |
| | | | rious | | mous SNV | |
| WM3960CPLXMP4p4 | PTPRD:NM_002839:exon28:c.G3023A:p.S1008N | | VUS | PTPRD | nonsynony- | hom_alt |
| | | | | | mous SNV | |
| WM3960CPLXMP4p4 | TERT Chr5: 1295228 G > A | | Delete- | TERT | Promoter | hom_alt |
| | | | rious | | SNV | |
| WM3960MP2PLXp20 | BRAF:NM_004333:exon15:c.T1799A:p.V600E | | Delete- | BRAF | nonsynony- | hom_alt |
| MouseNum3448 | | | rious | | mous SNV | |
| WM3960MP2PLXp20 | NRAS:NM_002524:exon3:c.C181A:p.Q61K | | Delete- | NRAS | nonsynony- | het |
| MouseNum3448 | | | rious | | mous SNV | |
| WM3960MP2PLXp20 | PTPRD:NM_002839:exon28:c.G3023A:p.S1008N | | VUS | PTPRD | nonsynony- | hom_alt |
| MouseNum3448 | | | | | mous SNV | |
| WM3960MP2PLXp20 | TERT Chr5: 1295228 G > A | | Delete- | TERT | Promoter | hom_alt |
| MouseNum3448 | | | rious | | SNV | |
| WM3960MP4Mouse | BRAF:NM_004333:exon15:c.T1799A:p.V600E | | Delete- | BRAF | nonsynony- | hom |
| Num3960 | | | rious | | mous SNV | |
| WM3960MP4Mouse | PTPRD:NM_002839:exon28:c.G3023A:p.S1008N | | VUS | PTPRD | nonsynony- | horn |
| Num3960 | | | | | mous SNV | |
| WM3960MP4Mouse | TERT Chr5: 1295228 G > A | | Delete- | TERT | Promoter | hom |
| Num3960 | | | rious | | SNV | |
| WM3965- | BRAF:NM_004333:exon15:c.T1799A:p.V600E | | Delete- | BRAF | nonsynony- | het |
| 2MP1MouseNum3054 | | | rious | | mous SNV | |
| WM3973MP3Mouse | BRAF:NM_004333:exon15:c.T1799A:p.V600E | | Delete- | BRAF | nonsynony- | hom |
| Num3470 | | | rious | | mous SNV | |
| WM3973MP3Mouse | DDX3X:NM_001356:exon2:c.92_93insTA:p.S31fs | | Delete- | DDX3X | frameshift | hom |
| Num3470 | | | rious | | insertion | |
| WM3973MP3Mouse | MAP2K1:NM_002755:exon2:c.A169G:p.K57E | | Delete- | MAP2K1 | nonsynony- | het |
| Num3470 | | | rious | | mous SNV | |
| WM3973MP3Mouse | MAP2K1:NM_002755:exon6:c.G605A:p.G202E | | Likely | MAP2K1 | nonsynony- | het |
| Num3470 | | | Delete- | | mous SNV | |
| | | | rious | | | |
| WM3973MP3Mouse | RHOT1:NM_001033568:exon11:c.C865T:p.P289S | | Likely | RHOT1 | nonsynony- | het |
| Num3470 | | | Delete- | | mous SNV | |
| | | | rious | | | |
| WM3973MP3Mouse | TERT Chr5: 1295250 G > A | | Delete- | TERT | Promoter | het |
| Num3470 | | | rious | | SNV | |
| WM3973MP4Mouse | BRAF:NM_004333:exon15:c.T1799A:p.V600E | | Delete- | BRAF | nonsynony- | het |
| Num7144 | | | rious | | mous SNV | |
| WM3973MP4Mouse | DDX3X:NM_001356:exon2:c.92_93insTA:p.S31fs | | Delete- | DDX3X | frameshift | het |
| Num7144 | | | rious | | insertion | |
| WM3973MP4Mouse | MAP2K1:NM_002755:exon2:c.A169G:p.K57E | | Delete- | MAP2K1 | nonsynony- | het |
| Num7144 | | | rious | | mous SNV | |
| WM3973MP4Mouse | MAP2K1:NM_002755:exon6:c.G605A:p.G202E | | Likely | MAP2K1 | nonsynony- | het |
| Num7144 | | | Delete- | | mous SNV | |
| | | | rious | | | |
| WM3973MP4Mouse | RHOT1:NM_001033568:exon11:c.C865T:p.P289S | | Likely | RHOT1 | nonsynony- | het |
| Num7144 | | | Delete- | | mous SNV | |
| | | | rious | | | |
| WM3973MP4Mouse | TERT Chr5: 1295250 G > A | | Delete- | TERT | Promoter | het |
| Num7144 | | | rious | | SNV | |
| WM3973PLXMP5Mouse | BRAF:NM_004333:exon15:c.T1799A:p.V600E | | Delete- | BRAF | nonsynony- | het |
| Num7627 | | | rious | | mous SNV | |
| WM3973PLXMP5Mouse | DDX3X:NM_001356:exon2:c.92_93insTA:p.S31fs | | Delete- | DDX3X | frameshift | het |
| Num7627 | | | rious | | insertion | |
| WM3973PLXMP5Mouse | MAP2K1:NM_002755:exon2:c.A169G:p.K57E | | Delete- | MAP2K1 | nonsynony- | het |
| Num7627 | | | rious | | mous SNV | |
| WM3973PLXMP5Mouse | MAP2K1:NM_002755:exon6:c.G605A:p.G202E | | Likely | MAP2K1 | nonsynony- | het |
| Num7627 | | | Delete- | | mous SNV | |
| | | | rious | | | |
| WM3973PLXMP5Mouse | RHOT1:NM_001033568:exon1l:c.C865T:p.P289S | | Likely | RHOT1 | nonsynony- | het |
| Num7627 | | | Delete- | | mous SNV | |
| | | | rious | | | |
| WM3973PLXMP5Mouse | TERT Chr5: 1295250 G > A | | Delete- | TERT | Promoter | het |
| Num7627 | | | rious | | SNV | |
| WM3983PLXMP7Mouse | ARID1A:NM_006015:exon1:c.G707T:p.G236V | | VUS | ARID1A | nonsynony- | het |
| Num8003 | | | | | mous SNV | |
| WM3983PLXMP7Mouse | ATM:NM_000051:exon60:c.C8708T:p.P2903L | | Likely | ATM | nonsynony- | het |
| Num8003 | | | Delete- | | mous SNV | |
| | | | rious | | | |
| WM3983PLXMP7Mouse | BRAF:NM_004333:exon15:c.T1799A:p.V600K | | Delete- | BRAF | nonsynony- | het |
| Num8003 | | | rious | | mous SNV | |
| WM3983PLXMP7Mouse | EGFR:NM_005228:exon10:c.C1190T:p.T397I | | Likely | EGFR | nonsynony- | het |
| Num8003 | | | Delete- | | mous SNV | |
| | | | rious | | | |
| WM3983PLXMP7Mouse | EZH1:NM_001991:exon16:c.A1834G:p.K612E | | Likely | EZH1 | nonsynony- | het |
| Num8003 | | | Delete- | | mous SNV | |
| | | | rious | | | |
| WM3983PLXMP7Mouse | NRAS:NM_002524:exon3:c.C181A:p.Q61K | | Delete- | NRAS | nonsynony- | het |
| Num8003 | | | rious | | mous SNV | |
| WM3983PLXMP7Mouse | PPP6C:NM_001123355:exon8:c.C901T:p.R301C | | VUS | PPP6C | nonsynony- | het |
| Num8003 | | | | | mous SNV | |
| WM3983PLXMP7Mouse | PREX2:NM_024870:exon4:c.C349T:p.R117C | | VUS | PREX2 | nonsynony- | het |
| Num8003 | | | | | mous SNV | |
| WM3983PLXMP7Mouse | SETD2:NM_014159:exon3:c.C2164T:p.Q722X | | Delete- | SETD2 | stopgain | het |
| Num8003 | | | rious | | | |
| WM3983PLXMP7Mouse | TERT Chr5: 1295250 G > A | | Delete- | TERT | Promoter | hom |
| Num8003 | | | rious | | SNV | |
| WM3983PLXMP7Mouse | TRRAP:NM_001244580:exon62:c.C9584T:p.S3195L | | Likely | TRRAP | nonsynony- | het |
| Num8003 | | | Delete- | | mous SNV | |
| | | | rious | | | |
| WM4008MP2Mouse | ATM:NM_000051:exon47:c.G6912T:p.E2304D | | Likely | ATM | nonsynony- | het |
| Num4345 | | | Delete- | | mous SNV | |
| | | | rious | | | |
| WM4008MP2Mouse | BRAF:NM_004333:exon15:c.GT1798AA:p.V600K | | Delete- | BRAF | nonsynony- | het |
| Num4345 | | | rious | | mous SNV | |
| WM4008MP2Mouse | PREX2:NM_024870:exon6:c.G637A:p.E213K | | Likely | PREX2 | nonsynony- | het |
| Num4345 | | | Delete- | | mous SNV | |
| | | | rious | | | |
| WM4008MP2Mouse | RB1:NM_000321:exon25:c.A2635T:p.I879F | | Likely | RB1 | nonsynony- | het |
| Num4345 | | | Delete- | | mous SNV | |
| | | | rious | | | |
| WM4008MP2Mouse | TERT Chr5: 1295250 G > A | | Delete- | TERT | Promoter | het |
| Num4345 | | | rious | | SNV | |
| WM4008MP47433 | ATM:NM_000051:exon47:c.G6912T:p.E2304D | | Likely | ATM | nonsynony- | het |
| | | | Delete- | | mous SNV | |
| | | | rious | | | |
| WM4008MP47433 | BRAF:NM_004333:exon15:c.T1799A:p.V600K | | Delete- | BRAF | nonsynony- | het |
| | | | rious | | mous SNV | |
| WM4008MP47433 | PREX2:NM_024870:exon6:c.G637A:p.E213K | | Likely | PREX2 | nonsynony- | het |
| | | | Delete- | | mous SNV | |
| | | | rious | | | |
| WM4008MP47433 | RB1:NM_000321:exon25:c.A2635T:p.I879F | | Likely | RB1 | nonsynony- | het |
| | | | Delete- | | mous SNV | |
| | | | rious | | | |
| WM4008MP47433 | TERT Chr5: 1295250 G > A | | Delete- | TERT | Promoter | het |
| | | | rious | | SNV | |
| WM4018- | APC:NM_001127511:exon14:c.C8362G:p.P2788A | | Likely | APC | nonsynony- | het |
| 7MP1MouseNum0332 | | | Delete- | | mous SNV | |
| | | | rious | | | |
| WM4018- | BRAF:NM_004333:exon15:c.GT1798AA:p.V600K | | Delete- | BRAF | nonsynony- | het |
| 7MP1MouseNum0332 | | | rious | | mous SNV | |
| WM4018- | BRCA1:NM_007294:exon10:c.3891_3893del: | | Likely | BRCA1 | nonsynony- | het |
| 7MP1MouseNum0332 | p.1297_1298del | | Delete- | | mous SNV | |
| | | | rious | | | |
| WM4018- | GRM3:NM_000840:exon3:c.G835A:p.D279N | | Likely | GRM3 | nonsynony- | hom_alt |
| 7MP1MouseNum0332 | | | Delete- | | mous SNV | |
| | | | rious | | | |
| WM4018- | RAC1:NM_006908:exon2:c.C85T:p.P29S | | Delete- | RAC1 | nonsynony- | het |
| 7MP1MouseNum0332 | | | rious | | mous SNV | |
| WM4018- | TERT Chr5: 1295242 G > A | | VUS | TERT | Promoter | het |
| 7MP1MouseNum0332 | | | | | SNV | |
| WM4018- | TP53:NM_000546:exon5:c.T518G:p.V173G | | VUS | TP53 | nonsynony- | het |
| 7MP1MouseNum0332 | | | | | mous SNV | |
| WM4070- | BRAF:NM_004333:exon15:c.GT1798AA:p.V600K | | Delete- | BRAF | nonsynony- | het |
| 2MP4MouseNum6758 | | | rious | | mous SNV | |
| WM4070- | DCC:NM_005215:exon10:c.G1685A:p.R562K | | VUS | DCC | nonsynony- | het |
| 2MP4MouseNum6758 | | | | | mous SNV | |
| WM4070- | DCC:NM_005215:exon19:c.C2837T:p.S946F | | Likely | DCC | nonsynony- | het |
| 2MP4MouseNum6758 | | | Delete- | | mous SNV | |
| | | | rious | | | |
| WM4070- | DCC:NM_005215:exon2:c.G164A:p.G55E | | Likely | DCC | nonsynony- | het |
| 2MP4MouseNum6758 | | | Delete- | | mous SNV | |
| | | | rious | | | |
| WM4070- | ERBB4:NM_005235:exon2:c.A94T:p.T32S | | VUS | ERBB4 | nonsynony- | het |
| 2MP4MouseNum6758 | | | | | mous SNV | |
| WM4070- | MAP2K1:NM_002755:exon3:c.C370T:p.P124S | | Likely | MAP2K1 | nonsynony- | het |
| 2MP4MouseNum6758 | | | Delete- | | mous SNV | |
| | | | rious | | | |
| WM4070- | MAP3K9:NM_033141:exon5:c.G1294A:p.E432K | | VUS | MAP3K9 | nonsynony- | het |
| 2MP4MouseNum6758 | | | | | mous SNV | |
| WM4070- | PTPRD:NM_002839:exon20:c.G854A:p.G285E | | Likely | PTPRD | nonsynony- | het |
| 2MP4MouseNum6758 | | | Delete- | | mous SNV | |
| | | | rious | | | |
| WM4070- | TERT Chr5: 1295228 G > A | | Delete- | TERT | Promoter | het |
| 2MP4MouseNum6758 | | | rious | | SNV | |
| WM4071- | BRAF:NM_004333:exon15:c.T1799A:p.V600E | | Delete- | BRAF | nonsynony- | het |
| 1MP2MouseNum6673 | | | rious | | mous SNV | |
| WM4071- | PTEN:NM_000314:exon6:c.546dupA:p.L182fs | | Delete- | PTEN | frameshift | hom_alt |
| 1MP2MouseNum6673 | | | rious | | insertion | |
| WM4071- | TERT Chr5: 1295250 G > A | | Delete- | TERT | Promoter | het |
| 1MP2MouseNum6673 | | | rious | | SNV | |
| WM4071- | BRAF:NM_004333:exon15:c.T1799A:p.V600E | | Delete- | BRAF | nonsynony- | het |
| 1PLXMP3p5Mouse | | | rious | | mous SNV | |
| Num7328 | | | | | | |
| WM4071- | PTEN:NM_000314:exon6:c.546dupA:p.L182fs | | Delete- | PTEN | frameshift | hom_alt |
| 1PLXMP3p5Mouse | | | rious | | insertion | |
| Num7328 | | | | | | |
| WM4071- | TERT Chr5: 1295250 G > A | | Delete- | TERT | Promoter | het |
| 1PLXMP3p5Mouse | | | rious | | SNV | |
| Num7328 | | | | | | |
| WM4071-2MP2717 | BRAF:NM_004333:exon15:c.T1799A:p.V600E | | Delete- | BRAF | nonsynony- | het |
| | | | rious | | mous SNV | |
| WM4071-2MP2717 | PTEN:NM_000314:exon6:c.546dupA:p.L182fs | | Delete- | PTEN | frameshift | hom |
| | | | rious | | insertion | |
| WM4071-2MP2717 | TERT Chr5: 1295250 G > A | | Delete- | TERT | Promoter | het |
| | | | rious | | SNV | |
| WM4071- | BRAF:NM_004333:exon15:c.T1799A:p.V600E | | Delete- | BRAF | nonsynony- | het |
| 2MP2p5MouseNum7116 | | | rious | | mous SNV | |
| WM4071- | PTEN:NM_000314:exon6:c.546dupA:p.L182fs | | Delete- | PTEN | frameshift | hom |
| 2MP2p5MouseNum7116 | | | rious | | insertion | |
| WM4071- | TERT Chr5: 1295250 G > A | | Delete- | TERT | Promoter | het |
| 2MP2p5MouseNum7116 | | | rious | | SNV | |
| WM4071-2PLXMP2p6 | BRAF:NM_004333:exon15:c.T1799A:p.V600E | | Delete- | BRAF | nonsynony- | het |
| | | | rious | | mous SNV | |
| WM4071-2PLXMP2p6 | PTEN:NM_001304717:exon7:c.1065dupA:p.L355fs | | Delete- | PTEN | frameshift | hom_alt |
| | | | rious | | insertion | |
| WM4071-2PLXMP2p6 | TERT Chr5: 1295250 G > A | | Delete- | TERT | Promoter | het |
| | | | rious | | SNV | |
| WM4205-1MP2PLXp7 | ARID2:NM_152641:exon15:c.T4196A:p.L1399X | | Delete- | ARID2 | stopgain | het |
| | | | rious | | | |
| WM4205-1MP2PLXp7 | BRAF:NM_004333:exon15:c.1799_1801del: | | Likely | BRAF | nonframe- | het |
| | p.600_601del | | Delete- | | shift | |
| | | | rious | | deletion | |
| WM4205-1MP2PLXp7 | PREX2:NM_024870:exon8:c.C889T:p.R297C | | VUS | PREX2 | nonsynony- | het |
| | | | | | mous SNV | |
| WM4205-1MP2PLXp7 | TERT Chr5: 1295250 G > A | | Delete- | TERT | Promoter | het |
| | | | rious | | SNV | |
| WM4205- | ARID2:NM_152641:exon15:c.T4196A:p.L1399X | | Delete- | ARID2 | stopgain | het |
| 2PLXMP3MouseNum8835 | | | rious | | | |
| WM4205- | BRAF:NM_004333:exon15:c.1799_1801del: | | Delete- | BRAF | nonframe- | het |
| 2PLXMP3MouseNum8835 | p.600_601del (p.V600_K601delinsE) | | rious | | shift | |
| | | | | | deletion | |
| WM4205- | PREX2:NM_024870:exon8:c.C889T:p.R297C | | VUS | PREX2 | nonsynony- | het |
| 2PLXMP3MouseNum8835 | | | | | mous SNV | |
| WM4205- | TERT Chr5: 1295250 G > A | | Delete- | TERT | Promoter | het |
| 2PLXMP3MouseNum8835 | | | rious | | SNV | |
| WM4205- | ARID2:NM_152641:exon15:c.T4196A:p.L1399X | | Delete- | ARID2 | stopgain | het |
| 3MP1p4MouseNum6770 | | | rious | | | |
| WM4205- | BRAF:NM_004333:exon15:c.1799_1801del: | | Delete- | BRAF | nonframe- | hom |
| 3MP1p4MouseNum6770 | p.600_601del (p.V600_K601delinsE) | | rious | | shift | |
| | | | | | deletion | |
| WM4205- | PREX2:NM_024870:exon8:c.C889T:p.R297C | | VUS | PREX2 | nonsynony- | het |
| 3MP1p4MouseNum6770 | | | | | mous SNV | |
| WM4205- | TERT Chr5: 1295250 G > A | | Delete- | TERT | Promoter | het |
| 3MP1p4MouseNum6770 | | | rious | | SNV | |
| WM4205- | ARID2:NM_152641:exon15:c.T4196A:p.L1399X | | Delete- | ARID2 | stopgain | het |
| 3MP2MouseNum7681 | | | rious | | | |
| WM4205- | BRAF:NM_004333:exon15:c.1799_1801del: | | Delete- | BRAF | nonframe- | het |
| 3MP2MouseNum7681 | p.600_601del (p.V600_K601delinsE) | | rious | | shift | |
| | | | | | deletion | |
| WM4205- | TERT Chr5: 1295250 G > A | | Delete- | TERT | Promoter | het |
| 3MP2MouseNum7681 | | | rious | | SNV | |
| WM4205-3PLXMP2p36 | ARID2:NM_152641:exon15:c.T4196A:p.L1399X | | Delete- | ARID2 | stopgain | het |
| | | | rious | | | |
| WM4205-3PLXMP2p36 | BRAF:NM_004333:exon15:c.1799_1801del: | | Delete- | BRAF | nonframe- | hom_alt |
| | p.600_601del (p.V600_K601delinsE) | | rious | | shift | |
| | | | | | deletion | |
| WM4205-3PLXMP2p36 | TERT Chr5: 1295250 G > A | | Delete- | TERT | Promoter | het |
| | | | rious | | SNV | |
| WM4237- | ATM:NM_000051:exon55:c.C8071T:p.R2691C | | Likely | ATM | nonsynony- | hom_alt |
| 3MP1MouseNum1093 | | | Delete- | | mous SNV | |
| | | | rious | | | |
| WM4237- | BRAF:NM_004333:exon15:c.T1799A:p.V600E | | Delete- | BRAF | nonsynony- | het |
| 3MP1MouseNum1 093 | | | rious | | mous SNV | |
| WM4237- | MET:NM_001127500:exon3:c.T1286C:p.L429S | | Likely | MET | nonsynony- | het |
| 3MP1MouseNum1093 | | | Delete- | | mous SNV | |
| | | | rious | | | |
| WM4237- | PDGFRA:NM_006206:exon5:c.G723T:p.E241D | | Likely | PDGFRA | nonsynony- | hom_alt |
| 3MP1MouseNum1093 | | | Delete- | | mous SNV | |
| | | | rious | | | |
| WM4237- | RB1:NM_000321:exon20:c.2069_2082del:p.N690fs | | Delete- | RB1 | frameshift | het |
| 3MP1MouseNum1093 | | | rious | | deletion | |
| WM4237- | TERT Chr5: 1295250 G > A | | Delete- | TERT | Promoter | het |
| 3MP1MouseNum1093 | | | rious | | SNV | |
| WM4237- | TP53:NM_000546:exon7:c.C722T:p.S241F | | Delete- | TP53 | nonsynony- | hom_alt |
| 3MP1MouseNum1093 | | | rious | | mous SNV | |
| WM4237- | ATM:NM_000051:exon55:c.C8071T:p.R2691C | | Likely | ATM | nonsynony- | hom_alt |
| 4MP1MouseNum1730 | | | Delete- | | mous SNV | |
| | | | rious | | | |
| WM4237- | BRAF:NM_004333:exon15:c.T1799A:p.V600E | | Delete- | BRAF | nonsynony- | het |
| 4MP1MouseNum1730 | | | rious | | mous SNV | |
| WM4237- | MET:NM_001127500:exon3:c.T1286C:p.L429S | | Likely | MET | nonsynony- | het |
| 4MP1MouseNum1730 | | | Delete- | | mous SNV | |
| | | | rious | | | |
| WM4237- | PDGFRA:NM_006206:exon5:c.G723T:p.E241D | | Likely | PDGFRA | nonsynony- | hom_alt |
| 4MP1MouseNum1730 | | | Delete- | | mous SNV | |
| | | | rious | | | |
| WM4237- | PTPRD:NM_002839:exon43:c.C5266T:p.Q1756X | | Delete- | PTPRD | stopgain | het |
| 4MP1MouseNum1730 | | | rious | | | |
| WM4237- | RB1:NM_000321:exon20:c.2069_2082del:p.N690fs | | Delete- | RB1 | frameshift | hom_alt |
| 4MP1MouseNum1730 | | | rious | | deletion | |
| WM4237- | TERT Chr5: 1295250 G > A | | Delete- | TERT | Promoter | het |
| 4MP1MouseNum1730 | | | rious | | SNV | |
| WM4237- | TP53:NM_000546:exon7:c.C722T:p.S241F | | Delete- | TP53 | nonsynony- | hom_alt |
| 4MP1MouseNum1730 | | | rious | | mous SNV | |
| WM4237MP2p5 | ATM:NM_000051:exon55:c.C8071T:p.R2691C | | Likely | ATM | nonsynony- | hom_alt |
| | | | Delete- | | mous SNV | |
| | | | rious | | | |
| WM4237MP2p5 | BRAF:NM_004333:exon15:c.T1799A:p.V600E | | Delete- | BRAF | nonsynony- | het |
| | | | rious | | mous SNV | |
| WM4237MP2p5 | MET:NM_001127500:exon3:c.T1286C:p.L429S | | Likely | MET | nonsynony- | het |
| | | | Delete- | | mous SNV | |
| | | | rious | | | |
| WM4237MP2p5 | PDGFRA:NM_006206:exon5:c.G723T:p.E241D | | Likely | PDGFRA | nonsynony- | hom_alt |
| | | | Delete- | | mous SNV | |
| | | | rious | | | |
| WM4237MP2p5 | PTPRD:NM_002839:exon43:c.C5266T:p.Q1756X | | Delete- | PTPRD | stopgain | het |
| | | | rious | | | |
| WM4237MP2p5 | RB1:NM_000321:exon20:c.2069_2082del:p.N690fs | | Delete- | RB1 | frameshift | hom_alt |
| | | | rious | | deletion | |
| WM4237MP2p5 | TERT Chr5: 1295250 G > A | | Delete- | TERT | Promoter | het |
| | | | rious | | SNV | |
| WM4237MP2p5 | TP53:NM_000546:exon7:c.C722T:p.S241F | | Delete- | TP53 | nonsynony- | hom_alt |
| | | | rious | | mous SNV | |
| WM4239MP1Mouse | ARID1A:NM_006015:exon5:c.T1999C:p.S667P | | Likely | ARID1A | nonsynony- | hom_alt |
| Num8082 | | | Delete- | | mous SNV | |
| | | | rious | | | |
| WM4239MP1Mouse | BAP1:NM_004656:exon7:c.C505T:p.H169Y | | Likely | BAP1 | nonsynony- | het |
| Num8082 | | | Delete- | | mous SNV | |
| | | | rious | | | |
| WM4239MP1Mouse | BRAF:NM_004333:exon15:c.GT1798- | | Delete- | BRAF | nonsynony- | het |
| Num8082 | 1799AA:p.V600K | | rious | | mous SNV | |
| WM4239MP1Mouse | CDKN2A:NM_000077:exon2:c.C341T:p.P114L | | Delete- | CDKN2A | nonsynony- | hom_alt |
| Num8082 | | | rious | | mous SNV | |
| WM4239MP1Mouse | DCC:NM_005215:exon13:c.C2027A:p.P676Q | | Likely | DCC | nonsynony- | het |
| Num8082 | | | Delete- | | mous SNV | |
| | | | rious | | | |
| WM4239MP1Mouse | MET:NM_001127500:exon2:c.G1132A:p.V378I | | Likely | MET | nonsynony- | het |
| Num8082 | | | Delete- | | mous SNV | |
| | | | rious | | | |
| WM4239MP1Mouse | TERT Chr5: 1295250 G > A | | Delete- | TERT | Promoter | het |
| Num8082 | | | rious | | SNV | |
| WM4249MP1Mouse | APC:NM_001127511:exon14:c.C3329T:p.S1110F | | VUS | APC | nonsynony- | het |
| Num8724 | | | | | mous SNV | |
| WM4249MP1Mouse | BRAF:NM_004333:exon15:c.T1799A:p.V600E | | Delete- | BRAF | nonsynony- | het |
| Num8724 | | | rious | | mous SNV | |
| WM4249MP1Mouse | DCC:NM_005215:exon2:c.G164A:p.G55E | | VUS | DCC | nonsynony- | het |
| Num8724 | | | | | mous SNV | |
| WM4249MP1Mouse | DDX3X:NM_001356:exon8:c.T730G:p.S244A | | VUS | DDX3X | nonsynony- | het |
| Num8724 | | | | | mous SNV | |
| WM4249MP1Mouse | GRIN2A:NM_001134407:exon13:c.C4238T:p.S1413F | | Likely | GRIN2A | nonsynony- | het |
| Num8724 | | | Delete- | | mous SNV | |
| | | | rious | | | |
| WM4249MP1Mouse | JAK3:NM_000215:exon24:c.G3233A:p.W1078X | | VUS | JAK3 | stopgain | het |
| Num8724 | | | | | | |
| WM4249MP1Mouse | MAP2K2:NM_030662:exon7:c.C889T:p.R297W | | Likely | MAP2K2 | nonsynony- | het |
| Num8724 | | | Delete- | | mous SNV | |
| | | | rious | | | |
| WM4249MP1Mouse | NRAS:NM_002524:exon2:c.C67A:p.L23I | | Likely | NRAS | nonsynony- | het |
| Num8724 | | | Delete- | | mous SNV | |
| | | | rious | | | |
| WM4249MP1Mouse | PDGFRA:NM_006206:exon8:c.G1156A:p.E386K | | VUS | PDGFRA | nonsynony- | het |
| Num8724 | | | | | mous SNV | |
| WM4249MP1Mouse | PREX2:NM_024870:exon19:c.G2107A:p.G703R | | VUS | PREX2 | nonsynony- | het |
| Num8724 | | | | | mous SNV | |
| WM4249MP1Mouse | PTPRD:NM_002839:exon24:c.C1825T:p.P609S | | Likely | PTPRD | nonsynony- | het |
| Num8724 | | | Delete- | | mous SNV | |
| | | | rious | | | |
| WM4249MP1Mouse | PTPRD:NM_002839:exon28:c.C2735T:p.S912F | | Likely | PTPRD | nonsynony- | het |
| Num8724 | | | Delete- | | mous SNV | |
| | | | rious | | | |
| WM4249MP1Mouse | RB1:NM_000321:exon26:c.G2678C:p.G893A | | VUS | RB1 | nonsynony- | het |
| Num8724 | | | | | mous SNV | |
| WM4249MP1Mouse | SETD2:NM_014159:exon8:c.C4970T:p.P1657L | | Likely | SETD2 | nonsynony- | het |
| Num8724 | | | Delete- | | mous SNV | |
| | | | rious | | | |
| WM4249MP1Mouse | SMARCA4:NM_001128849:exon19:c.T2827C:p.F943L | | Likely | SMARCA4 | nonsynony- | het |
| Num8724 | | | Delete- | | mous SNV | |
| | | | rious | | | |
| WM4249MP1Mouse | SOX10:NM_006941:exon3:c.G472A:p.E158K | | Likely | SOX10 | nonsynony- | het |
| Num8724 | | | Delete- | | mous SNV | |
| | | | rious | | | |
| WM4249PLXMP2PLXp7 | APC:NM_001127511:exon14:c.C3329T:p.S1110F | | VUS | APC | nonsynony- | het |
| MouseNum8638 | | | | | mous SNV | |
| WM4249PLXMP2PLXp7 | BRAF:NM_004333:exon15:c.T1799A:p.V600E | | Delete- | BRAF | nonsynony- | het |
| MouseNum8638 | | | rious | | mous SNV | |
| WM4249PLXMP2PLXp7 | DCC:NM_005215:exon2:c.G164A:p.G55E | | Likely | DCC | nonsynony- | het |
| MouseNum8638 | | | Delete- | | mous SNV | |
| | | | rious | | | |
| WM4249PLXMP2PLXp7 | GRIN2A:NM_001134407:exon13:c.C4238T:p.S1413F | | Likely | GRIN2A | nonsynony- | het |
| MouseNum8638 | | | Delete- | | mous SNV | |
| | | | rious | | | |
| WM4249PLXMP2PLXp7 | JAK3:NM_000215:exon24:c.G3233A:p.W1078X | | VUS | JAK3 | stopgain | het |
| MouseNum8638 | | | | | | |
| WM4249PLXMP2PLXp7 | MAP2K2:NM_030662:exon7:c.C889T:p.R297W | | Likely | MAP2K2 | nonsynony- | het |
| MouseNum8638 | | | Delete- | | mous SNV | |
| | | | rious | | | |
| WM4249PLXMP2PLXp7 | PDGFRA:NM_006206:exon8:c.G1156A:p.E386K | | VUS | PDGFRA | nonsynony- | het |
| MouseNum8638 | | | | | mous SNV | |
| WM4249PLXMP2PLXp7 | PREX2:NM_024870:exon19:c.G2107A:p.G703R | | Likely | PREX2 | nonsynony- | het |
| MouseNum8638 | | | Delete- | | mous SNV | |
| | | | rious | | | |
| WM4249PLXMP2PLXp7 | PTPRD:NM_002839:exon24:c.C1825T:p.P609S | | Likely | PTPRD | nonsynony- | het |
| MouseNum8638 | | | Delete- | | mous SNV | |
| | | | rious | | | |
| WM4249PLXMP2PLXp7 | PTPRD:NM_002839:exon28:c.C2735T:p.S912F | | Likely | PTPRD | nonsynony- | het |
| MouseNum8638 | | | Delete- | | mous SNV | |
| | | | rious | | | |
| WM4249PLXMP2PLXp7 | SETD2:NM_014159:exon8:c.C4970T:p.P1657L | | Likely | SETD2 | nonsynony- | het |
| MouseNum8638 | | | Delete- | | mous SNV | |
| | | | rious | | | |
| WM4249PLXMP2PLXp7 | SMARCA4:NM_001128849:exon19:c.T2827C:p.F943L | | Likely | SMARCA4 | nonsynony- | het |
| MouseNum8638 | | | Delete- | | mous SNV | |
| | | | rious | | | |
| WM4249PLXMP2PLXp7 | SOX10:NM_006941:exon3:c.G472A:p.E158K | | Likely | SOX10 | nonsynony- | het |
| MouseNum8638 | | | Delete- | | mous SNV | |
| | | | rious | | | |
| WM4258- | BRAF:NM_004333:exon15:c.T1799A:p.V600E | | Delete- | BRAF | nonsynony- | het |
| 2MP1MouseNum1596 | | | rious | | mous SNV | |
| WM4258- | FGFR3:NM_000142:exon18:c.C2291T:p.S764L | | Likely | FGFR3 | nonsynony- | hom_alt |
| 2MP1MouseNum1596 | | | Delete- | | mous SNV | |
| | | | rious | | | |
| WM4258- | PRC1:NM_003981:exon3:c.T206C:p.I69T | | Likely | PRC1 | nonsynony- | het |
| 2MP1MouseNum1596 | | | Delete- | | mous SNV | |
| | | | rious | | | |
| WM4258- | RB1:NM_000321:exon14:c.C1363T:p.R455X | | Delete- | RB1 | stopgain | hom_alt |
| 2MP1MouseNum1596 | | | rious | | | |
| WM4258- | TERT Chr5: 1295228 G > A | | Delete- | TERT | Promoter | hom_alt |
| 2MP1MouseNum1596 | | | rious | | SNV | |
| WM4258- | TP53:NM_000546:exon7:c.673 − 1G > A | | Likely | TP53 | splicing | hom_alt |
| 2MP1MouseNum1596 | | | Delete- | | | |
| | | | rious | | | |
| WM4262CPLXMP2Mouse | ATM:NM_000051:exon33:c.C4964T:p.S1655F | | VUS | ATM | nonsynony- | hom_alt |
| Num8592 | | | | | mous SNV | |
| WM4262CPLXMP2Mouse | BRAF:NM_004333:exon15:c.T1799A:p.V600E | | Delete- | BRAF | nonsynony- | hom_alt |
| Num8592 | | | rious | | mous SNV | |
| WM4262CPLXMP2Mouse | MYC:NM_002467:exon2:c.C85T:p.L29F | | Likely | MYC | nonsynony- | het |
| Num8592 | | | Delete- | | mous SNV | |
| | | | rious | | | |
| WM4262CPLXMP2Mouse | NRAS:NM_002524:exon2:c.G37C:p.G13R | | Delete- | NRAS | nonsynony- | het |
| Num8592 | | | rious | | mous SNV | |
| WM4262CPLXMP2Mouse | PPP6C:NM_001123355:exon5:c.C451T:p.H151Y | | Likely | PPP6C | nonsynony- | het |
| Num8592 | | | Delete- | | mous SNV | |
| | | | rious | | | |
| WM4262CPLXMP2Mouse | PREX2:NM_024870:exon34:c.C4210T:p.H1404Y | | VUS | PREX2 | nonsynony- | het |
| Num8592 | | | | | mous SNV | |
| WM4262CPLXMP2Mouse | PTPRD:NM_002839:exon32:c.C3599T:p.P1200L | | Likely | PTPRD | nonsynony- | het |
| Num8592 | | | Delete- | | mous SNV | |
| | | | rious | | | |
| WM4262CPLXMP2Mouse | TERT Chr5: 1295228 G > A | | Delete- | TERT | Promoter | het |
| Num8592 | | | rious | | SNV | |
| WM4262CPLXMP2Mouse | WT1:NM_000378:exon1:c.C494T:p.S165F | | Likely | WT1 | nonsynony- | het |
| Num8592 | | | Delete- | | mous SNV | |
| | | | rious | | | |
| WM4262MP1p5Mouse | ATM:NM_000051:exon33:c.C4964T:p.S1655F | | VUS | ATM | nonsynony- | hom_alt |
| Num8539 | | | | | mous SNV | |
| WM4262MP1p5Mouse | BRAF:NM_004333:exon15:c.T1799A:p.V600E | | Delete- | BRAF | nonsynony- | hom_alt |
| Num8539 | | | rious | | mous SNV | |
| WM4262MP1p5Mouse | MYC:NM_002467:exon2:c.C85T:p.L29F | | Likely | MYC | nonsynony- | het |
| Num8539 | | | Delete- | | mous SNV | |
| | | | rious | | | |
| WM4262MP1p5Mouse | NRAS:NM_002524:exon2:c.G37C:p.G13R | | Delete- | NRAS | nonsynony- | het |
| Num8539 | | | rious | | mous SNV | |
| WM4262MP1p5Mouse | PPP6C:NM_001123355:exon5:c.C451T:p.H151Y | | Likely | PPP6C | nonsynony- | het |
| Num8539 | | | Delete- | | mous SNV | |
| | | | rious | | | |
| WM4262MP1p5Mouse | PREX2:NM_024870:exon34:c.C4210T:p.H1404Y | | VUS | PREX2 | nonsynony- | het |
| Num8539 | | | | | mous SNV | |
| WM4262MP1p5Mouse | PTPRD:NM_002839:exon32:c.C3599T:p.P1200L | | Likely | PTPRD | nonsynony- | het |
| Num8539 | | | Delete- | | mous SNV | |
| | | | rious | | | |
| WM4262MP1p5Mouse | TERT Chr5: 1295228 G > A | | Delete- | TERT | Promoter | het |
| Num8539 | | | rious | | SNV | |
| WM4262MP1p5Mouse | WT1:NM_000378:exon1:c.C494T:p.S165F | | Likely | WT1 | nonsynony- | het |
| Num8539 | | | Delete- | | mous SNV | |
| | | | rious | | | |
| WM4264- | ABL1:NM_005157:exon11:c.C2329T:p.R777X | | VUS | ABL1 | stopgain | het |
| 1MP1MouseNum8243 | | | | | | |
| WM4264- | APC:NM_001127511:exon14:c.G4672T:p.E1558X | | VUS | APC | stopgain | hom_alt |
| 1MP1MouseNum8243 | | | | | | |
| WM4264- | ATM:NM_000051:exon30:c.C4465T:p.R1489C | | Likely | ATM | nonsynony- | het |
| 1MP1MouseNum8243 | | | Delete- | | mous SNV | |
| | | | rious | | | |
| WM4264- | BRAF:NM_004333:exon15:c.T1799A:p.V600E | | Delete- | BRAF | nonsynony- | het |
| 1MP1MouseNum8243 | | | rious | | mous SNV | |
| WM4264- | TERT Chr5: 1295228 G > A | | Delete- | TERT | Promoter | het |
| 1MP1MouseNum8243 | | | rious | | SNV | |
| WM4264- | TRRAP:NM_001244580:exon45:c.G6622T:p.V2208L | | VUS | TRRAP | nonsynony- | het |
| 1MP1MouseNum8243 | | | | | mous SNV | |
| WM4264- | ABL1:NM_005157:exon11:c.C2329T:p.R777X | | VUS | ABL1 | stopgain | het |
| 2MP1MouseNum9997 | | | | | | |
| WM4264- | ATM:NM_000051:exon30:c.C4465T:p.R1489C | | Likely | ATM | nonsynony- | het |
| 2MP1MouseNum9997 | | | Delete- | | mous SNV | |
| | | | rious | | | |
| WM4264- | BRAF:NM_004333:exon15:c.T1799A:p.V600E | | Delete- | BRAF | nonsynony- | het |
| 2MP1MouseNum9997 | | | rious | | mous SNV | |
| WM4264- | MAP2K2:NM_030662:exon2:c.A181G:p.K61E | | VUS | MAP2K2 | nonsynony- | het |
| 2MP1MouseNum9997 | | | | | mous SNV | |
| WM4264- | MET:NM_001127500:exon2:c.T721C:p.F241L | | VUS | MET | nonsynony- | het |
| 2MP1MouseNum9997 | | | | | mous SNV | |
| WM4264- | TERT Chr5: 1295228 G > A | | Delete- | TERT | Promoter | het |
| 2MP1MouseNum9997 | | | rious | | SNV | |
| WM4264- | TRRAP:NM_001244580:exon45:c.G6622T:p.V2208L | | VUS | TRRAP | nonsynony- | het |
| 2MP1MouseNum9997 | | | | | mous SNV | |
| WM4280PLXMP1Mouse | BRAF:NM_004333:exon15:c.GT1798AA:p.V600K | | Delete- | BRAF | nonsynony- | het |
| Num9005 | | | rious | | mous SNV | |
| WM4280PLXMP1Mouse | GRIN2A:NM_001134407:exon12:c.G2364T:p.M7881 | | Likely | GRIN2A | nonsynony- | het |
| Num9005 | | | Delete- | | mous SNV | |
| | | | rious | | | |
| WM4280PLXMP1Mouse | IDH1:NM_005896:exon4:c.C394T:p.R132C | | Delete- | IDH1 | nonsynony- | het |
| Num9005 | | | rious | | mous SNV | |
| WM4280PLXMP1Mouse | NRAS:NM_002524:exon2:c.G34C:p.G12R | | Delete- | NRAS | nonsynony- | het |
| Num9005 | | | rious | | mous SNV | |
| WM4280PLXMP1Mouse | PPP6C:NM_001123355:exon5:c.C451T:p.H151Y | | Likely | PPP6C | nonsynony- | het |
| Num9005 | | | Delete- | | mous SNV | |
| | | | rious | | | |
| WM4280PLXMP1Mouse | PREX1:NM_020820:exon28:c.C3632T:p.P1211L | | Likely | PREX1 | nonsynony- | het |
| Num9005 | | | Delete- | | mous SNV | |
| | | | rious | | | |
| WM4280PLXMP1Mouse | PTCH1:NM_001083602:exon2:c.G146C:p.G49A | | Likely | PTCH1 | nonsynony- | het |
| Num9005 | | | Delete- | | mous SNV | |
| | | | rious | | | |
| WM4280PLXMP1Mouse | TERT Chr5: 1295228 G > A | | Delete- | TERT | Promoter | het |
| Num9005 | | | rious | | SNV | |
| WM4285MP1Mouse | ALK:NM_004304:exon28:c.A4115G:p.D1372G | | Likely | ALK | nonsynony- | het |
| Num9571 | | | Delete- | | mous SNV | |
| | | | rious | | | |
| WM4285MP1Mouse | APC:NM_001127511:exon14:c.C6995T:p.S2332F | | Likely | APC | nonsynony- | het |
| Num9571 | | | Delete- | | mous SNV | |
| | | | rious | | | |
| WM4285MP1Mouse | BRAF:NM_004333:exon15:c.T1799A:p.V600E | | Delete- | BRAF | nonsynony- | het |
| Num9571 | | | rious | | mous SNV | |
| WM4285MP1Mouse | ERBB4:NM_005235:exon18:c.C2131T:p.R711C | | Likely | ERBB4 | nonsynony- | het |
| Num9571 | | | Delete- | | mous SNV | |
| | | | rious | | | |
| WM4285MP1Mouse | FGFR2:NM_000141:exon3:c.A129C:p.Q43H | | Likely | FGFR2 | nonsynony- | het |
| Num9571 | | | Delete- | | mous SNV | |
| | | | rious | | | |
| WM4285MP1Mouse | PTPRD:NM_002839:exon32:c.A3574G:p.I1192V | | VUS | PTPRD | nonsynony- | het |
| Num9571 | | | | | mous SNV | |
| WM4285MP1Mouse | TERT Chr5: 1295250 G > A | | Delete- | TERT | Promoter | het |
| Num9571 | | | rious | | SNV | |
| WM4335MP1Mouse | BRAF:NM_004333:exon15:c.T1799A:p.V600E | | Delete- | BRAF | nonsynony- | het |
| Num1387 | | | rious | | mous SNV | |
| WM4335MP1Mouse | BRCA2:NM_000059:exon13:c.6942_6945del:p.T2314fs | | Delete- | BRCA2 | frameshift | het |
| Num1387 | | | rious | | deletion | |
| WM4335MP1Mouse | EGFR:NM_005228:exon18:c.G2065C:p.V689L | | Likely | EGFR | nonsynony- | het |
| Num1387 | | | Delete- | | mous SNV | |
| | | | rious | | | |
| WM4335MP1Mouse | FBXW7:NM_033632:exon6:c.861 + 1G > A | | Likely | FBXW7 | splicing | het |
| Num1387 | | | Delete- | | | |
| | | | rious | | | |
| WM4335MP1Mouse | GRM3:NM_000840:exon4:c.G2230A:p.D744N | | VUS | GRM3 | nonsynony- | het |
| Num1387 | | | | | mous SNV | |
| WM4335MP1Mouse | KDR:NM_002253:exon3:c.G289A:p.D97N | | Likely | KDR | nonsynony- | het |
| Num1387 | | | Delete- | | mous SNV | |
| | | | rious | | | |
| WM4335MP1Mouse | MAP3K9:NM_033141:exon2:c.C523T:p.H175Y | | VUS | MAP3K9 | nonsynony- | het |
| Num1387 | | | | | mous SNV | |
| WM4335MP1Mouse | PREX2:NM_024870:exon37:c.C4423T:p.P1475S | | VUS | PREX2 | nonsynony- | het |
| Num1387 | | | | | mous SNV | |
| WM4335MP1Mouse | ROS1:NM_002944:exon35:c.G5713A:p.E1905K | | VUS | ROS1 | nonsynony- | het |
| Num1387 | | | | | mous SNV | |
| WM4335MP1Mouse | TP53:NM_000546:exon5:c.G524A:p.R175H | | Delete- | TP53 | nonsynony- | hom_alt |
| Num1387 | | | rious | | mous SNV | |
| WM4336- | BRAF:NM_004333:exon15:c.T1799A:p.V600E | | Delete- | BRAF | nonsynony- | het |
| 1MP1MouseNum555 | | | rious | | mous SNV | |
| WM4336- | DCC:NM_005215:exon18:c.G2749A:p.E917K | | Likely | DCC | nonsynony- | het |
| 1MP1MouseNum555 | | | Delete- | | mous SNV | |
| | | | rious | | | |
| WM4336- | PTEN:NM_001304717:exon4:c.684 − 1G > A | | Likely | PTEN | splicing | hom_alt |
| 1MP1MouseNum555 | | | Delete- | | | |
| | | | rious | | | |
| WM4360MP1Mouse | BRAF:NM_004333:exon15:c.T1799A:p.V600E | | Delete- | BRAF | nonsynony- | het |
| Num1020 | | | rious | | mous SNV | |
| WM4360MP1Mouse | CDKN2A:NM_000077:exon2:c.151 − 1G > C | | Likely | CDKN2A | splicing | hom_alt |
| Num1020 | | | Delete- | | | |
| | | | rious | | | |
| WM4360MP1Mouse | ERBB4:NM_005235:exon13:c.C1565T:p.S522L | | VUS | ERBB4 | nonsynony- | het |
| Num1020 | | | | | mous SNV | |
| WM4360MP1Mouse | NOTCH2:NM_024408:exon6:c.G958A:p.G320R | | Likely | NOTCH2 | nonsynony- | het |
| Num1020 | | | Delete- | | mous SNV | |
| | | | rious | | | |
| WM4360MP1Mouse | PDGFRA:NM_006206:exon12:c.G1702A:p.D568N | | Likely | PDGFRA | nonsynony- | het |
| Num1020 | | | Delete- | | mous SNV | |
| | | | rious | | | |
| WM4360MP1Mouse | TERT Chr5: 1295228 G > A | | Delete- | TERT | nonsynony- | het |
| Num1020 | | | rious | | mous SNV | |
| WM4388MP1Mouse | BRAF:NM_004333:exon15:c.T1799A:p.V600E | | Delete- | BRAF | nonsynony- | het |
| Num1808 | | | rious | | mous SNV | |
| WM4388MP1Mouse | CDKN2A:NM_000077:exon1:c.28_40del:p.E10fs | | Delete- | CDKN2A | frameshift | het |
| Num1808 | | | rious | | deletion | |
| WM4388MP1Mouse | TERT Chr5: 1295228-1295229 GG > AA | | Delete- | TERT | Promoter | het |
| Num1808 | | | rious | | SNV | |
| WM4388MP1Mouse | TP53:NM_000546:exon6:c.C637T:p.R213X | | Delete- | TP53 | stopgain | hom_alt |
| Num1808 | | | rious | | | |
| WM4390MP1Mouse | BRAF:NM_004333:exon15:c.T1799A:p.V600E | | Delete- | BRAF | nonsynony- | het |
| Num1827 | | | rious | | mous SNV | |
| WM4390MP1Mouse | CCND2:NM_001759:exon2:c.C281T:p.P94L | | VUS | CCND2 | nonsynony- | het |
| Num1827 | | | | | mous SNV | |
| WM4390MP1Mouse | GNA11:NM_002067:exon4:c.C547T:p.R183C | | Likely | GNA11 | nonsynony- | het |
| Num1827 | | | Delete- | | mous SNV | |
| | | | rious | | | |
| WM4398MP1Mouse | BRAF:NM_004333:exon15:c.T1799A:p.V600E | | Delete- | BRAF | nonsynony- | het |
| Num1722 | | | rious | | mous SNV | |
| WM4398MP1Mouse | DCC:NM_005215:exon16:c.C2371T:p.H791Y | | Likely | DCC | nonsynony- | hom_alt |
| Num1722 | | | Delete- | | mous SNV | |
| | | | rious | | | |
| WM4398MP1Mouse | MET:NM_001127500:exon9:c.C2181A:p.D727E | | Likely | MET | nonsynony- | het |
| Num1722 | | | Delete- | | mous SNV | |
| | | | rious | | | |
| WM4398MP1Mouse | PRDM2:NM_012231:exon3:c.G106A:p.A36T | | Likely | PRDM2 | nonsynony- | het |
| Num1722 | | | Delete- | | mous SNV | |
| | | | rious | | | |
| WM4398MP1Mouse | TERT Chr5: 1295250 G > A | | Delete- | TERT | Promoter | het |
| Num1722 | | | rious | | SNV | |
| WM4408MP1Mouse | ALK:NM_004304:exon29:c.C4774T:p.P1592S | | Likely | ALK | nonsynony- | het |
| Num1965 | | | Delete- | | mous SNV | |
| | | | rious | | | |
| WM4408MP1Mouse | ARID1A:NM_006015:exon1:c.C581T:p.P194L | | VUS | ARID1A | nonsynony- | hom_alt |
| Num1965 | | | | | mous SNV | |
| WM4408MP1Mouse | BRAF:NM_004333:exon15:c.T1799A:p.V600E | | Delete- | BRAF | nonsynony- | het |
| Num1965 | | | rious | | mous SNV | |
| WM4408MP1Mouse | DCC:NM_005215:exon13:c.C1951G:p.Q651E | | Likely | DCC | nonsynony- | hom_alt |
| Num1965 | | | Delete- | | mous SNV | |
| | | | rious | | | |
| WM4408MP1Mouse | TERT Chr5: 1295250 G > A | | Delete- | TERT | Promoter | het |
| Num1965 | | | rious | | SNV | |

Given the prevalence of TERT promoter mutations in melanoma and the potency of telomerase-directed 6-thio-dG therapy in inhibiting cell proliferation of telomerase activity-positive lung and colon cancer cells, the efficacy of 6-thio-dG in the BRAF-mutant melanoma subtype was tested. To that end, a panel of 12 BRAF-mutant human metastatic melanoma cell lines were treated with 6-thio-dG and BIBR 1532 for 9-12 days, respectively. Similarly, the anti-proliferative effect of 6-thio-dG in BRAF-mutant melanoma cell lines was observed (FIG. 1B).

Treatment of BRAF-mutant Melanoma Cells with 6-thio-dG Impairs Cell Viability and Tumor Growth.

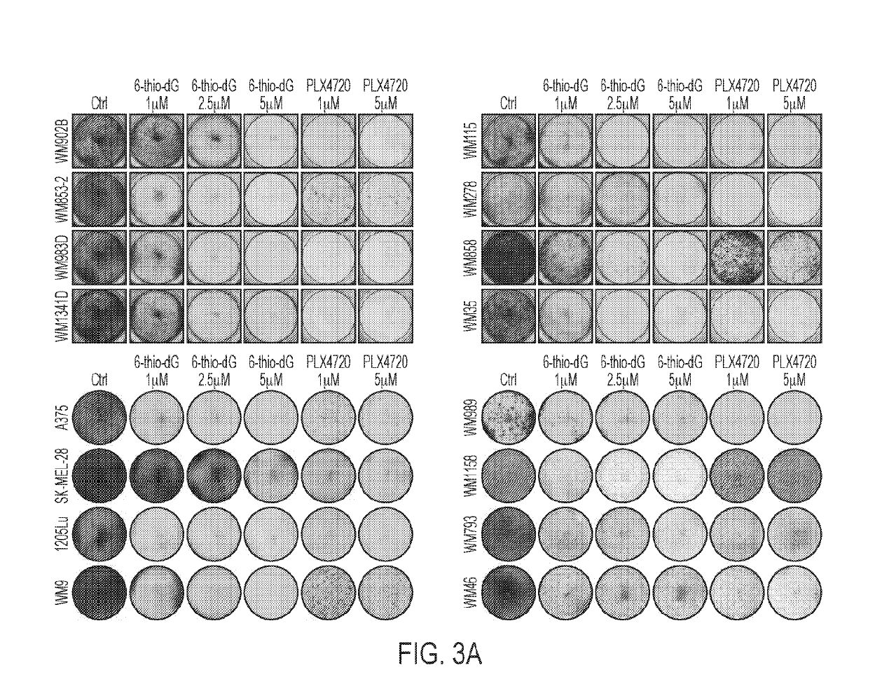

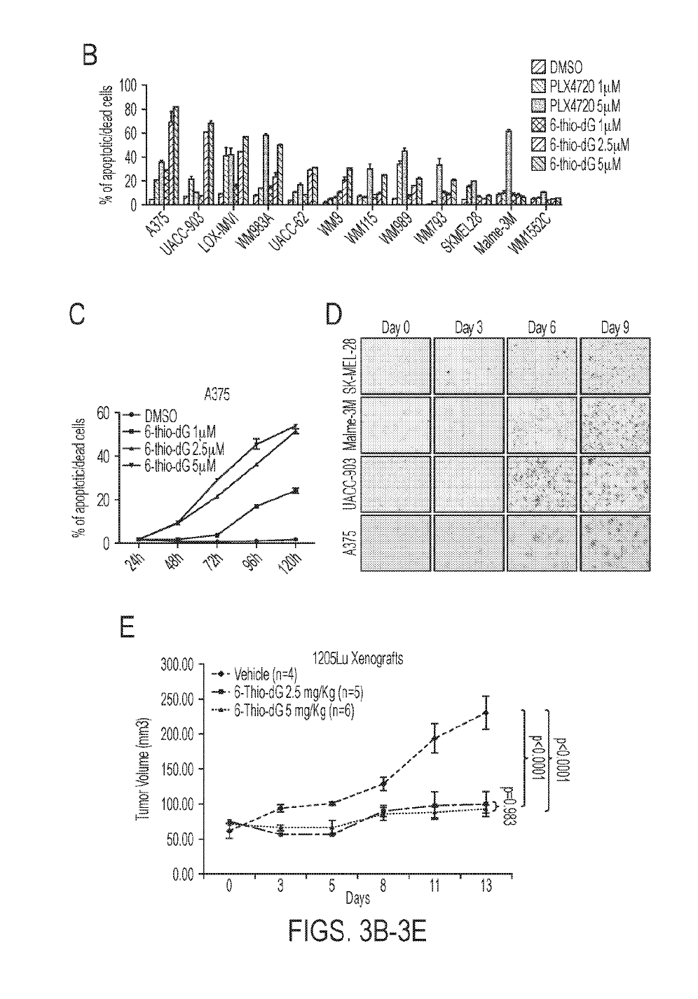

By investigating BRAF-mutant melanoma cell lines, the efficacy of 6-thio-dG was compared with that of the BRAF inhibitor, PLX4720. Another cohort of 16 BRAF-mutant melanoma cell lines was treated with 6-thio-dG and PLX4270 for 9 to 12 days, respectively. It was demonstrated that 6-thio-dG substantially inhibited cell proliferation. The efficacy of 6-thio-dG was comparable to and in some cases even superior to that of PLX4720 (FIG. 3A).

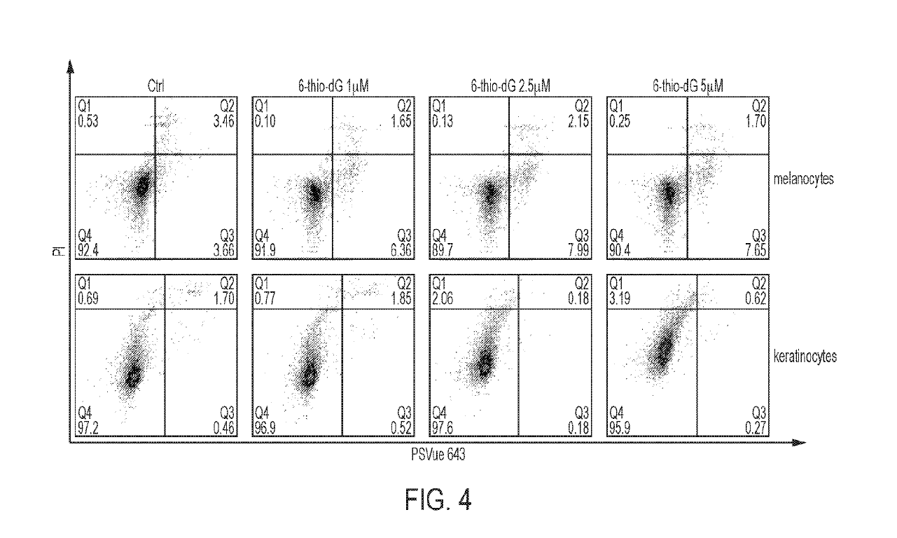

Treatment with 6-thio-dG over a shorter period of 5 days further revealed that 6-thio-dG significantly induced apoptosis and cell death in 9 of 12 BRAF-mutant melanoma cell lines (FIG. 3B). A375 melanoma cells was most sensitive to 6-thio-dG among all 12 BRAF-mutant melanoma cell lines (FIG. 3B). In a time-course experiment, it was shown that the induction of apoptosis started to occur as early as 48 hour post-treatment (FIG. 3C). A previous study showed that normal fibroblasts were not sensitive to 6-thio-dG, which is in line with the finding that 6-thio-dG did not impair the cell viability of normal epidermal melanocytes and keratinocytes (FIG. 4) (Mender et al., 2015).

The induction of apoptosis and cell death prompted investigation of cells that had survived 6-thio-dG. The senescence-associated β-gal (SA-β-gal) staining of 4 representative BRAF-mutant melanoma cell lines suggested that the long-term treatment with 6-thio-dG triggered the induction of cellular senescence in cells that survived the initial killing by 6-thio-dG (FIG. 3D). Finally, using xenografts of the human metastatic melanoma 1205Lu cell line, it was shown that 6-thio-dG was able to significantly impair tumor growth (FIG. 3E).

Gene Expression and Protein Expression Signatures of 6-Thio-dG.

To gain mechanistic insights into the anti-proliferative effect of 6-thio-dG, A375 cells were treated with the control, BIBR 1532, and 6-thio-dG for 4 days, respectively. Integrated analyses were then carried out to profile the transcriptome with RNA sequencing (RNA-seq) and the functional proteome with reverse phase protein array (RPPA).

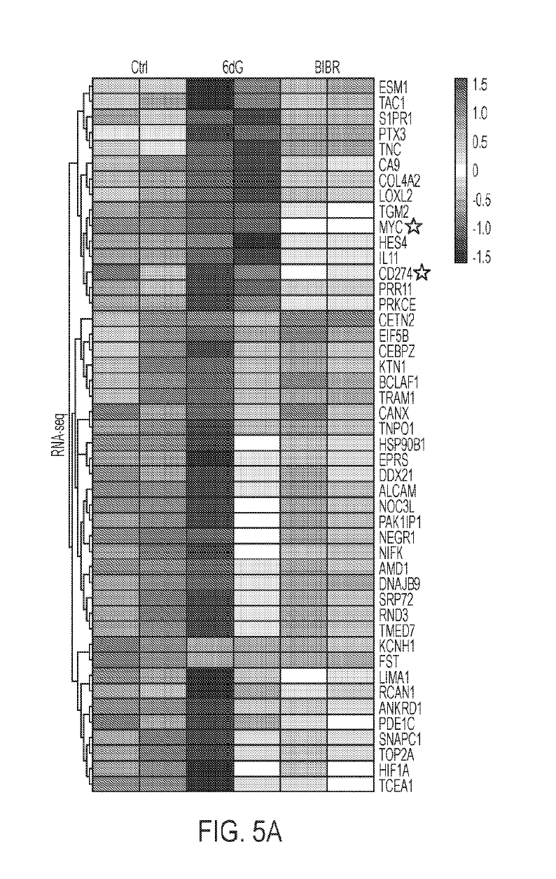

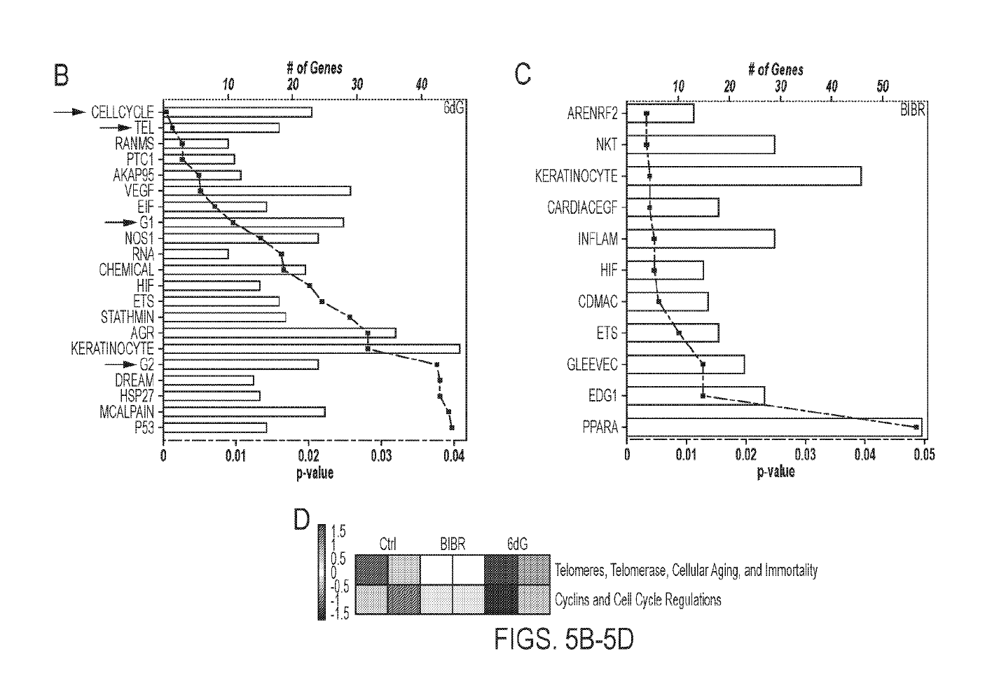

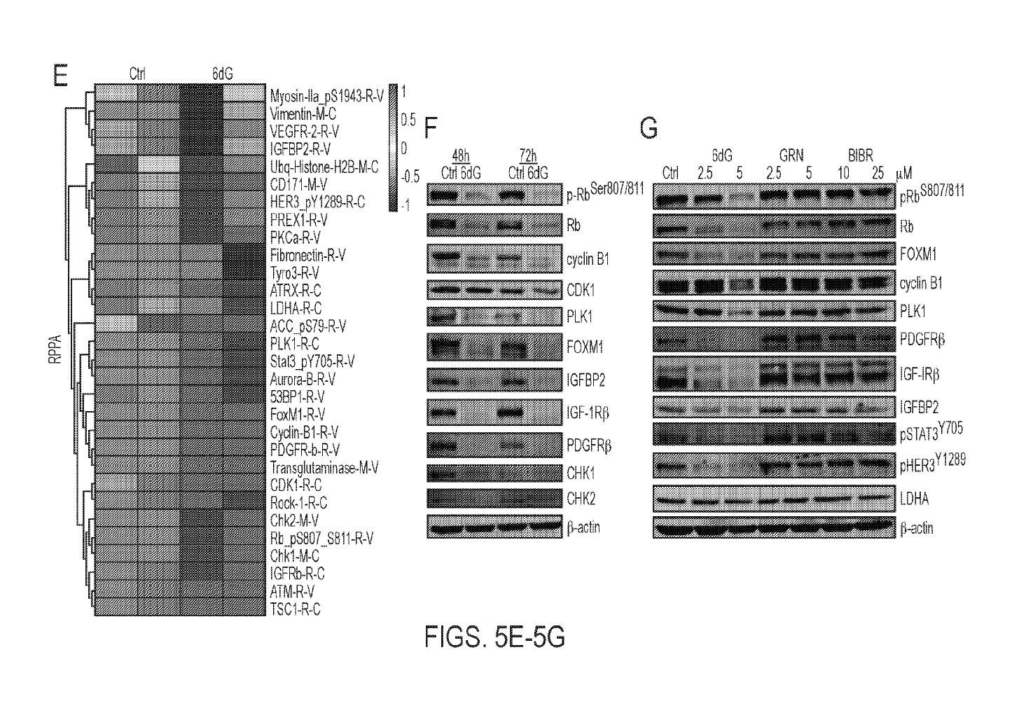

The differential expression analysis identified genes that were significantly down-regulated by 6-thio-dG, among which CD274 (PD-L1) and c-Myc showed the highest degrees of change (FIG. 5A). To further unravel which signaling pathways were altered in A375 cells treated with 6-thio-dG, the hypergeometric differential analysis was carried out to identify the pathways that were statistically enriched among the significantly down-regulated genes. This analysis demonstrated that BioCarta gene sets related to cell cycle and telomere biology were among the top 10 ranked pathways (FIGS. 5B-5C). Furthermore, a complementary approach—single-sample gene set enrichment analysis (ssGSEA)—was utilized to verify that “cell cycle” and “telomere signaling” pathways were indeed suppressed by 6-thio-dG (FIG. 5D).

The analysis of RPPA data identified the top 30 significantly down-regulated proteins in A375 cells treated with 6-thio-dG. It was concluded that three major signaling pathways were altered in A375 cells treated with 6-thio-dG. They were (1) cell cycle: phospho-RB^{Ser807/811}, cyclin B1, CDK1, FOXM1, PLK1 and AURKB; (2) DNA damage response: 53BP1, ATM, ATR, CHK1 and CHK2; and (3) receptor tyrosine kinase signaling: VEGFR, PDGFRb, IGF-IRb, IGFBP2, phospho-STAT3^{Y705} and phospho-HER3^{Y1289} (FIG. 5E). Most of the down-regulated proteins were validated by western blotting and showed that these proteins were indeed inhibited in A375 cells treated with 6-thio-dG but not BIBR 1532 or another telomerase inhibitor GRN163L (FIGS. 5F-5G).

Because the computational analysis indicated that the cell cycle pathway is altered, A375 cells that express the “fluorescent, ubiquitination-based, cell-cycle indicators Fucci, version 2” (A375^{Fucci2}) were further exploited in order to visualize cell cycle progression. The time-lapse imaging showed that A375^{Fucci2} cells treated with 6-thio-dG over 42 hours were arrested at the G₂/M checkpoint followed by cell death (data not shown).

6-Thio-dG Down-Regulates PLK1, PDGFRβ, ARIDIA and AXL at the Protein Level.

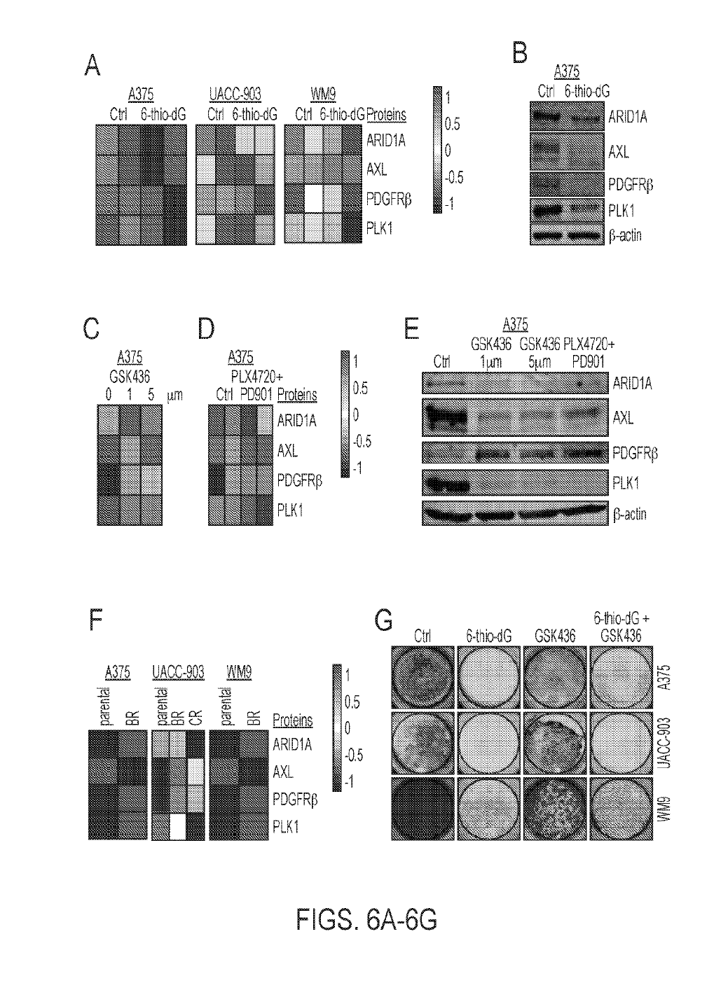

To identify signaling protein molecules that were commonly altered in melanoma cells treated with 6-thio-dG, RPPA was carried out to profile 3 BRAF-mutant melanoma cell lines, including A375, UACC-903 and WM9. The analysis of RPPA data showed that PLK1, PDGFRβ, ARIDIA and AXL were significantly down-regulated at the protein level in all three cell lines (FIG. 6A), which was further validated with A375 cells treated with 6-thio-dG by western blotting (FIG. 6B).

Notably, AXL and PDGFRβ are known to mediate intrinsic and acquired resistance of BRAF-mutant melanoma cells to MAPKi (Muller et al., 2014; Nazarian et al., 2010; Tirosh et al., 2016). Indeed, the analysis of RPPA data showed that PDGFRβ was up-regulated in A375 cells that were intrinsically resistant to the short-term treatment with MAPKi (FIG. 6C-6D), which was validated by the western blotting of A375 cells treated with MAPKi (FIG. 6E). Moreover, ARIDIA, PDGFRβ and PLK1 but not AXL were up-regulated in A375 and WM9 cells that acquired resistance to PLX4720 (FIG. 6F). Similarly, AXL and PDGFRβ were up-regulated in UACC-903 cells that acquired resistance to PLX4720 or the combination of PLX4720 and PD0325901 (FIG. 6F).

This data prompted further experimentation to test whether the combination of 6-thio-dG with the BRAF inhibitor could delay or abrogate the acquisition of therapy resistance to BRAFi. Indeed, the combination of 6-thio-dG with GSK2118436 (GSK436; dabrafenib) substantially inhibited the emergence of clones in all three BRAF-mutant melanoma cell lines that acquired resistance to GSK2118436 (FIG. 6G).

6-Thio-dG Significantly Impairs Cell Viability, Proliferation and Tumor Growth of MAPKi Resistant Melanoma Cells.

AXL and PDGFRβ are known to mediate the acquired resistance to MAPKi, therefore the efficacy of 6-thio-dG in MAPKi-resistant melanoma cell lines was tested, due to its ability to inhibit ARIDIA, AXL, PDGFRβ and PLK and to overcome the intrinsic drug resistance.

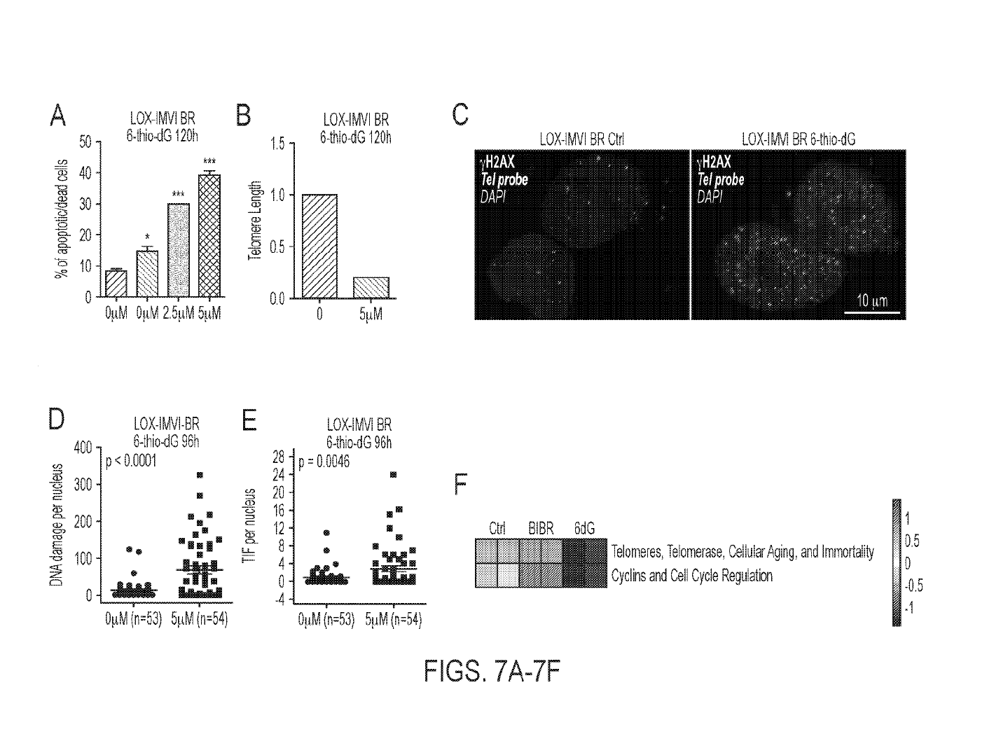

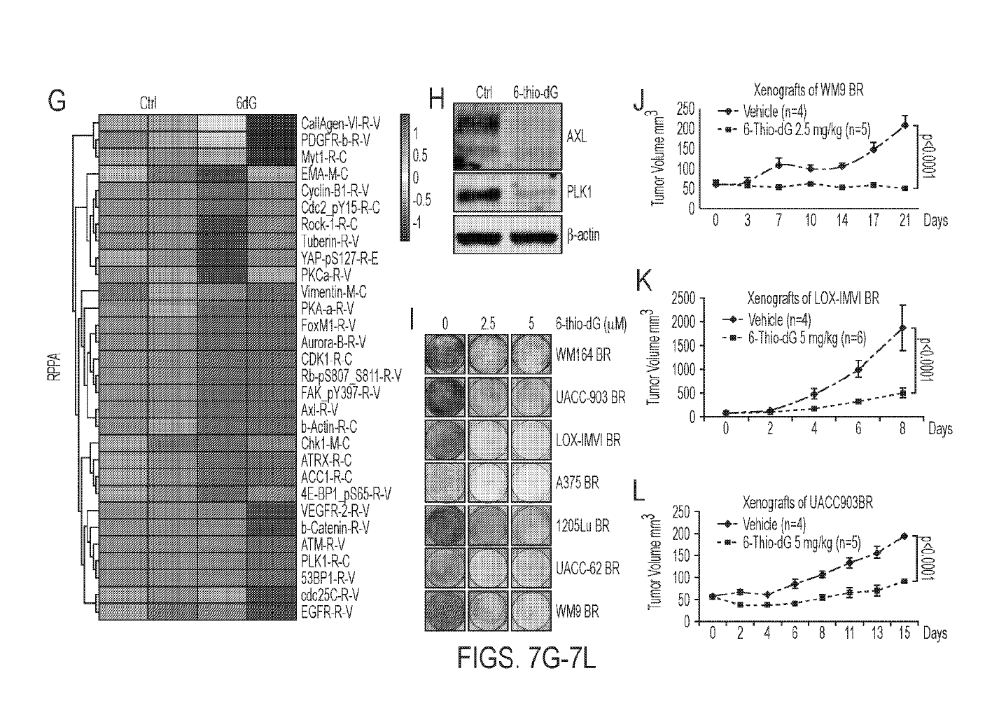

It was observed that 6-thio-dG significantly induced apoptosis and cell death in LOX-IMVI BR cells that acquired resistance to the BRAFi (FIG. 7A). Treatment of LOX-IMVI BR cells with 6-thio-dG for 120 hours led to progressive telomere shortening (FIG. 7B). Compared to the control, 6-thio-dG also induced DNA damage (FIGS. 7C-7D) and led to an increase in telomere dysfunction-induced foci (TIFs) that can be observed as co-localization of a specific γ-H2AX antibody (double strand DNA damage marker) with an in situ telomere specific hybridization probe (FITC-conjugated telomere sequence (TAGGG)₃ peptide nucleic acid) (FIGS. 7C and 7E).

RNA-seq was carried out to profile the transcriptome of LOX-IMVI BR cells treated with 6-thio-dG and found that the ssGSEA demonstrated that “cell cycle” and “telomere signaling” pathways were diminished. This is in line with data obtained from A375 cells treated with 6-thio-dG (FIG. 7F).

Similar to A375 cells treated with 6-thio-dG, the analysis of RPPA data showed that, three major signaling pathways were altered in LOX-IMVI BR cells treated with 6-thio-dG. They were (1) cell cycle: phospho-RB^{Ser807/811}, cyclin B1, CDK1, FOXM1, PLK1, AURKB, Cdc25C, and p-Cdc2^{Y215}; (2) DNA damage response: 53BP1 and CHK1; (3) receptor tyrosine kinase signaling: VEGFR and PDGFRb (FIG. 7G). In addition, other functionally important proteins were also down-regulated, including p-YAP^{S127}, p-FAK^{Y397}, p-4E-BP1^{S65}, and β-catenin (FIG. 7G). To validate the RPPA data, western blotting was used to demonstrate that expression levels of AXL and PLK1 were decreased in LOX-IMVI BR cells treated with 6-thio-dG by (FIG. 7H).

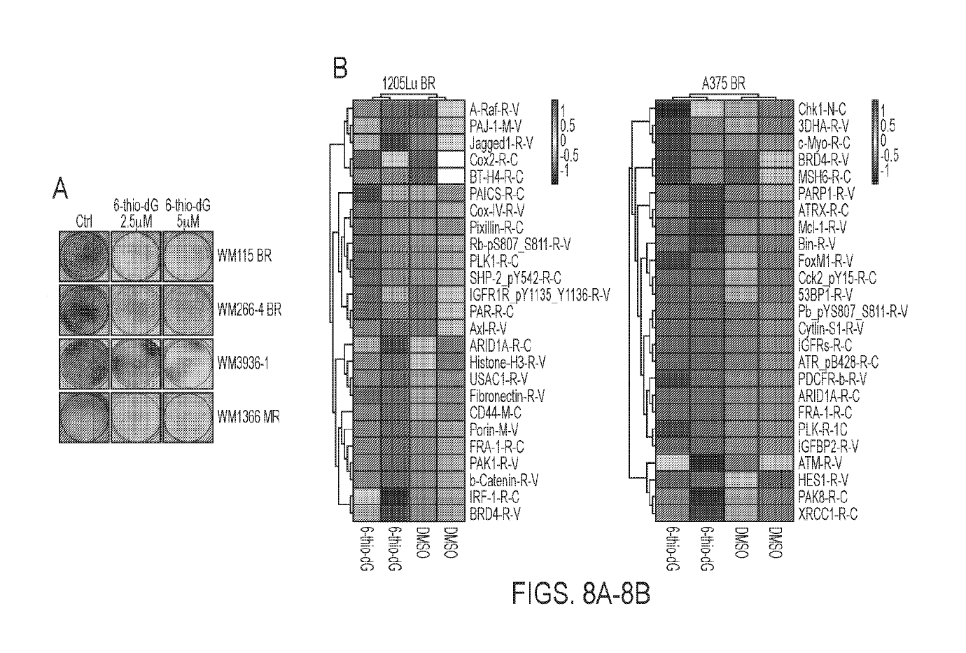

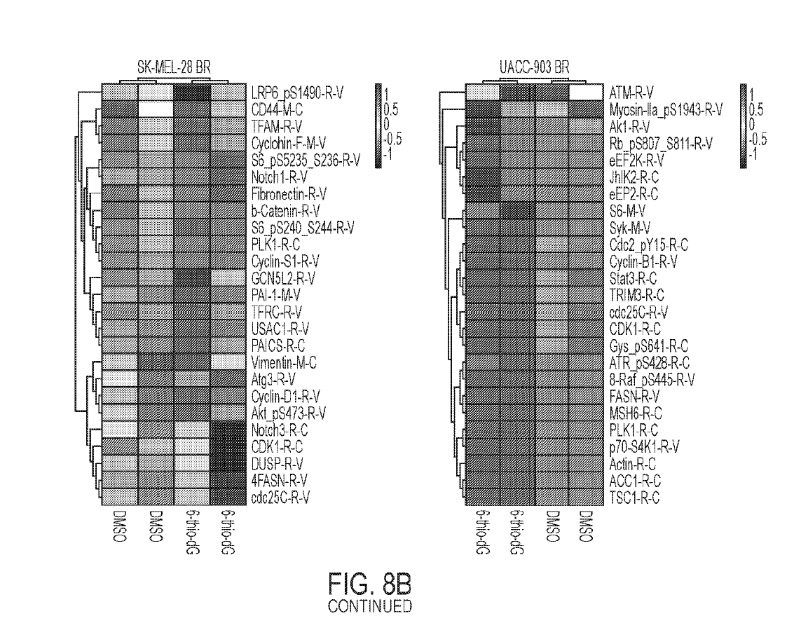

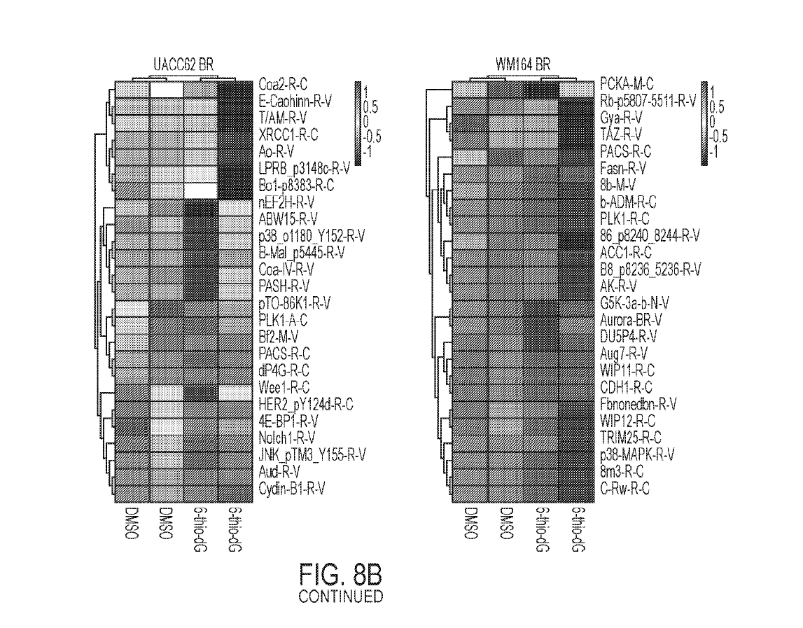

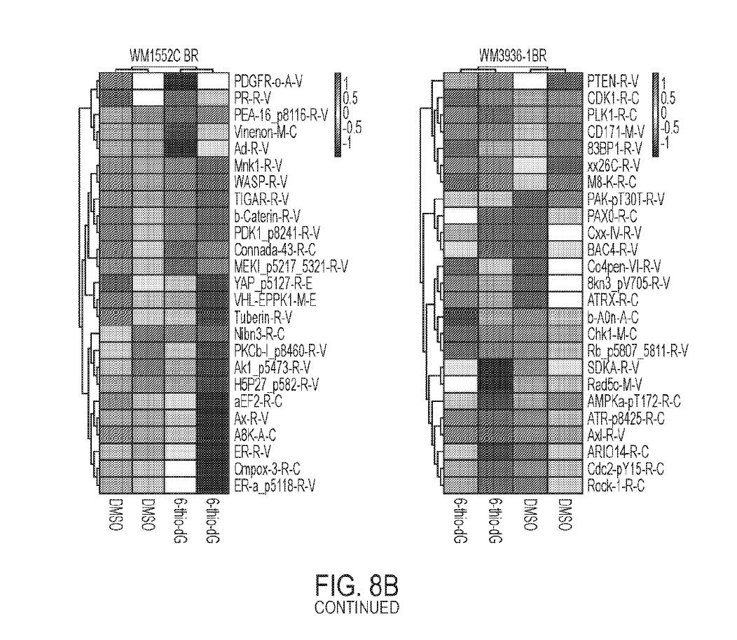

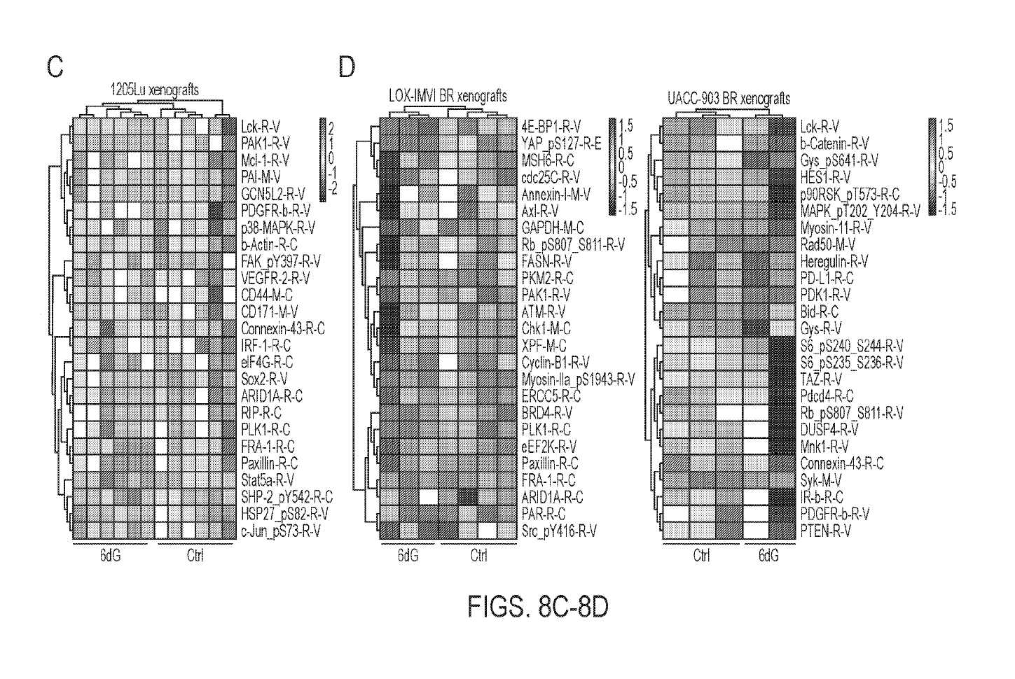

Treatment of ten melanoma cell lines that acquired resistance to PLX4720 and one melanoma cell line (WM1366 MR) that acquired resistance to the MEK inhibitor, MEK162 with 6-thio-dG for 12 days impaired their viabilities (FIGS. 7I and 8A). The RPPA profiling of eight MAPKi-resistant cell lines treated with 6-thio-dG revealed similar observations to those made in LOX-IMVI BR cells (FIG. 8B).

It was found that treatment of A375^{Fucci2}-BR cells that acquired resistance to the BRAF inhibitor or A375^{Fucci2}-CR cells that acquired resistance to the combination of BRAF and MEK inhibitors also led to the arrest of cell cycle progression and the induction of cell death as demonstrated by the time-lapse imaging (data not shown).

Furthermore, it was shown that 6-thio-dG significantly impaired the in vivo growth of three xenografts derived from WM9 BR, LOX-IMVI BR and UACC-903 BR cells (FIGS. 7J-7L). 1205Lu, LOX-IMVI BR and UACC-903 BR xenografts treated with 6-thio-dG in vivo were profiled with RPPA. The analysis of RPPA data confirmed the inhibition of AXL, ARIDIA and PDGFRβ in these xenografts (FIGS. 8C-8D).

The Association of TERT Expression with Overall Survival and Therapy Resistance.

Studies were extended from cell lines to human melanoma by analyzing RNA-seq data of 470 TCGA skin cutaneous melanomas and genome-wide gene expression microarray data of 104 melanomas, 9 nevi, and 7 normal skin samples (GEO accession number GSE46517). Initially, the focus was on the enrichment of two telomere transcriptional gene signatures—“packaging of telomere ends” and “telomere maintenance”. Four additional gene sets were included as benchmarks that are uniquely expressed in melanoma, including “lysosome”, “melanogenesis”, “BRAF targets” and “MEK targets” (Barretina et al., 2012; Cancer Genome Atlas, 2015; Gao et al., 2015; Kabbarah et al., 2010).

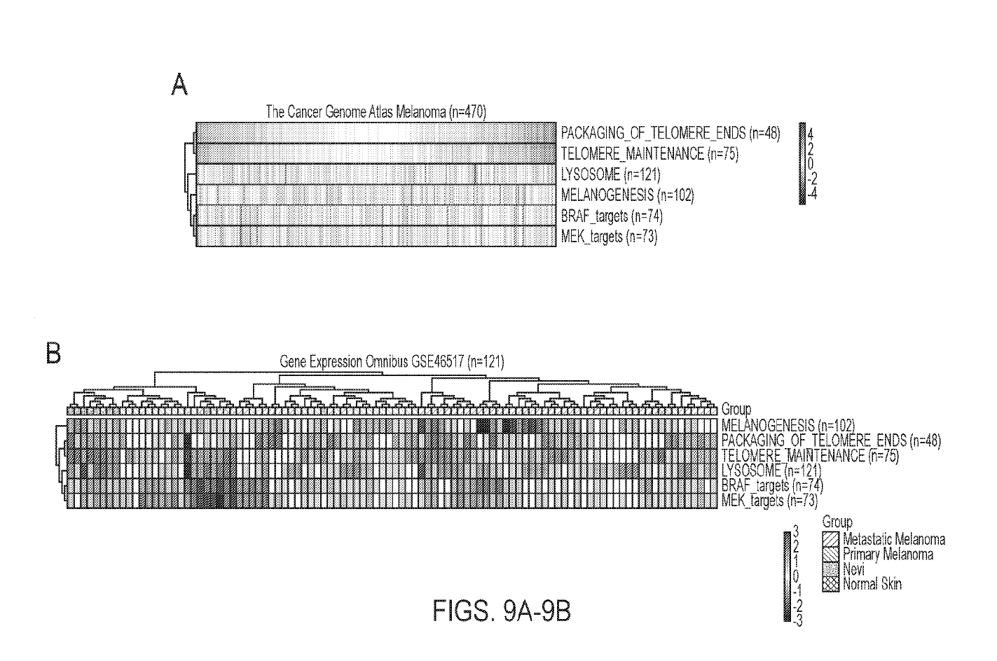

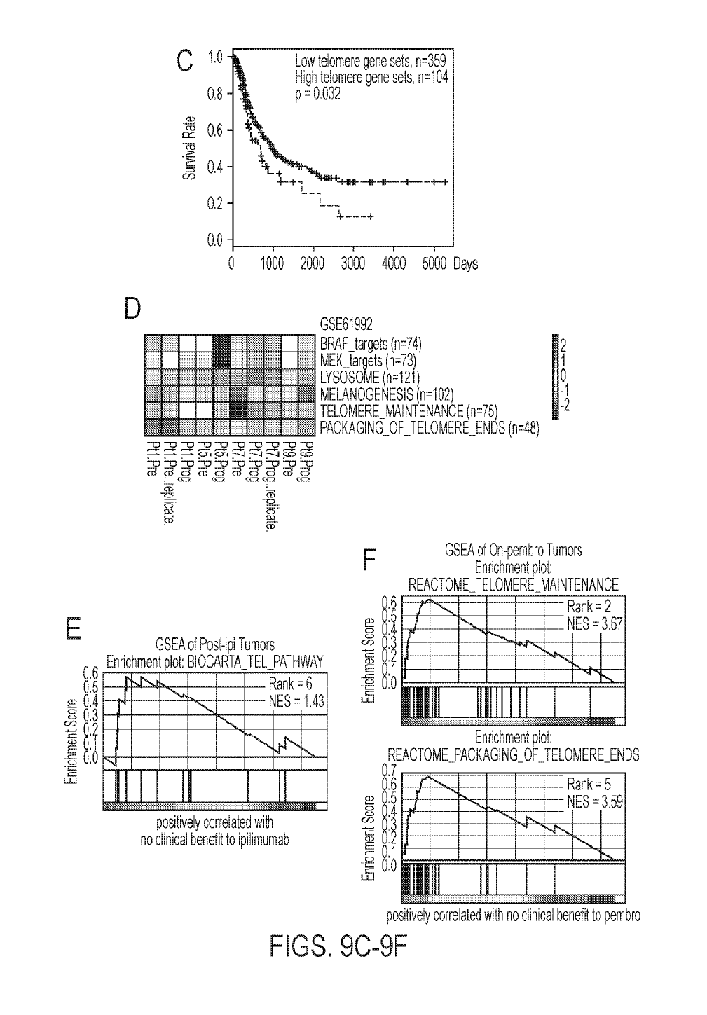

The ssGSEA revealed that “packaging of telomere ends” and “telomere maintenance” were highly enriched in a substantial subset of TCGA melanomas (FIG. 9A). Additionally, the inventors demonstrated that these two telomere transcriptional gene signatures were highly enriched in primary and metastatic melanomas but not in normal skin samples or benign nevi (FIG. 9B). Importantly, the Kaplan-Meier survival analysis revealed that TCGA patients whose melanomas have higher enrichment scores of telomere transcriptional gene signatures are associated with a worse overall survival outcome compared to those whose tumors had lower enrichment scores (FIG. 9C).

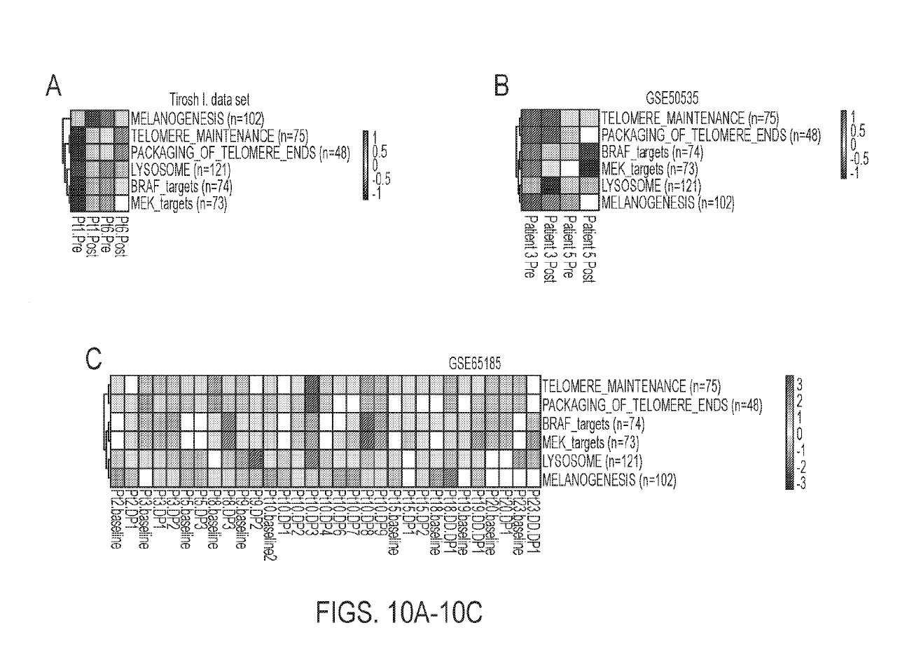

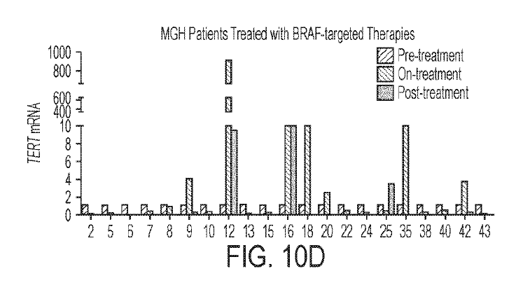

In addition to treatment-naïve human melanoma, paired pre-, on- and post-treatment tumor biopsies derived from patients with metastatic melanoma who were treated with targeted therapies or immune checkpoint blockade therapies were analyzed. The ssGSEA identified that “packaging of telomere ends” and “telomere maintenance” were highly enriched in a subset of post-treatment tumor biopsies procured at the time of disease progression on BRAF inhibitor or the combination therapy of BRAF and MEK inhibitors compared to paired pre-treatment tumor biopsies (FIG. 9D and FIGS. 10A-10C). It was shown that there was a greater than a 2-fold increase in expression of TERT in 7 out of 21 on-treatment tumor biopsies and 3 out of 6 post-treatment tumor biopsies, respectively (FIG. 10D and Table 6).

TABLE 6
Targeted Therapy Patient's Clinical Information

| | | | | | Time To |
|---|---|---|---|---|---|
| | | | | | Progression |
| PT | Mutation | RX | Dose (daily) | Response | (months) |
| 2 | BRAF | vemurafenib | 1920 mg | PR (−60.5%) | 8.5 |
| 4 | BRAF | vemurafenib | 1920 mg | PR (−56%) | 3.5 |
| 5 | BRAF | vemurafenib | 1920 mg | SD (−27%) | 6.5 |
| 6 | BRAF | dabrafenib + | dabrafenib: | PR (−59.9%) | 21 |
| | | trametinib | 300 mg, trametinib: | | |
| | | | 2 mg | | |
| 7 | BRAF | dabrafenib + | dabrafenib: | CR (−100%) | 17, ongoing 47 |
| | | trametinib | 300 mg, trametinib: | | months |
| | | | 2 mg | | |
| 8 | BRAF | dabrafenib + | dabrafenib: | PR (−30%) | 3 |
| | | trametinib | 300 mg, trametinib: | | |
| | | | 1.5 mg | | |
| 9 | BRAF | dabrafenib + | dabrafenib: | PR (−45%) | 7 |
| | | trametinib | 300 mg, trametinib: | | |
| | | | 2 mg | | |
| 10 | BRAF | dabrafenib + | dabrafenib: | SD (−13%) | 3 |
| | | trametinib | 300 mg, trametinib: | | |
| | | | 2 mg | | |
| 11 | BRAF | dabrafenib + | dabrafenib: | PR (−80%) | 10 |
| | | trametinib | 300 mg, trametinib: | | |
| | | | 2 mg | | |
| 12 | BRAF | dabrafenib + | dabrafenib: | PR (−88.9%) | 12, stopped at 20 |
| | | trametinib | 300 mg, trametinib: | | months |
| | | | 2 mg | | |
| 13 | BRAF | dabrafenib + | dabrafenib: | PR (−57.9%) | 9, stroke |
| | | trametinib | 300 mg, trametinib: | | |
| | | | 2 mg | | |
| 15 | BRAF | vemurafenib | 1920 mg | SD (−16.5%) | 6 |
| 16 | BRAF | dabrafenib + | dabrafenib: | SD (−19.5%) | 11 |
| | | trametinib | 300 mg, trametinib: | | |
| | | | 1 mg | | |
| 19 | BRAF | dabrafenib + | dabrafenib: | PR (−48.7%) | ongoing, 36 |
| | | trametinib | 300 mg, trametinib: | | months |
| | | | 2 mg | | |
| 20 | BRAF | vemurafenib | 1920 mg | PR (−51.2%) | 5 |
| 22 | BRAF | dabrafenib + | dabrafenib: | PR (−42%) | 3 |
| | | trametinib | 300 mg, trametinib: | | |
| | | | 2 mg | | |
| 24 | BRAF | vemurafenib | 1920 mg | PR (−53%) | 2 |
| 25 | BRAF | dabrafenib + | dabrafenib: | PR (−64%) | 3 |
| | | trametinib | 150 mg, trametinib: | | |
| | | | 2 mg | | |
| 34 | BRAF | LGX818 + | LGX818: | PR (−48.6%) | stopped drug |
| | | MEK162 | 600 mg, | | after 14 months, |
| | | | MEK162: | | PD at 15 months |
| | | | 45 mg | | |
| 35 | BRAF | LGX818 + | LGX818: | SD (−22.8%) | stopped after 7 |
| | | MEK162 | 600 mg, | | months, PD at 10 |
| | | | MEK162: | | months |
| | | | 45 mg | | |
| 38 | BRAF | vemurafenib | 1920 mg | SD (−24.9%) | 4.3 |
| 40 | BRAF | vemurafenib | 1920 mg | SD | stopped drug |
| | | | | | after 6 months, |
| | | | | | PD at 9 months |
| 42 | BRAF | LGX818 + | LGX818: | PR (−76.1%) | PD at 13 months |
| | | MEK162 | 400 mg, | | |
| | | | MEK162: | | |
| | | | 60 mg | | |
| 43 | BRAF | vemurafenib | 1920 mg | CR (−81.5%) | 13.4 |

To establish a direct link between the activation of telomere signaling axis and resistance to immune checkpoint blockade therapies, RNA-seq data of 14 post-treatment tumor biopsy specimens derived from patients with metastatic melanoma who were treated with ipilimumab were first analyzed (Chiappinelli et al., 2016; Snyder et al., 2014). By comparing tumor biopsy specimens derived from patients experiencing “no clinical benefit” to those derived from patients experiencing “long-term clinical benefit” (Table 7), genes were identified that were differentially expressed between “no clinical benefit” and “long-term clinical benefit” subgroups and then GSEA was carried out in order to identify pathways that were positively associated with the phenotype of “no clinical benefit”. Interestingly, among those highly ranked gene sets is the “BioCarta TEL pathway” that is comprised of genes related to telomeres, telomerase, cellular aging, and immortality (FIG. 9E).

TABLE 7
MSKCC Immunotherapy Patients' Clinical Information

| | | Re- | | | | |
|---|---|---|---|---|---|---|
| PatientID | Histology | sponse | Run | PrePost | Gender | BioSite |
| CR1509 | Melanoma | LB | First | post | F | cutaneous |
| CR7623 | Melanoma | NB | First | post | M | cutaneous |
| CR9699.1 | Melanoma | LB | First | post | M | cutaneous |
| CR9699.2 | Melanoma | LB | First | post | M | cutaneous |
| NR1867 | Melanoma | NB | First | pre | M | acral |
| NR3549 | Melanoma | NB | First | pre | M | cutaneous |
| NR4631 | Melanoma | NB | Second | post | M | cutaneous |
| NR4810 | Melanoma | NB | First | post | F | cutaneous |
| NR4949 | Melanoma | NB | First | post | F | unknown |
| NR5784 | Melanoma | NB | First | pre | F | cutaneous |
| NR8727 | Melanoma | NB | First | pre | M | cutaneous |
| NR9449 | Melanoma | NB | Second | post | F | cutaneous |
| NR9521 | Melanoma | NB | Second | post | M | cutaneous |
| NR9705 | Melanoma | NB | Second | post | F | acral |
| SD0346 | Melanoma | LB | First | post | F | cutaneous |
| SD1494.1 | Melanoma | LB | First | pre | M | cutaneous |
| SD1494.2 | Melanoma | LB | First | pre | M | cutaneous |
| SD2051 | Melanoma | NB | First | pre | F | acral |
| SD2056 | Melanoma | LB | First | pre | F | cutaneous |
| SD5038 | Melanoma | NB | First | post | M | cutaneous |
| SD5118.1 | Melanoma | NB | First | post | F | acral |
| SD5118.2 | Melanoma | NB | First | post | F | acral |
| SD6336.1 | Melanoma | LB | First | post | M | cutaneous |
| SD6336.2 | Melanoma | LB | First | post | M | cutaneous |
| SD6494.1 | Melanoma | NB | First | post | F | unknown |
| SD6494.2 | Melanoma | NB | First | post | F | unknown |
| SD7357.1 | Melanoma | NB | First | post | F | unknown |
| SD7357.2 | Melanoma | NB | First | post | F | unknown |

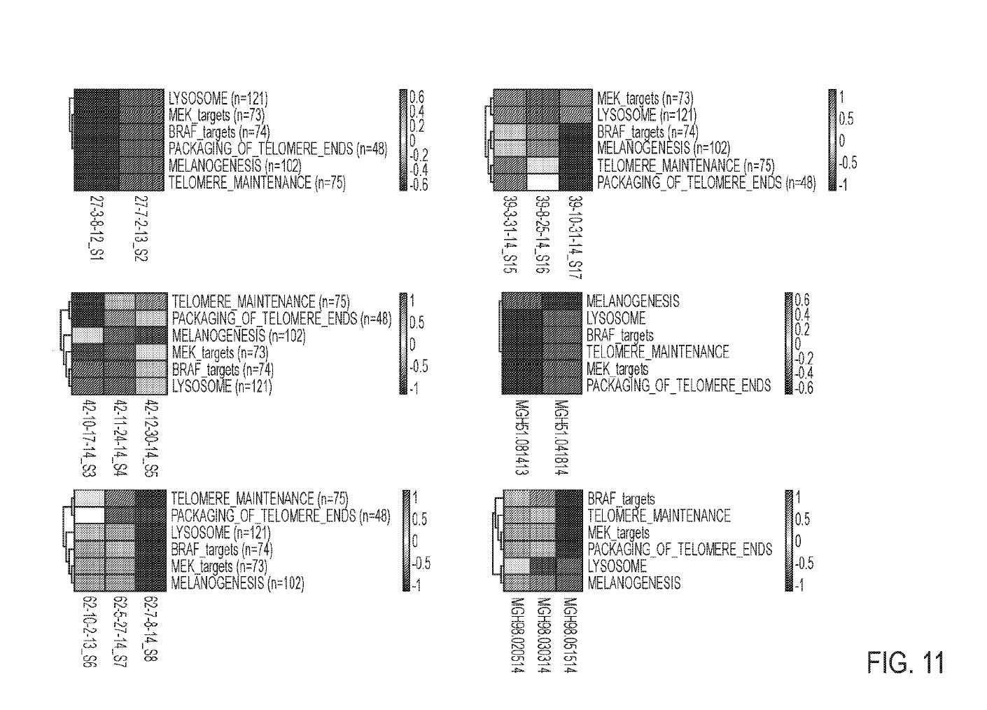

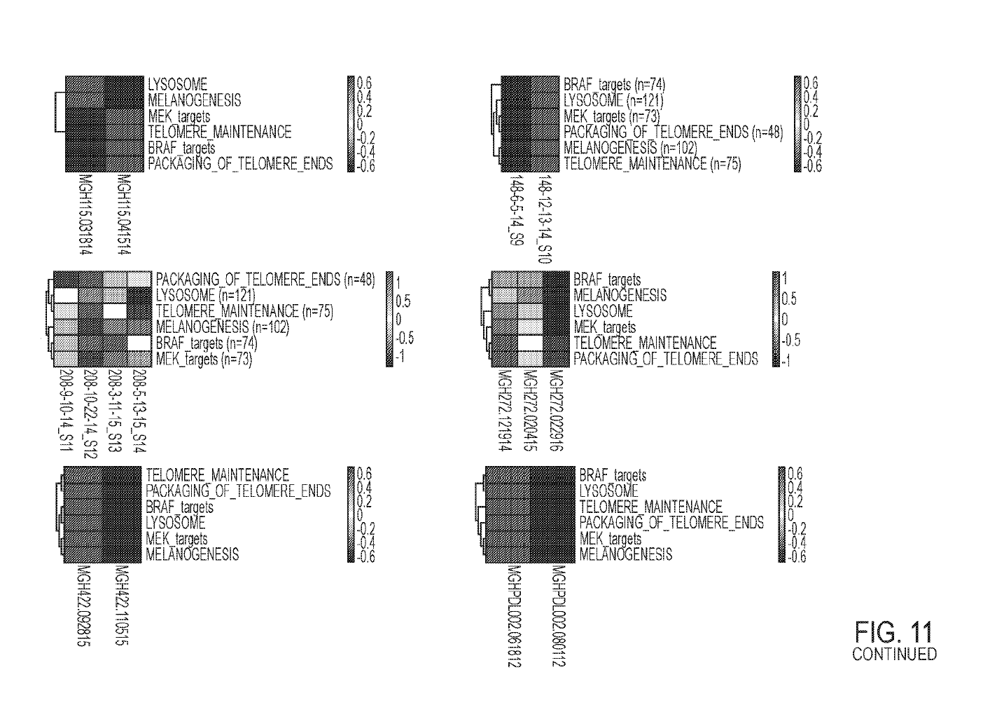

RNA-seq of 31 paired pre-, on- and/or post-treatment tumor biopsies derived from 12 patients who were treated with various immune checkpoint blockade therapies were carried out. The best response for most of patients was progressive disease. The ssGSEA identified that “packaging of telomere ends” and “telomere maintenance” were highly enriched in 7 out 12 patients' on- or post-treatment tumor biopsies (FIG. 11). For instance, patient #39 was treated with pembrolizumab with the best response being progressive disease. Prior to pembrolizumab, this patient progressed on vemurafenib, IL-2, ipilimumab, and dabrafenib plus trametinib. It was observed that “telomere maintenance” and “packaging of telomere ends” were highly enriched in this patient's on-treatment tumor biopsy compared to the paired pre-treatment tumor biopsy (FIG. 9F and Table 8).

TABLE 8
MGH Immunotherapy Patients' Clinical Information

| MGH | | | | | | | | |
|---|---|---|---|---|---|---|---|---|
| Patient | | | Muta- | | | | | |
| ID | Date | SampleID | tion | RX-1 | RX-2 | Response | start_date | notes |
| 27 | Mar. 8, 2012 | 27_030812 | BRAF | BRAFi−>PD1 | post-BRAFi | PD | Jun. 1, 2013 | |
| | | | mut | | (479 days); | | | |
| | | | | | pre-PD1 | | | |
| 27 | Jul. 2, 2013 | 27_070213 | | | post-BRAFi | | | |
| | | | | | (960 days); | | | |
| | | | | | post-PD1 | | | |
| | | | | | (31 days) | | | |
| 39 | Mar. 31, 2014 | 39_033114 | BRAF | Vem−>IL2−>IPI−>dab + | post-BRAFi | PD | Vem: | |
| | | | mut | tra−>PD1 | (393 days); | (Mixed | Mar. 3, 2013; | |
| | | | | | post-IL2 (388 | response: | IL2: | |
| | | | | | days); post-IPI | PR of | Mar. 18, 2013; | |
| | | | | | (153 days); | bulky | IPI: | |
| | | | | | pre-dab + tra; | nodes but | Oct. 29, 2013- | |
| | | | | | pre-PD1 | PD soft | Dec. 30, 2013; | |
| | | | | | | tissue) | dab + tra: | |
| | | | | | | | Jul. 2, 2014; PD1: | |
| | | | | | | | Aug. 4, 2014 | |
| 39 | Aug. 25, 2014 | 39_082514 | | | post-BRAFi | X5 | | |
| | | | | | (540 days); | | | |
| | | | | | post-IL2 (535 | | | |
| | | | | | days); post-IPI | | | |
| | | | | | (300 days); | | | |
| | | | | | post-dab + tra | | | |
| | | | | | (54 days); | | | |
| | | | | | post-PD1 | | | |
| | | | | | (21 days) | | | |
| 39 | Oct. 31, 2014 | 39_103114 | | | post-BRAFi | | | |
| | | | | | (607 days); | | | |
| | | | | | post-IL2 (602 | | | |
| | | | | | days); post-IPI | | | |
| | | | | | (367 days); | | | |
| | | | | | post-dab + tra | | | |
| | | | | | (121 days); | | | |
| | | | | | post-PD1 | | | |
| | | | | | (88 days) | | | |
| 42 | Oct. 17, 2014 | 42_101714 | BRAF | IPI−>dab + | post-IPI (176 | X2 | IPI: | |
| | | | mut | tra−>PD1 | days); post- | | Apr. 24, 2014, | |
| | | | | | dab + tra (64 | | 4th dose | |
| | | | | | days); pre- | | Jun. 27, 2014; | |
| | | | | | PD1 | | dab + tra: | |
| | | | | | | | Aug. 14, 2014- | |
| | | | | | | | October 2014; | |
| | | | | | | | PD1: | |
| | | | | | | | Oct. 17, 2014- | |
| | | | | | | | Jan. 9, 2015 PD | |
| 42 | Nov. 24, 2014 | 42_112414 | | | post-IPI (214 | PD | | |
| | | | | | days); post- | | | |
| | | | | | dab + tra (102 | | | |
| | | | | | days); post- | | | |
| | | | | | PD1 (38 days) | | | |
| 42 | Dec. 30, 2014 | 42_123014 | | | post-IPI (250 | | | |
| | | | | | days); post- | | | |
| | | | | | dab + tra (138 | | | |
| | | | | | days); post- | | | |
| | | | | | PD1 (74 days) | | | |
| | | | | | [PD after 84 | | | |
| | | | | | days] | | | |
| 51 | Aug. 14, 2013 | 51_081413 | | | pre-PDL1 | | | |
| 51 | Apr. 18, 2014 | 51_041814 | | PD-L1 | post-PDL1 | SD | | |
| | | | | | (224 days) | (−4.8%)/ | | |
| | | | | | | slow PD | | |
| 62 | Oct. 2, 2013 | 62_100213 | GNAQ | IPI−>PD1−>CDK4/6i + | pre-IPI; pre- | slow, | IPI: | |
| | | | mut | MEKi | PD1; pre- | steady | Oct. 4, 2013- | |
| | | | | | CDK4/6i + | PD | Dec. 4, 2013 PD; | |
| | | | | | MEKi | | PD1: | |
| | | | | | | | May 24, 2014- | |
| | | | | | | | Jul. 8, 2014; | |
| | | | | | | | CDK4/6 + MEKi: | |
| | | | | | | | Jan. 7, 2015 - SD | |
| 62 | May 27, 2014 | 62_052714 | | | post-IPI (235 | | | |
| | | | | | days); post- | | | |
| | | | | | PD1 (3 days); | | | |
| | | | | | pre- | | | |
| | | | | | CDK4/6i + | | | |
| | | | | | MEKi | | | |
| 62 | Jul. 8, 2014 | 62_070814 | | | post-IPI (277 | | | |
| | | | | | days); post- | | | |
| | | | | | PD1 (45 | | | |
| | | | | | days); pre- | | | |
| | | | | | CDK4/6i + | | | |
| | | | | | MEKi | | | |
| 98 | Feb. 5, 2014 | 98_020514 | NA | PD1−>IPI | post-PD1 (83 | PR | NIVO: | |
| | | | | | days); pre-IPI | (−62.1%), | Nov. 14, 2013- | |
| | | | | | | mixed | Jan. 23, 2014; | |
| | | | | | | response | IPI: | |
| | | | | | | | Feb. 6, 2014- | |
| | | | | | | | Apr. 10, 2014; | |
| | | | | | | | maintenance | |
| | | | | | | | NIVO: | |
| | | | | | | | May 2014- | |
| 98 | Mar. 3, 2014 | 98_030314 | | | post-PD1 (109 | | | |
| | | | | | days); post-IPI | | | |
| | | | | | (25 days) | | | |
| 98 | May 15, 2014 | 98_051514 | | | post-PD1 (182 | | | |
| | | | | | days); post-IPI | | | |
| | | | | | (98 days) | | | |
| 98 | Jun. 11, 2015 | 98_061115 | | | post-PD1 (574 | | | |
| | | | | | days); post- | | | |
| | | | | | IPI (490 days) | | | |
| 115 | Mar. 18, 2014 | 115_031814 | | IPI−>PD1−>PD1 + | pre-IPI; pre- | PR | IPI | |
| | | | | KIR | PD1; pre-KIR | (−37.7%)/ | (Mar. 24, 2014, | |
| | | | | | | PD CNS | 3 cycles, PD1: | |
| | | | | | | | May 25, 2014 | |
| | | | | | | | PD), PD1 + KIR | |
| | | | | | | | (Aug. 25, 2014-, | |
| | | | | | | | PD CNS, PR | |
| | | | | | | | elsewhere, | |
| | | | | | | | remaining on | |
| | | | | | | | trial) | |
| 115 | Apr. 15, 2014 | 115_041514 | | | post-IPI (22 | | | |
| | | | | | days); pre- | | | |
| | | | | | PD1; pre-KIR | | | |
| 115 | Jun. 5, 2015 | 115_060515 | | | post-IPI (438 | | | |
| | | | | | days); post- | | | |
| | | | | | PD1 (376 | | | |
| | | | | | days); post- | | | |
| | | | | | KIR (284 days) | | | |
| 148 | Jun. 5, 2014 | 148_060514 | NA | PD1 + KIR | pre-PD1; pre- | PD | PD1/KIR: | |
| | | | | | KIR | | Jun. 5, 2014- | |
| | | | | | | | Nov. 14, 2014 PD | |
| 148 | Dec. 31, 2014 | 148_123114 | | | post-PD1 (209 | | | |
| | | | | | days); post- | | | |
| | | | | | KIR (209 days) | | | |
| 208 | Sep. 10, 2014 | 208_091014 | NA | IPI−>PD1 | pre-IPI; pre- | PD, | prior limb | |
| | | | | | PD1 | mixed | perfusion; IPI: | |
| | | | | | | response | Sep. 10, 2014 | |
| | | | | | | | C1d1- | |
| | | | | | | | Nov. 12, 2014 | |
| | | | | | | | 4 doses, mixed | |
| | | | | | | | response, PD | |
| | | | | | | | March 2015; | |
| | | | | | | | PD1: | |
| | | | | | | | Mar. 11, 2015- | |
| 208 | Oct. 22, 2014 | 208_102214 | | | post-IPI (42 | | | |
| | | | | | days); pre- | | | |
| | | | | | PD1 | | | |
| 208 | Mar. 11, 2015 | 208_031115-1 | | | post-IPI (182 | X2 | | |
| | | | | | days); pre- | | | |
| | | | | | PD1 | | | |
| 208 | Mar. 11, 2015 | 208_031115-2 | | | post-IPI (182 | X2 | | |
| | | | | | days); pre- | | | |
| | | | | | PD1 | | | |
| 208 | May 13, 2015 | 208_051315 | | | post-IPI (245 | | | |
| | | | | | days); post- | | | |
| | | | | | PD1 (63 days) | | | |
| 272 | Dec. 19, 2014 | 272_121914 | NA | IPI−>PD1 | pre-IPI | PR | IPI: | Patient was slow |
| | | | | | | (eventu- | Dec. 19, 2014- | progressor (had |
| | | | | | | ally) | Feb. 25, 2015 | initial growth), |
| | | | | | | | (4 cycles); PD1: | but after Tx |
| | | | | | | | Mar. 20, 2015 | tumor went |
| | | | | | | | | away (responder) |
| 272 | Feb. 4, 2015 | 272_020415 | | | post-IPI (47 | | | |
| | | | | | days); pre- | | | |
| | | | | | PD1 | | | |
| 422 | Sep. 28, 2015 | 422_092815 | | | pre-IPI; pre- | | | |
| | | | | | PD1 | | | |
| 422 | Nov. 5, 2015 | 422_110515 | | | post-IPI; post- | | | |
| | | | | | PD1 | | | |
| PDL002 | Jun. 18, 2012 | PDL002_061812 | BRAF | PDL1 | pre-PDL1 | SD(−0.7%) | Jun. 18, 2012 | |
| | | | mut | | | | | |
| PDL002 | Aug. 1, 2012 | PDL002_080112 | | | post-PDL1 | | | |
| | | | | | (44 days) | | | |
| PDL002 | Feb. 12, 2013 | PDL002_021213 | | | | | | |

6-Thio-dG Significantly Impairs the Cell Viability, Proliferation and Tumor Growth of Melanoma Cells that are Resistant to Immune Checkpoint Inhibitors.

Having demonstrated the efficacy of 6-thio-dG in inhibiting the in vivo growth of MAPKi-resistant tumors, the ability of 6-thio-dG to inhibit the in vivo growth of melanoma cells that are resistant to immune checkpoint inhibitors was investigated. Two short-term primary cultures, T708-13-456-3-3 and T708-13-456-5-3, were established, respectively, from two BRAF^{V600E} melanoma breast metastases that were surgically removed from a patient who progressed on sequential therapies including radiation therapy, ipilimumab, temozolomide, and pembrolizumab. Next, a short-term primary culture, 15-1761-1-2 derived from a metastatic melanoma in a left axillary lymph node, was established. These cells had a NRAS^{61R} mutation that was derived from a patient who first progressed on pembrolizumab and subsequently on the combination of ipilimumab plus nivolumab. Finally, PDXs from two NRAS^{Q61R} brain metastases derived from a patient with metastatic melanoma were established, and two PDXs-derived cell lines, WM4265-1 and WM4265-2, were subsequently established. This patient progressed on sequential therapies, including cisplatin, vinblastine, temozolomide, interleukin-2, IFN α-2b, ipilimumab, and pembrolizumab.

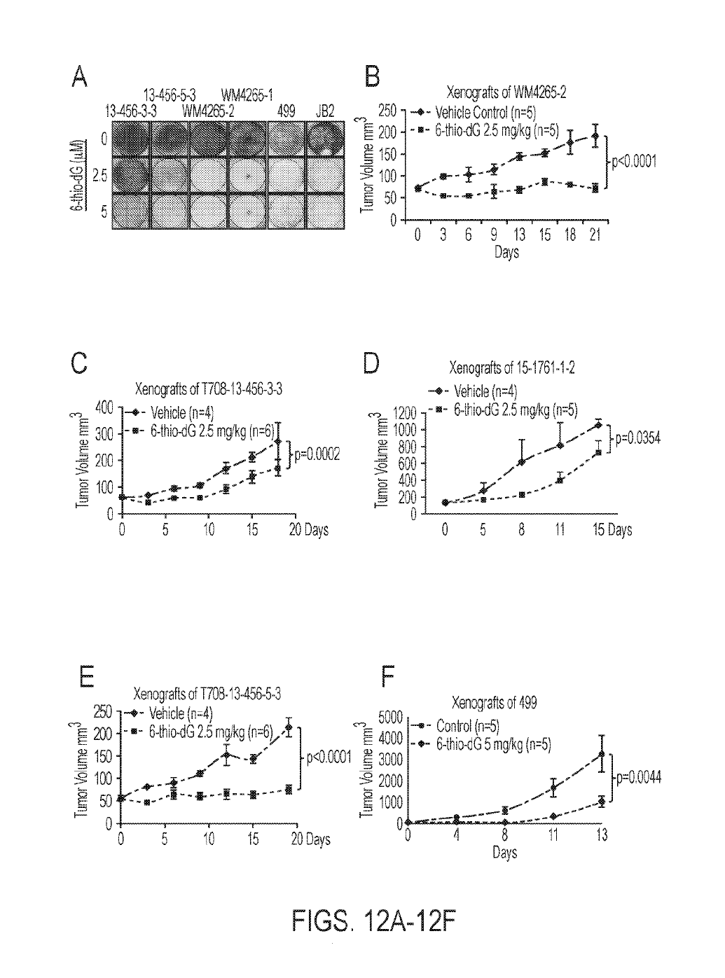

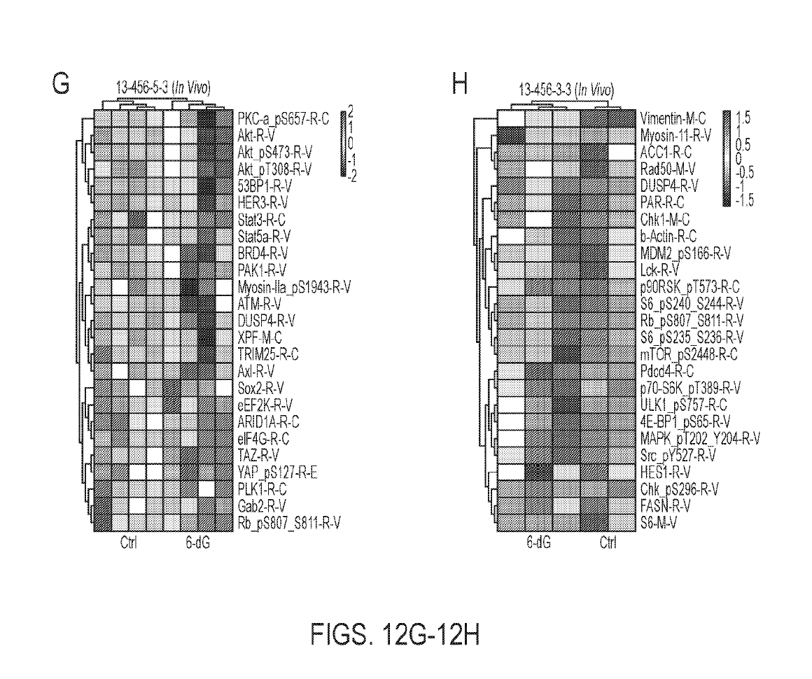

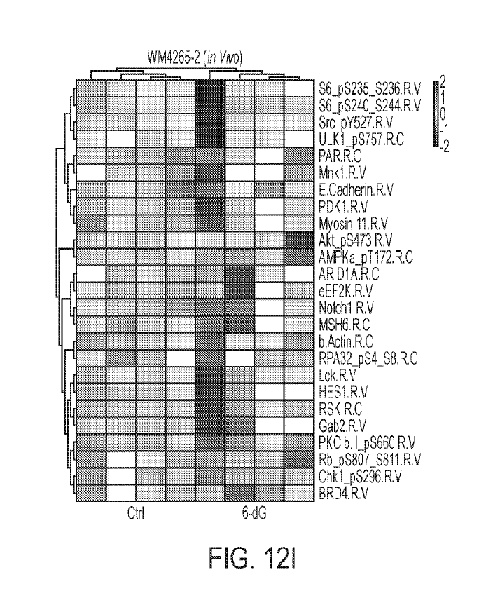

In addition to therapy-resistant human melanoma, there was access to two derivatives of the mouse melanoma cell line B16. The first was 499 cell line that was resistant to radiation therapy and anti-CTLA-4; and the second cell line was JB2 that differs from 499 in which PD-L was knocked out (Benci et al., 2016). Those therapy-resistant cells including 13-456-3-3, 13-456-5-3, WM4265-1, WM4265-2, 499 and JB2 were treated with 6-thio-dG and it was observed that the prolonged treatment with 6-thio-dG for 12 days markedly inhibited in vitro cell proliferation (FIG. 12A).

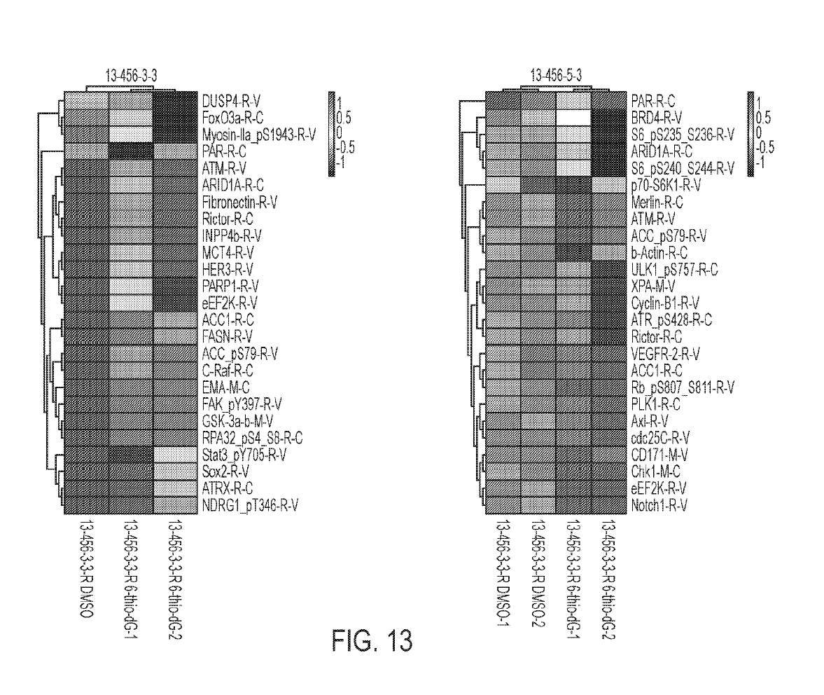

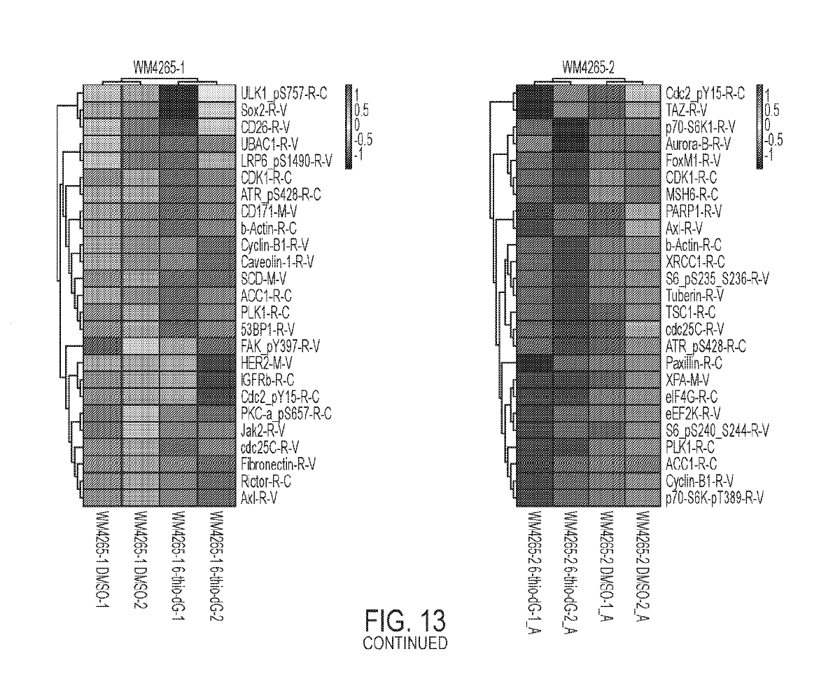

Furthermore, anti-tumor activity of 6-thio-dG in five xenografts, including WM4265-2, 13-456-3-3, 15-1761-1-2, 13-456-5-3 and 499 was shown (FIGS. 12B-12F). Three representative xenografts were profiled that had been treated with 6-thio-dG using RPPA and showed that therapy-resistant markers, AXL, ARIDIA and PLK1 were down-regulated in 13-456-5-3 xenografts (FIG. 12G); mTOR-S6 signaling axis was down-regulated in 13-456-3-3 xenografts (FIG. 12H); and the therapy-resistant marker, ARIDIA and S6 signaling axis were down-regulated in WM4265-2 xenografts (FIG. 12I). Similar observations were also made in 13-456-3-3, 13-456-5-3, WM4265-1 and WM4265-2 cells that were treated with 6-thio-dG in vitro (FIG. 13). In summary, these results demonstrate that 6-thio-dG significantly impaired the in vivo growth of xenografts derived from various therapy-resistant melanoma models.

6-Thio-dG Significantly Impairs Cell Viability, Proliferation and Tumor Growth of Therapy-Resistant Mouse Pancreatic Cancer Cells and Human Ovarian Cancer Cells.

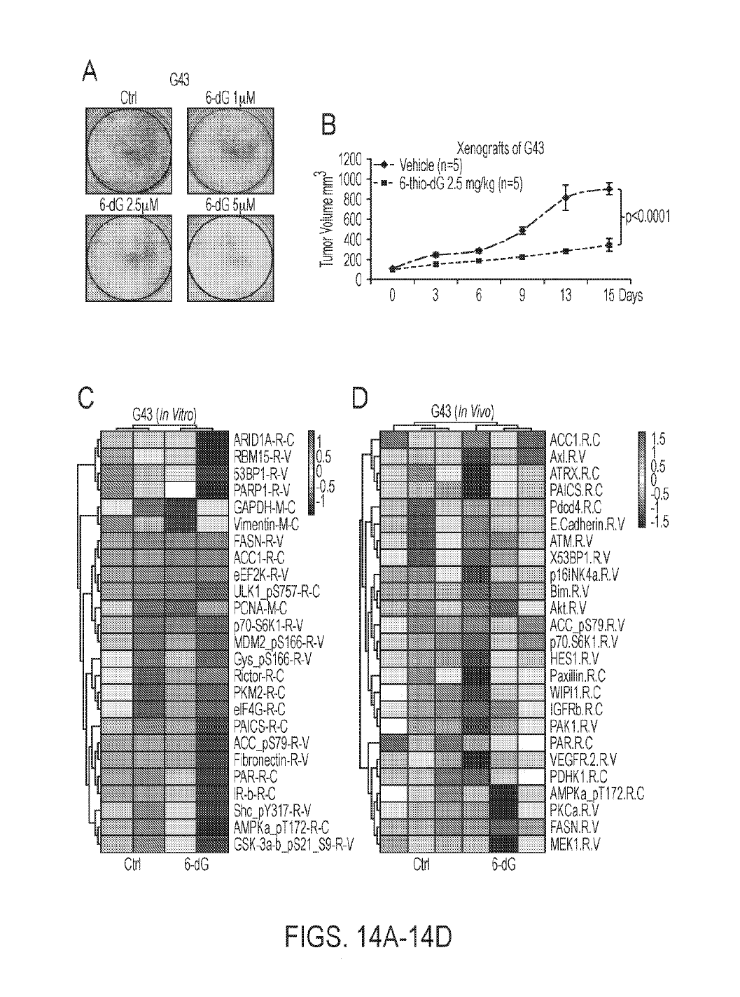

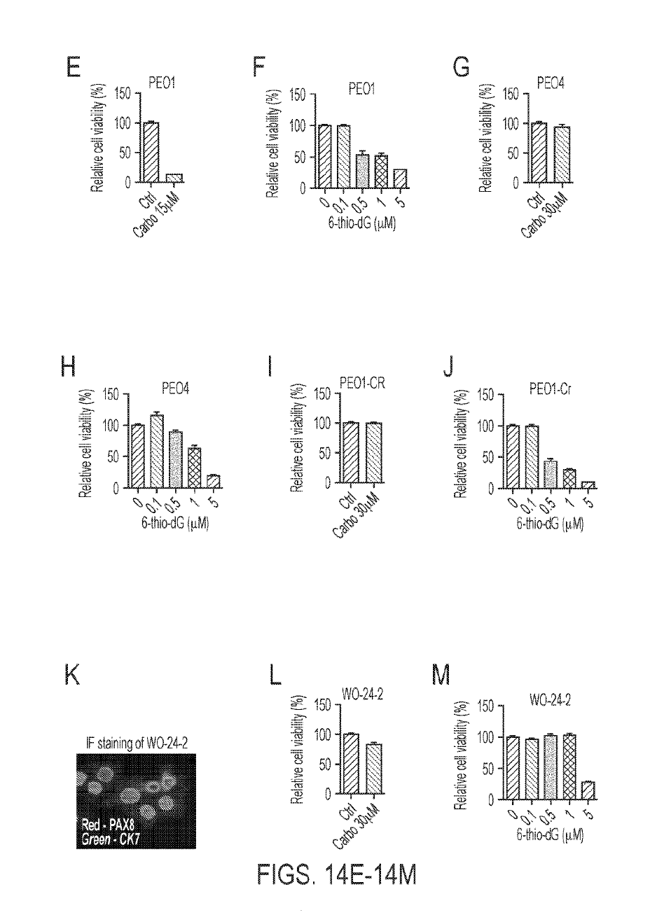

This work was further extended to non-melanoma therapy-resistant cancer cells in order to evaluate the efficacy of 6-thio-dG more broadly. The mouse pancreatic cancer cell line G43 was established from mouse pancreatic tumors harboring mutations in KRAS and P53 that progressed on radiation therapy and subsequently ipilimumab. The in vitro treatment of G43 cells with 6-thio-dG for 9 days impaired cell viability (FIG. 14A). It was tested if 6-thio-dG had in vivo anti-tumor activity and therefore xenografts of G43 tumors were treated with 6-thio-dG for 15 days. It was demonstrated that 6-thio-dG significantly delayed the growth of G43 tumors (p<0.0001) (FIG. 14B). To identify which proteins were down-regulated in G43 cells treated with 6-thio-dG, RPPA profiling was conducted and demonstrated that ARIDIA was down-regulated in G43 cells treated with 6-thio-dG in vitro (FIG. 14C) which was also found in therapy-resistant melanoma cells treated with 6-thio-dG. The analysis of RPPA data revealed that AXL, IGF-IRβ and VEGFR were down-regulated in G43 tumors treated with 6-thio-dG in vivo (FIG. 14D) which was also found in therapy-resistant melanoma cells treated with 6-thio-dG.

Additionally, the efficacy of 6-thio-dG was tested using two human ovarian cancer cell lines, PEO4 and PEO1-CR. PEO4 is a derivative line of BRCA1/2-mutant PEO1 that regains homologous recombination by BRCA reversion mutation. PEO1 was sensitive to carboplatin, whereas PEO4 was intrinsically resistant to carboplatin and PEO1-CR acquired resistance to carboplatin (FIGS. 14E-14G). Importantly, all three cell lines were sensitive to 6-thio-dG after the 5-day treatment (FIGS. 14H-14J).

A short-term primary culture, WO-24-2 from a therapy-resistant human ovarian cancer, was also established. The WO-24-2 tumor was resected from a patient with high grade serous ovarian cancer who underwent an interval debulking surgery after receiving neoadjuvant chemotherapy with platinum/taxane and subsequently progressed on platinum chemotherapy. Immunofluorescence staining of PAX8 and cytokeratin 7 confirmed that these were indeed human ovarian cancer cells (FIG. 14K) (George et al., 2017). It was demonstrated that the WO-24-2 short-term primary culture was resistant to carboplatin (FIG. 14L). Finally, WO-24-2 cells were treated with 6-thio-dG at 5 μM and it was found that cell viability was substantially inhibited (FIG. 14M).

Taken together, this data highlights the impressive anti-tumor activity of 6-thio-dG in non-melanoma therapy-resistant pancreatic and ovarian cancer cells, suggesting that 6-thio-dG may be used as a viable therapeutic approach in additional preclinical models of therapy-resistant cancers.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference:
Barretina et al. Nature 483, 603-607, 2012.
Benci et al. Cell 167, 1540-1554 e1512, 2016.
Cancer Genome Atlas, N. Cell 161, 1681-1696, 2015.
Chiappinelli et al. Cell 164, 1073, 2016.
Gao et al. Nat Med 21, 1318-1325, 2015.
George et al. JCI Insight 2, e89760, 2017.
Kabbarah et al. PLoS One 5, e10770, 2010.
Kim et al. Targeting the ATR/CHK1 Axis with PARP Inhibition Results in Tumor Regression in BRCA-Mutant Ovarian Cancer Models. Clin Cancer Res. 2016.
Mender et al. Cancer Discov 5, 82-95, 2015.
Muller et al. Nat Commun 5, 5712, 2014.
Nazarian et al. Nature 468, 973-977, 2010.
Snyder et al. N Engl J Med 371, 2189-2199, 2014.
Tirosh et al. Science 352, 189-196, 2016.

1. A method of treating a subject with cancer comprising administering to said subject a therapeutically effective amount of 6-thio-2′-deoxyguanosine (6-thio-dG), wherein cells of said cancer are telomerase-positive and exhibit (a) one or more TERT promoter mutations, and/or (b) enriched telomere transcriptional signature(s).

2. A method of treating a subject with melanoma comprising administering to said subject a therapeutically effective amount of 6-thio-2′-deoxyguanosine (6-thio-dG), wherein melanoma is resistant to an immunotherapy and/or MAPKi therapy.

3. The method of claim 1, wherein said subject has had disease progression during or after platinum-based therapy, radiotherapy, immunotherapy and/or MAPKi therapy.

4. The method of claim 2, wherein the immunotherapy is an immune checkpoint inhibitor, such as anti-CTLA4 therapy or anti-PD1 therapy.

5. The method of claim 2, wherein the MAPKi therapy is an anti-MEK therapy, an anti-Raf therapy, and anti-p38 MAPK therapy, and anti-JNK therapy, and anti-ERK therapy, or an anti MNK therapy, such as vemurafenib, sorafenib, dabrafenib, trametinib, selumetinib, losapimod, GSK2118436, PD0325901, PLX4032 or PLX4720.

6. The method of claim 1, wherein the cancer is a B-Raf-mutated cancer.

7. (canceled)

8. The method of claim 1, wherein the cancer is a lung cancer, a melanoma, pancreatic cancer or an ovarian cancer.

9. The method of claim 1, wherein said enriched telomere transcription signature is a telomere maintenance signature or a packaging of telomere ends signature.

10. The method of claim 1, wherein a therapeutically effective amount of 6-thio-dG is between about 0.5 mg/kg and 5.0 mg/kg.

11. The method of claim 1, wherein 6-thio-dG is administered more than once.

12. (canceled)

13. The method of claim 1, wherein 6-thio-dG is administered systemically, such as orally or intravenously.

14. The method of claim 1, wherein 6-thio-dG is administered intratumorally, or local or regional to a tumor site.

15. The method of claim 1, wherein the subject is a human subject.

16. The method of claim 1, wherein the subject is a non-human mammalian subject.

17. The method of claim 1, further comprising treating said subject with a second cancer therapy.

18-20. (canceled)

21. The method of claim 1, wherein treatment results in one or more of impaired cancer cell viability, cancer cell apoptosis, cancer cell senescence in surviving cancer cells, and progressively shortened telomeres in surviving cancer cells.

22. The method of claim 1, wherein treatment results in one or more of increased subject survival, reduced tumor burden, reduction in primary tumor size, reduced metastasis, induction of remission, reduced subject hospitalization and increased subject comfort.

23. The method of claim 1, further comprising assessing a cancer cell from said subject for one or more of (a) TERT promoter mutation, (b) enriched telomere transcriptional signature(s), (c) increased AXL expression, (d) increased PDGFRβ expression, and/or (e) one or more B-Raf mutations.

24. The method of claim 23, wherein said enriched telomere transcription signature is a telomere maintenance signature or a packaging of telomere ends signature.

25. The method of claim 1, wherein said cancer is recurrent, metastatic and/or multi-drug resistant.
